(19) United States
(12) Patent Application Publication
(10) Pub. No.: US 2021/0341484 A1
(43) Pub. Date: Nov. 4, 2021

(54) Methods for Detecting Cancer-Related Cell Populations, Screening for Metastatic Cancer and Treatments Thereof

(71) Applicant: IncellDx, Inc., San Carlos, CA (US)

(72) Inventors: Bruce K. Patterson, San Carlos, CA (US); Amanda Noel Chargin, San Carlos, CA (US); Xiaoyang Wang, San Carlos, CA (US); Pin-I Chen, San Carlos, CA (US)

(21) Appl. No.: 17/255,118

(22) PCT Filed: Jul. 1, 2019
(86) PCT No.: PCT/US2019/040120

§ 371 Date: Dec. 22, 2020

Related U.S. Application Data
(60) Provisional application No. 62/693,235, filed on Jul. 2, 2018.
(60) Provisional application No. 62/792,045, filed on Jan. 14, 2019.

(51) Int. Cl. G01N 33/574; G01N 15/14

(52) U.S. Cl. CPC G01N 33/57484; A61K 45/06; G01N 2800/7028; G01N 15/14

(57) ABSTRACT

Provided are methods involving the assaying of a labeled cell suspension, e.g., to detect cancer-related cells, populations ON thereof and/or screen for metastatic cancer. Labeled cell suspensions may be assayed to detect whether a tumor infiltrating lymphocyte (TIL) population is present in a cellular suspension of a subject. Cancer-related cell populations of interest include, e.g., those expressing one or more markers, including where the markers are members of a marker panel. Markers assayed may vary depending on the context and may include protein markers, nucleic acid markers, cell cycle markers, DNA content markers, and the like. Useful markers include one or more immune checkpoint markers and/or one or more immune cell-type markers, including where the marker(s) assayed are part of one or more panels of markers. Also provided are methods of treating a subject for a neoplasia based on the outcome of an assay of a labeled cell suspension of sample from a subject. Kits for practicing the described methods are also provided.

### CROSS-REFERENCE TO RELATED APPLICATIONS

Pursuant to 35 U.S.C. § 119 (e), this application claims priority to the filing dates of U.S. Provisional Patent Application Ser. No. 62/693,235 filed Jul. 2, 2018 and 62/792,045 filed Jan. 14, 2019, the disclosures of which applications are herein incorporated by reference.

### INTRODUCTION

Cancer remains one of the leading causes of death globally, with an estimated 12.7 million annual cases around the world affecting both sexes equally. This number is expected to increase to 21 million by 2030. As an example, lung cancer is the number one cause of cancer deaths in both men and women in the U.S. and worldwide. Non-small cell lung cancers (NSCLC) are the most common type, accounting for about 80-85% of lung cancers. Most NSCLC cases have a poor prognosis, since most patients are normally at an advanced stage of cancer development when diagnosed.

Traditionally, lung cancers have been treated with surgical removal, radiation therapy, and chemotherapy. Advances in these treatments have been very slow and have provided limited improvement in the 5-year survival rate. On the other hand, however, in recent years, advancements in targeted therapy and immunotherapy have been significant.

Nearly 40% newly diagnosed lung cancer patients have cancers that have already metastasized. During cancer development, tumor epithelial cells transit into mesenchymal cells (EMT), and enter the blood circulation to become circulating tumor cells (CTCs). CTCs are considered to be the precursors of tumor dissemination and metastases.

### SUMMARY

Provided are methods involving the assaying of a labeled cell suspension, e.g., to detect cancer-related cells, populations thereof and/or screen for metastatic cancer. Labeled cell suspensions may be assayed to detect whether a tumor infiltrating lymphocyte (TIL) population is present in a cellular suspension of a subject. Cancer-related cell populations of interest include, e.g., those expressing one or more markers, including where the markers are members of a marker panel. Markers assayed may vary depending on the context and may include protein markers, nucleic acid markers, cell cycle markers, DNA content markers, and the like. Useful markers include one or more immune checkpoint markers and/or one or more immune cell-type markers, including where the marker(s) assayed are part of one or more panels of markers. Also provided are methods of treating a subject for a neoplasia based on the outcome of an assay of a labeled cell suspension of sample from a subject. Kits for practicing the described methods are also provided.

### BRIEF DESCRIPTION OF THE FIGURES

The invention is best understood from the following detailed description when read in conjunction with the accompanying drawings. The patent or application file contains at least one drawing executed in color. Copies of this patent or patent application publication with color drawing(s) will be provided by the Office upon request and payment of the necessary fee. It is emphasized that, according to common practice, the various features of the drawings are not to-scale. On the contrary, the dimensions of the various features are arbitrarily expanded or reduced for clarity. Included in the drawings are the following figures.

FIG. 1A-1B provides experimental workflows for solid tumor processing and analysis and circulating tumor cell (CTC) isolation and analysis.

FIG. 2 depicts cellular cancer marker expression and flow analysis.

FIG. 3 depicts cellular immune marker expression and flow analysis.

FIG. 4A-4B provide depictions of the correlation analysis of CTCs with immune checkpoint markers, immune activation markers and cancer cell markers.

FIG. 5 depicts patient sample clustering analysis.

FIG. 6 provides Table 1.

FIG. 7 provides Table 2.

FIG. 8 provides Table 3.

FIG. 9 provides Table 4.

FIG. 10 provides Table 5.

FIG. 11 provides a supplemental table as described herein.

FIG. 12 demonstrates the detection of a population of T regulatory cells isolated from lung tumor tissue that express CCR5.

FIG. 13 provides a marker panel that includes a plurality of detectable marker binding members including a detectable CCR5 binding member.

### DETAILED DESCRIPTION

Provided are methods involving the assaying of a labeled cell suspension, e.g., to detect cancer-related cells, populations thereof and/or screen for metastatic cancer. Labeled cell suspensions may be assayed to detect whether a tumor infiltrating lymphocyte (TIL) population is present in a cellular suspension of a subject. Cancer-related cell populations of interest include, e.g., those expressing one or more markers, including where the markers are members of a marker panel. Markers assayed may vary depending on the context and may include protein markers, nucleic acid markers, cell cycle markers, DNA content markers, and the like. Useful markers include one or more immune checkpoint markers and/or one or more immune cell-type markers, including where the marker(s) assayed are part of one or more panels of markers. Also provided are methods of treating a subject for a neoplasia based on the outcome of an assay of a labeled cell suspension of sample from a subject. Kits for practicing the described methods are also provided.

Before the present invention is described in greater detail, it is to be understood that this invention is not limited to particular embodiments described, as such may, of course, vary. It is also to be understood that the terminology used herein is for the purpose of describing particular embodiments only, and is not intended to be limiting, since the scope of the present invention will be limited only by the appended claims.

Where a range of values is provided, it is understood that each intervening value, to the tenth of the unit of the lower limit unless the context clearly dictates otherwise, between the upper and lower limit of that range and any other stated or intervening value in that stated range, is encompassed within the invention. The upper and lower limits of these smaller ranges may independently be included in the smaller ranges and are also encompassed within the invention, subject to any specifically excluded limit in the stated range. Where the stated range includes one or both of the limits, ranges excluding either or both of those included limits are also included in the invention.

Certain ranges are presented herein with numerical values being preceded by the term “about.” The term “about” is used herein to provide literal support for the exact number that it precedes, as well as a number that is near to or approximately the number that the term precedes. In determining whether a number is near to or approximately a specifically recited number, the near or approximating unrecited number may be a number which, in the context in which it is presented, provides the substantial equivalent of the specifically recited number.

Unless defined otherwise, all technical and scientific terms used herein have the same meaning as commonly understood by one of ordinary skill in the art to which this invention belongs. Although any methods and materials similar or equivalent to those described herein can also be used in the practice or testing of the present invention, representative illustrative methods and materials are now described.

All publications and patents cited in this specification are herein incorporated by reference as if each individual publication or patent were specifically and individually indicated to be incorporated by reference and are incorporated herein by reference to disclose and describe the methods and/or materials in connection with which the publications are cited. The citation of any publication is for its disclosure prior to the filing date and should not be construed as an admission that the present invention is not entitled to antedate such publication by virtue of prior invention. Further, the dates of publication provided may be different from the actual publication dates which may need to be independently confirmed.

It is noted that, as used herein and in the appended claims, the singular forms “a”, “an”, and “the” include plural referents unless the context clearly dictates otherwise. It is further noted that the claims may be drafted to exclude any optional element. As such, this statement is intended to serve as antecedent basis for use of such exclusive terminology as “solely,” “only” and the like in connection with the recitation of claim elements, or use of a “negative” limitation.

As will be apparent to those of skill in the art upon reading this disclosure, each of the individual embodiments described and illustrated herein has discrete components and features which may be readily separated from or combined with the features of any of the other several embodiments without departing from the scope or spirit of the present invention. Any recited method can be carried out in the order of events recited or in any other order which is logically possible.

While the apparatus and method has or will be described for the sake of grammatical fluidity with functional explanations, it is to be expressly understood that the claims, unless expressly formulated under 35 U.S.C. § 112, are not to be construed as necessarily limited in any way by the construction of “means” or “steps” limitations, but are to be accorded the full scope of the meaning and equivalents of the definition provided by the claims under the judicial doctrine of equivalents, and in the case where the claims are expressly formulated under 35 U.S.C. § 112 are to be accorded full statutory equivalents under 35 U.S.C. § 112.

### Methods

As summarized above, methods of the present disclosure may include detecting one or more cancer related cell types and/or cancer related cell populations, including tumor infiltrating lymphocyte (TIL) cell types and/or TIL populations, in a cellular suspension. Such detection may involve assaying for the expression of various markers on the cell type(s) and/or the cells of the cell population(s). Types of markers that may be assayed include but are not limited to e.g., protein markers, nucleic acid (e.g., DNA or RNA (e.g., mRNA) markers, cell cycle markers, DNA content markers, and the like. Useful markers that may be assayed may include immune checkpoint markers, immune cell-type markers, immune activation markers, adhesion-related markers, mesenchymal cell markers, epithelial cell markers, and combinations thereof.

Makers expressed by a cell may be assayed at the protein or mRNA level. For example, immune checkpoint markers, immune cell-type markers, immune activation markers, adhesion-related markers, mesenchymal cell markers, epithelial cell markers may be assayed at the protein or mRNA level. Assaying at the protein level may include e.g., detecting the presence and/or quantifying the amount of the protein expressed by the cell, e.g., by contacting the cells with a binding member specific for the protein marker. Assaying at the mRNA level may include e.g., detecting the presence and/or quantifying the amount of mRNA encoding the marker that is expressed by the cell, e.g., by contacting the cells with a binding member specific for the mRNA marker. Useful binding members for detecting and/or quantifying the amount of protein markers and/or mRNA markers are described in more detail below and include but are not limited to e.g., antibodies and nucleic acids probes. In the following, where detection/quantification of a particular protein marker using a binding member for the proteins marker is described, such may be adapted to use a mRNA binding member for detection/quantification of the mRNA encoding the protein marker. Similarly, where detection/quantification of a particular mRNA marker using a binding member for the mRNA marker is described, such may be adapted to use a protein binding member for detection/quantification of the protein encoded by the mRNA marker.

Cellular suspensions of interest, which may be subjected to a cancer-related cell type detection procedure, will vary and may include cellular suspensions obtained or prepared from neoplasia tissue and/or tissue suspected of containing a neoplasia. For example, in some instances, a cellular suspension may be obtained or prepared from a cancerous tissue, such as a tumor, and subjected to a cancer-related cell type detection procedure, e.g., to detect one or more populations of TILs expressing various markers, such as immune checkpoint markers, immune cell-type markers, and/or combinations thereof. Tissues and samples, including cancerous tissues and samples, from which cellular suspensions may be obtained or prepared include but are not limited to, e.g., biological samples, biopsy tissues, etc., which are described in more detail below.

As summarized above, cancer related cells and/or cancer related cell populations thereof, including e.g., TIL cells and/or populations thereof, which may be detected in the herein described methods may express an immune checkpoint marker, an immune cell-type marker or a combination thereof. Such markers are described in more detail below. In some instances, a detected cell or population of cells expresses both an immune checkpoint marker and an immune cell-type marker. In some instances, a cell and/or population thereof may express two or more markers. For example, a cell and/or population thereof may express multiple immune checkpoint markers, including but not limited 3 or more, 4 or more, 5 or more, 6 or more, 7 or more, 8 or more, 9 or more, 10 or more, etc. In some instances, a cell and/or population thereof may express multiple immune cell-type markers, including but not limited 3 or more, 4 or more, 5 or more, 6 or more, 7 or more, 8 or more, 9 or more, 10 or more, etc.

In some instances, a cell and/or population thereof may express a combination of multiple immune checkpoint markers and multiple immune cell-type markers. For example, a cell and/or population thereof may express 2 or more immune checkpoint markers and 2 or more immune cell-type markers, including e.g., where the cell and/or population thereof expresses any combination of 2 or more, 3 or more, 4 or more, 5 or more, 6 or more, 7 or more, 8 or more, 9 or more, 10 or more, etc., immune checkpoint markers and immune cell-type markers.

As used herein, the term “immune checkpoint marker” generally refers to markers, e.g., proteins, or mRNAs encoding such proteins, which are components of immune signaling pathways that regulate immune activation. Immune checkpoint proteins may play a role in maintaining immune homeostasis and/or preventing autoimmunity, e.g., by stimulating or preventing immune activation. Useful immune checkpoint markers include but are not limited to e.g., programmed cell death 1 (PD-1), T-cell immunoglobulin mucin receptor 3 (TIM-3), lymphocyte-activation gene-3 (LAG-3) and cytotoxic T-lymphocyte associated protein 4 (CTLA-4).

As used herein, the term “immune cell-type marker” generally refers to markers, e.g., proteins, or mRNAs encoding such proteins, which identify, define or differentiate (based on their presence or absence) one or more immune cell types, or populations thereof, of the immune cell lineage at one or more points along the cell lineage. For example, an immune cell-type marker may differentiate a myeloid cell from a lymphoid cell, different myeloid cell types from each other, different lymphoid cell types (e.g., T lymphocytes, B lymphocytes, NK cells, T regulatory (Treg) cells, etc.) from each other, a progenitor cell from a committed progenitor, a progenitor cell from a differentiated cell type, etc. Accordingly, in some instances, markers may be referred to with respect to the cell population, or subpopulation, that the marker differentiates. For example, a marker may be identified as a lymphoid marker, a T lymphocyte marker, a B lymphocyte marker, a NK cell marker, a Treg marker, or the like. In some instances, immune cell-type markers may identify a specific population or subpopulation of immune cells defined by the expression of the marker. For example, detecting CD4 may facilitate the identification of a population of CD4+ T cells, detecting CCR5 may facilitate the identification of a population of CCR5+ immune cells, and the like. Useful immune cell-type markers include but are not limited to e.g., CD3, CD4, CD8, CD16, CD19, CD25, CD56, CD127 and CCR5.

Various combinations of immune checkpoint markers and immune cell-type markers may find use in detecting cells and/or cell populations assayed in the present methods. Such combinations may include: PD-1, with or without one or more additional immune checkpoint markers, in combination with one or more immune cell-type markers; or PD-1, with or without one or more immune cell-type markers, in combination with one or more additional immune checkpoint markers. Non-limiting examples of such useful combinations may include, e.g., PD-1 in combination with CTLA-4; PD-1 in combination with CTLA-4 and CD4; PD-1 in combination with CTLA-4 and CD8; PD-1 in combination with CD4; PD-1 in combination with TIM-3; PD-1 in combination with TIM-3 and CD4; PD-1 in combination with LAG-3 and CD4; and the like.

In some instances, useful marker combinations may include: TIM-3, with or without one or more additional immune checkpoint markers, in combination with one or more immune cell-type markers; or TIM-3, with or without one or more immune cell-type markers, in combination with one or more additional immune checkpoint markers. Non-limiting examples of such useful combinations may include, e.g., TIM-3 in combination with CD4; TIM-3 in combination with CD4 and LAG3; TIM-3 in combination with CTLA-4; TIM-3 in combination with CTLA-4 and CD4; TIM-3 in combination with CTLA-4 and CD8; TIM-3 in combination with an NK cell marker; and the like.

In some instances, useful marker combinations may include: LAG-3, with or without one or more additional immune checkpoint markers, in combination with one or more immune cell-type markers; or LAG-3, with or without one or more immune cell-type markers, in combination with one or more additional immune checkpoint markers. Non-limiting examples of such useful combinations may include, e.g., LAG-3 in combination with CD4; LAG-3 in combination with CD4 and CTLA-4; and the like.

In some instances, useful marker combinations may include: CTLA-4, with or without one or more additional immune checkpoint markers, in combination with one or more immune cell-type markers; or CTLA-4, with or without one or more immune cell-type markers, in combination with one or more additional immune checkpoint markers. Non-limiting examples of such useful combinations may include, e.g., any one of the above described CTLA-4 containing combinations, and the like.

In some instances, useful marker combinations may include: CCR5, with or without one or more additional immune cell-type markers, in combination with one or more immune checkpoint markers; or CCR5, with or without one or more immune checkpoint markers, in combination with one or more additional immune cell-type markers. Non-limiting examples of such useful combinations may include, e.g., CCR5 combined with any one or more of the herein described immune checkpoint markers and/or any one or more of the herein described immune cell-type markers, including CCR5 combined with any of the herein described marker combinations, such as but not limited to e.g., those combinations specific set forth above.

Methods of the present disclosure may also include assaying a cellular suspension to determine whether two or more different cell types and/or two more different cell populations are present in the cellular suspension. For example, in some instances, methods of the present disclosure may include assaying a cellular suspension to determine whether two or more different TIL populations are present in the cellular suspension. Where the subject methods include assaying to detect two or more cell types and/or two or more cell populations, various combinations of cell types and/or cell populations may be employed. For example, the method may include assaying for a first population, which expresses a first combination of one or more immune checkpoint markers and/or one or more immune cell-type markers, and a second population, which expresses a second combination of one or more immune checkpoint markers and/or one or more immune cell-type markers.

Two or more cell populations assayed for in a subject method may or may not share one or more immune checkpoint markers in common. Put another way, two populations assayed for in a subject method may or may not express the same immune checkpoint marker, including e.g., where the two or more populations do or do not commonly express, e.g., PD-1, TIM-3, LAG-3, and/or CTLA-4.

In addition, two or more cell populations assayed for in a subject method may or may not share one or more immune cell-type markers in common. Put another way, two populations assayed for in a subject method may or may not express the same immune cell-type marker, including e.g., where the two or more populations do or do not commonly express, e.g., CD3, CD4, CD8, CD16, CD19, CD25, CD56, CD127 and/or CCR5.

Marker combinations of the herein described methods may, in some instances, include one or more immune activation markers. As used herein, the term “immune activation marker” generally refers to makers, e.g., proteins, or mRNAs encoding such proteins, expressed or upregulated by activated immune cells, e.g., in response to signaling through an immune activation signaling pathway. Cells that may be activated in immune signaling will vary and may include e.g., antigen presenting cells (i.e., APCs, such as dendritic cells, macrophages, B cells, and the like), T cells, NK cells, etc. Useful immune activation markers include but are not limited to e.g., Human Leukocyte Antigen-antigen D Related (HLA-DR).

Marker combinations of the herein described methods may, in some instances, include or exclude one or more adhesion-related markers. As used herein, the term “adhesion-related marker” generally refers to markers, e.g., proteins or mRNAs encoding such proteins, expressed by a cell to regulate attachment of the cell to neighboring cells (e.g., cell-cell interactions) and/or components of the cellular environment, such as e.g., extra cellular matrix (ECM). Accordingly, adhesion-related markers may be expressed and/or upregulated in cells that maintain or have increased adherence to surrounding cells and/or ECM and not expressed and/or downregulated in migratory cell types. Adhesion-related markers may, depending on the context, be expressed in cells with decreased attachment to neighboring cells and/or components of the cellular environment, e.g., expressed and/or upregulated in migratory cell types and not expressed and/or downregulated in cells that maintain or have increased adherence to surrounding cells and/or ECM. Adhesion-related markers may include markers involved in the engagement of one cell type with another, such as the engagement of immune cells with cancer cells, and thus, in some instances, certain adhesion-related-markers may be referred to as engagement markers. Useful adhesion-related markers include but are not limited to e.g., E cadherin, CD44 and CD103.

In some instances, useful marker combinations may include the expression of a first adhesion-related marker and the absence or undetectable expression of a second adhesion-related marker. For example, in some instances, a useful marker combination may include e.g., where the subject cell and/or cell population expresses CD44 and does not express a detectable level of E cadherin, i.e., the cell or population thereof is CD44 positive and E cadherin negative. In some instances, useful marker combinations may include the expression of two or more adhesion-related markers, including but not limited to e.g., expression of two or more of the adhesion-related markers described herein. Marker combinations of the herein described methods may, in some instances, include or exclude one or more mesenchymal or epithelial cell markers. As used herein, the term “mesenchymal cell marker” generally refers to markers, e.g., proteins, or mRNAs encoding such proteins, expressed by mesenchymal cells, i.e., cells of the mesenchyme, a diffuse network of cells giving rise to connective tissues, blood and blood vessels, the lymphatic system, and cells of other systems. Useful mesenchymal cell markers include but are not limited to e.g., vimentin, and the like. Useful mesenchymal markers include markers that, when absent in a cell population, facilitate the identification of the cell population as mesenchymal cells. In other words, a cell population may be identified as mesenchymal cells based on a lack of labeling of the cells by a detectable binding member to a marker that differentiates mesenchymal cells from other types of cells, such as epithelial cells. Useful markers for such purposes may include but are not limited to e.g., epithelial cell markers, such as but not limited to e.g., cytokeratin markers, such as e.g., Pan CK (i.e., pan cytokeratin). In some instances, a subject marker combination may include two or more mesenchymal cell markers, including but not limited to e.g., a combination including Pan CK and vimentin, including e.g., where cells may be identified as mesenchymal cells based on the presence and/or absence of one or more markers of the combination (e.g., Pan CK(−) and vimentin(+)). In some instances, cells may be identified as transitioning from epithelial to mesenchymal (i.e., undergoing epithelial-to-mesenchymal transition, EMT) through the expression of both a mesenchymal marker and an epithelial marker, including but not limited to e.g., Pan CK(+) and vimentin(+) EMT cells.

Useful marker combinations may include a combination of one or more markers, where each marker may be present or absent, from multiple of the marker categories described above, including e.g., a combination that includes one or more adhesion-related markers and one more immune activation markers; a combination that includes one or more adhesion-related markers and one more immune-cell type markers; a combination that includes one or more immune cell-type markers and one more immune activation markers; a combination that includes one or more adhesion-related markers, one more immune activation markers, and one or more immune-cell type markers; etc. For example, in some instances, useful marker combinations may include e.g., CD103 and HLA-DR; CD103, HLA-DR and a NK cell marker; and the like.

In some instances, useful combinations may include the simultaneous expression of two or more markers. Useful combinations of simultaneously expressed markers may include e.g., the simultaneous expression of an adhesion-related maker and an immune activation maker. For example, in some instances, useful marker combinations may include but are not limited to e.g., CD103 simultaneously expressed with HLA-DR. Simultaneous expression of markers will generally refer to two or more markers expressed in and/or by the same cell at the same time.

Detection of cancer-related cell types and/or populations thereof, such as TIL populations, in the instant methods may indicate the presence of a metastatic condition in the subject. For example, in some instances, through detection of a population of cancer-related cells in a sample from the subject, the methods of the present disclosure may indirectly detect and/or predict the presence of a metastatic cancer in the subject. Correspondingly, through detecting an absence of a population of cancer-related cells in a sample from the subject, the methods of the present disclosure may indirectly detect and/or predict the absence of a metastatic cancer in the subject.

As such, indirect detection and/or prediction of the presence or absence of metastatic cancer in a subject may be based on one or more cytometrically detected parameters employed to assay for one or more cancer-related cell types and/or populations thereof in a sample from the subject, such as e.g., one or more TIL cell populations that express a marker or combination of markers, e.g., as described herein. In some instances, the size of a detected cancer-related cell population may be indicative of the extent of cancer metastasis in the subject. In some instances, the number of cancer-related cell populations detected may be indicative of the extent of cancer metastasis in the subject.

Detection of cancer-related cell types and/or populations thereof, such as TIL populations, in the instant methods may indicate the presence of circulating tumor cells (CTCs) in the subject. For example, in some instances, through detection of a population of cancer-related cells in a sample from the subject, the methods of the present disclosure may indirectly detect and/or predict the presence of CTCs in the subject. Correspondingly, through detecting an absence of a population of cancer-related cells in a sample from the subject, the methods of the present disclosure may indirectly detect and/or predict the absence of CTCs in the subject. As such, indirect detection and/or prediction of the presence or absence of CTCs in a subject may be based on one or more cytometrically detected parameters employed to assay for one or more cancer-related cell types and/or populations thereof in a sample from the subject, such as e.g., one or more TIL cell populations that express a marker or combination of markers, e.g., as described herein. In some instances, the size of a detected cancer-related cell population may be indicative of the amount of CTCs in the subject. In some instances, the number of cancer-related cell populations detected may be indicative of the amount of CTCs in the subject.

In some instances, through detection of a population of cancer-related cells in a sample from the subject, the methods of the present disclosure may indirectly detect and/or predict the absence of clinically relevant metastatic cancer and/or CTCs in the subject. Put another way, detection of a particular cancer-related cell population may inversely correlate with the presence of metastatic cancer and/or CTCs such that detection of the population indicates that the presence of metastatic cancer and/or CTCs is unlikely. As a non-limiting example, the presence of a cancer-related cell population having particular expression of one or more adhesion-related markers (e.g., a population that expresses CD44 and does not express E Cadherin) may be inversely correlated with the presence in metastatic disease and CTCs, such that detecting the population indirectly indicates a lack of metastatic disease and CTCs in the subject. Correspondingly, through detecting an absence of a population of cancer-related cells in a sample from the subject, the methods of the present disclosure may indirectly detect and/or predict the presence of a metastatic cancer in the subject.

### Samples and Cell Suspensions

As summarized above, methods involving the assaying of a labeled cell suspension are provided. Accordingly, the present methods may involve obtaining and/or providing a cell suspension or a labeled cell suspension. Obtained and/or provided cell suspensions may be labeled (i.e., previously labeled) or may be labeled in the described method, e.g., by contacting an unlabeled cell suspension with one or more labeling reagents.

Methods of the present disclosure may include, in some instances, producing the cell suspension. Cell suspensions may be produced from a variety of sources including but not limited to e.g., biological samples that contain cells, such as but not limited to e.g., biopsy specimens, neoplasia samples, and the like.

A “biological sample” encompasses a variety of sample types obtained from an individual and can be used in a diagnostic or monitoring assay. The definition encompasses blood and other liquid samples of biological origin, solid tissue samples such as a biopsy specimen or tissue cultures or cells derived therefrom and the progeny thereof. The term “biological sample” includes urine, saliva, cerebrospinal fluid, interstitial fluid, ocular fluid, synovial fluid, blood fractions such as plasma and serum, and the like; however, in relationship to the present disclosure biological samples will generally include cells and, thus, may include solid tissue samples, such as solid tissue biopsies, semi-solid tissues and biopsies thereof, liquid biopsies, cellular samples, such as aspirated cellular samples, and the like.

The herein described methods are applicable to various neoplasia samples where a neoplasia sample may include a sample of any neoplastic (i.e., abnormally growing) tissue or cell population or cell. Abnormal tissue growth may be determined by a variety of means including e.g., by comparing the growth of the subject tissue to the growth of an appropriate normal or healthy tissue. Neoplasms include benign neoplasms, in situ neoplasms, malignant neoplasms, and neoplasms of uncertain or unknown behavior. Malignant neoplasms include cancer.

In some instances, a biological sample from which a cell suspension is prepared may be a sample obtained from a subject having a cancer or suspected of having a cancer. For example, the present methods may be applied to a sample from a subject having or suspected of having any one or more of various different cancers including but not limited to e.g., Acute Lymphoblastic Leukemia (ALL), Acute Myeloid Leukemia (AML), Adrenocortical Carcinoma, AIDS-Related Cancers (e.g., Kaposi Sarcoma, Lymphoma, etc.), Anal Cancer, Appendix Cancer, Astrocytomas, Atypical Teratoid/Rhabdoid Tumor, Basal Cell Carcinoma, Bile Duct Cancer (Extrahepatic), Bladder Cancer, Bone Cancer (e.g., Ewing Sarcoma, Osteosarcoma and Malignant Fibrous Histiocytoma, etc.), Brain Stem Glioma, Brain Tumors (e.g., Astrocytomas, Central Nervous System Embryonal Tumors, Central Nervous System Germ Cell Tumors, Craniopharyngioma, Ependymoma, etc.), Breast Cancer (e.g., female breast cancer, male breast cancer, childhood breast cancer, etc.), Bronchial Tumors, Burkitt Lymphoma, Carcinoid Tumor (e.g., Childhood, Gastrointestinal, etc.), Carcinoma of Unknown Primary, Cardiac (Heart) Tumors, Central Nervous System (e.g., Atypical Teratoid/Rhabdoid Tumor, Embryonal Tumors, Germ Cell Tumor, Lymphoma, etc.), Cervical Cancer, Childhood Cancers, Chordoma, Chronic Lymphocytic Leukemia (CLL), Chronic Myelogenous Leukemia (CML), Chronic Myeloproliferative Neoplasms, Colon Cancer, Colorectal Cancer, Craniopharyngioma, Cutaneous T-Cell Lymphoma, Duct (e.g., Bile Duct, Extrahepatic, etc.), Ductal Carcinoma In Situ (DCIS), Embryonal Tumors, Endometrial Cancer, Ependymoma, Esophageal Cancer, Esthesioneuroblastoma, Ewing Sarcoma, Extracranial Germ Cell Tumor, Extragonadal Germ Cell Tumor, Extrahepatic Bile Duct Cancer, Eye Cancer (e.g., Intraocular Melanoma, Retinoblastoma, etc.), Fibrous Histiocytoma of Bone (e.g., Malignant, Osteosarcoma, etc.), Gallbladder Cancer, Gastric (Stomach) Cancer, Gastrointestinal Carcinoid Tumor, Gastrointestinal Stromal Tumors (GIST), Germ Cell Tumor (e.g., Extracranial, Extragonadal, Ovarian, Testicular, etc.), Gestational Trophoblastic Disease, Glioma, Hairy Cell Leukemia, Head and Neck Cancer, Heart Cancer, Hepatocellular (Liver) Cancer, Histiocytosis (e.g., Langerhans Cell, etc.), Hodgkin Lymphoma, Hypopharyngeal Cancer, Intraocular Melanoma, Islet Cell Tumors (e.g., Pancreatic Neuroendocrine Tumors, etc.), Kaposi Sarcoma, Kidney Cancer (e.g., Renal Cell, Wilms Tumor, Childhood Kidney Tumors, etc.), Langerhans Cell Histiocytosis, Laryngeal Cancer, Leukemia (e.g., Acute Lymphoblastic (ALL), Acute Myeloid (AML), Chronic Lymphocytic (CLL), Chronic Myelogenous (CML), Hairy Cell, etc.), Lip and Oral Cavity Cancer, Liver Cancer (Primary), Lobular Carcinoma In Situ (LCIS), Lung Cancer (e.g., Non-Small Cell, Small Cell, etc.), Lymphoma (e.g., AIDS-Related, Burkitt, Cutaneous T-Cell, Hodgkin, Non-Hodgkin, Primary Central Nervous System (CNS), etc.), Macroglobulinemia (e.g., Waldenström, etc.), Male Breast Cancer, Malignant Fibrous Histiocytoma of Bone and Osteosarcoma, Melanoma, Merkel Cell Carcinoma, Mesothelioma, Metastatic Squamous Neck Cancer with Occult Primary, Midline Tract Carcinoma Involving NUT Gene, Mouth Cancer, Multiple Endocrine Neoplasia Syndromes, Multiple Myeloma/Plasma Cell Neoplasm, Mycosis Fungoides, Myelodysplastic Syndromes, Myelodysplastic/Myeloproliferative Neoplasms, Myelogenous Leukemia (e.g., Chronic (CML), etc.), Myeloid Leukemia (e.g., Acute (AML), etc.), Myeloproliferative Neoplasms (e.g., Chronic, etc.), Nasal Cavity and Paranasal Sinus Cancer, Nasopharyngeal Cancer, Neuroblastoma, Non-Hodgkin Lymphoma, Non-Small Cell Lung Cancer, Oral Cancer, Oral Cavity Cancer (e.g., Lip, etc.), Oropharyngeal Cancer, Osteosarcoma and Malignant Fibrous Histiocytoma of Bone, Ovarian Cancer (e.g., Epithelial, Germ Cell Tumor, Low Malignant Potential Tumor, etc.), Pancreatic Cancer, Pancreatic Neuroendocrine Tumors (Islet Cell Tumors), Papillomatosis, Paraganglioma, Paranasal Sinus and Nasal Cavity Cancer, Parathyroid Cancer, Penile Cancer, Pharyngeal Cancer, Pheochromocytoma, Pituitary Tumor, Pleuropulmonary Blastoma, Primary Central Nervous System (CNS) Lymphoma, Prostate Cancer, Rectal Cancer, Renal Cell (Kidney) Cancer, Renal Pelvis and Ureter, Transitional Cell Cancer, Retinoblastoma, Rhabdomyosarcoma, Salivary Gland Cancer, Sarcoma (e.g., Ewing, Kaposi, Osteosarcoma, Rhabdomyosarcoma, Soft Tissue, Uterine, etc.), Sezary Syndrome, Skin Cancer (e.g., Childhood, Melanoma, Merkel Cell Carcinoma, Nonmelanoma, etc.), Small Cell Lung Cancer, Small Intestine Cancer, Soft Tissue Sarcoma, Squamous Cell Carcinoma, Squamous Neck Cancer (e.g., with Occult Primary, Metastatic, etc.), Stomach (Gastric) Cancer, T-Cell Lymphoma, Testicular Cancer, Throat Cancer, Thymoma and Thymic Carcinoma, Thyroid Cancer, Transitional Cell Cancer of the Renal Pelvis and Ureter, Ureter and Renal Pelvis Cancer, Urethral Cancer, Uterine Cancer (e.g., Endometrial, etc.), Uterine Sarcoma, Vaginal Cancer, Vulvar Cancer, Waldenström Macroglobulinemia, Wilms Tumor, and the like.

Samples useful in the herein described methods may be samples obtained from a primary tumor, e.g., from a biopsy or surgical resection, or a non-tumor tissue. Non-tumor tissues may be assessed for various reasons including but not limited to for cancer surveillance, as a control, etc. Both solid and fluid non-tumor samples may be assessed. Useful solid tissues that may be assessed include but are not limited to e.g., tissue adjacent to an existing cancer (e.g., skin tissue, lung tissue, breast tissue, etc.), lymph node tissue, etc. Useful fluid samples that may be assessed include essentially any bodily fluid sample including but are not limited to e.g., blood samples, lymph fluid samples, etc.

Cancer and tumor tissues that may be assessed likewise include solid and liquid samples. For example, in the case of a hematopoietic cancer a blood sample or a bone marrow sample may be assessed. In some instances, the sample assessed according to the herein described methods is a solid tumor sample. Solid tumor samples may be obtained from a variety of different cancers, including e.g., any of those cancers listed above. In some instances, a solid tumor sample may be a cancer of an epithelial tissue or an epithelial cancer.

Epithelial cancers include carcinomas. Non-limiting examples of carcinomas include acinar carcinoma, acinic cell carcinoma, acinous carcinoma, adenocystic carcinoma, adenoid cystic carcinoma, adenosquamous carcinoma, adnexal carcinoma, adrenocortical carcinoma, alveolar carcinoma, ameloblastic carcinoma, apocrine carcinoma, basal cell carcinoma, bronchioloalveolar carcinoma, bronchogenic carcinoma, cholangiocellular carcinoma, chorionic carcinoma, clear cell carcinoma, colloid carcinoma, cribriform carcinoma, ductal carcinoma in situ, embryonal carcinoma, carcinoma en cuirasse, endometrioid carcinoma, epidermoid carcinoma, carcinoma ex mixed tumor, carcinoma ex pleomorphic adenoma, follicular carcinoma of thyroid gland, hepatocellular carcinoma, carcinoma in situ, intraductal carcinoma, Hürthle cell carcinoma, inflammatory carcinoma of the breast, large cell carcinoma, invasive lobular carcinoma, lobular carcinoma, lobular carcinoma in situ (LCIS), medullary carcinoma, meningeal carcinoma, Merkel cell carcinoma, mucinous carcinoma, mucoepidermoid carcinoma, nasopharyngeal carcinoma, non-small cell carcinoma, non-small cell lung carcinoma (NSCLC), oat cell carcinoma, papillary carcinoma, renal cell carcinoma, scirrhous carcinoma, sebaceous carcinoma, carcinoma simplex, signet-ring cell carcinoma, small cell carcinoma, small cell lung carcinoma, spindle cell carcinoma, squamous cell carcinoma, terminal duct carcinoma, transitional cell carcinoma, tubular carcinoma, verrucous carcinoma, and the like.

In some instances, the methods described herein find use in detecting whether a cancer-related cell type and/or a population thereof is present in an epithelial tumor sample, including e.g., an epithelial lung cancer tumor, an epithelial breast cancer tumor, etc. In some instances, the methods described herein find use in detecting the presence or absence of a cancer-related cell type and/or population thereof in a non-small cell lung cancer (NSCLC) tumor. In some instances, the epithelial tumor is a squamous cell carcinoma, an adenocarcinoma or an adenosquamous and the detected cell(s) include squamous cell carcinoma cells, adenocarcinoma cells or adenosquamous carcinoma cells.

Samples containing cancer-related cells or suspected of containing cancer-related cells may be obtained using any convenient sample collection method, including but not limited to those biopsy methods for obtaining solid tissue biopsies and biopsy aspirates. In some instances, a sample may be obtained as part of a separate medical procedure performed for a purpose other than obtaining the sample, including but not limited to a surgical procedure. In other instances, a sample may be obtained independently, e.g., not as part of a separate medical procedure. Sample collection methods will vary and will depend upon, e.g., whether the collection is or is not performed as part of an additional medical procedure, the particular type of sample to be obtained, the primary purpose for obtaining the sample and/or the method by which the sample is to be processed and/or analyzed.

Samples used in the methods of the present disclosure may be collected by any convenient means. In some instances, a sample is prepared from a biopsy. Depending on the type of cancer and/or the type of biopsy performed the sample may be prepared from a solid tissue biopsy or a liquid biopsy.

In some instances, a sample may be prepared from a surgical biopsy. Any convenient and appropriate technique for surgical biopsy may be utilized for collection of a sample to be assessed according to the methods described herein including but not limited to, e.g., excisional biopsy, incisional biopsy, wire localization biopsy, and the like. In some instances, a surgical biopsy may be obtained as a part of a surgical procedure which has a primary purpose other than obtaining the sample, e.g., including but not limited to tumor resection, mastectomy, lymph node surgery, axillary lymph node dissection, sentinel lymph node surgery, and the like.

In some instances, a sample may be obtained by a needle biopsy. Any convenient and appropriate technique for needle biopsy may be utilized for collection of a sample to be analyzed according to the methods described herein including but not limited to, e.g., fine needle aspiration (FNA), core needle biopsy, stereotactic core biopsy, vacuum assisted biopsy, and the like.

FNA biopsy may be performed on both palpable and non-palpable lesions and involves the introduction of a small-gauge needle, e.g., ranging from 18 to 25 gauge, into the mass or suspected area and the extraction of cellular material. Whether FNA is performed with or without co-imaging may vary and will depend on various factors including whether the lesion is palpable. In instances where FNA is performed with co-imaging the technique may be referred to as image-guided FNA and may include but is not limited to radiological imaging techniques such as ultrasound, computed tomography (CT), fluoroscopy, mammography, MRI, and the like. FNA techniques, and variations thereof, useful in collecting samples as described herein will vary and selection of specific techniques will depend on various factors including but not limited to, e.g., the characteristics of the subject, the characteristics of the particular detected lesion, the analysis procedure, etc. Variations of such FNA techniques include but are not limited to, e.g., the open-ended needle (i.e., the “French technique”), the negative pressure technique, imaging-guided FNA, and the like. As such, particular FNA techniques may or may not include negative suction. For example, in the French technique FNA short, rapid strokes within the lesion cause dislodgement of cells and allow effective collection within the needle via capillary action without the need for negative suction. In some instances, e.g., when excess fluid (e.g., of a cystic lesion), a syringe with plunger removed may be employed in collecting a sample by FNA. In some instances, negative pressure may be utilized to draw the sample into a syringe. In some instances, a syringe holder or aspiration gun or aspiration handle may be used.

Core needle biopsy may be performed on both palpable and non-palpable lesions and involves the introduction of a hollow core needle into the mass or suspected area and the extraction of cellular material. Whether core needle biopsy is performed with or without co-imaging may vary and will depend on various factors including whether the lesion is palpable. In instances where core needle biopsy is performed with co-imaging the technique may be referred to as image-guided core needle biopsy or stereotactic core needle biopsy and may include but is not limited to radiological imaging techniques such as ultrasound, computed tomography (CT), fluoroscopy, mammography, MRI, and the like. Variations of such core needle biopsy techniques include but are not limited to, e.g., vacuum-assisted core biopsy, imaging-guided core biopsy, and the like. As such, particular core needle biopsy techniques may or may not include an incision made in the skin prior to insertion of the core biopsy needle. For example, in the vacuum-assisted core biopsy a small cut is made and a hollow probe is put through the cut and guided to the lesion site and then a cylinder of tissue is then pulled into the probe by vacuum pressure. In general, a core needle biopsy obtains more tissue than the described FNA technique.

In some instances, the term “needle biopsy” may generally refer any biopsy which can be performed without anesthesia or may require only local anesthesia and which are not considered surgical procedures. In some instances, such biopsies may utilize devices other than “needles” such as, but not limited to, those devices that may be utilized to obtain a punch biopsy, e.g., a skin punch biopsy. Such devices include but are not limited to, e.g., those devices used in the collection of skin punch biopsies.

According to the particular biopsy method employed and depending on the specifics of a particular subject and/or a subject's particular lesion one biopsy or multiple biopsies may be performed. For example, in some instances, a single biopsy, e.g., a single FNA biopsy or a single core needle biopsy, may be performed to sufficiently sample a particular subject or a particular subject's lesion. In other instances, multiple biopsies, e.g., multiple FNA biopsies or multiple core needle biopsies, may be performed for the collection of a single sample or multiple samples from a subject or a subject's lesion. In instances where multiple biopsies are collected the actual number of biopsies will vary depending on the particular subject and/or the particular lesion or lesions of the subject and, as such, may range from 2 to 10 or more biopsies, including but not limited to, e.g., 2 biopsies, 3 biopsies, 4 biopsies, 5 biopsies, 6 biopsies, 7 biopsies, 8 biopsies, 9 biopsies, 10 biopsies, etc. Multiple biopsies may be collected in a co-timely manner or may be collected over a pre-determined period of time, e.g., as part of a surveillance protocol.

Samples collected according to the methods described herein may be solid, semi-solid, or liquid samples. For example, in some instances, by nature of the collection technique utilized, e.g., techniques that cause the dissociation or aspiration of cells, the collected sample may be a liquid sample upon collection. In other instances, by nature of the collection technique utilized, e.g., surgical collection or core sample collection, the collected sample may be a solid or semi-solid sample upon collection. In embodiments where the collected sample is a solid or semi-solid sample the cells of the sample may be dissociated to form a liquid sample following collection. Methods of dissociating solid and semi-solid tissue samples include but are not limited to mechanical dissociation, chemical dissociation, enzymatic dissociation, and combinations thereof.

In some instances, solid tumor samples may be subjected to mechanical homogenization. Any convenient method of mechanical homogenization may find use preparing a solid tissue sample for downstream steps including but not limited homogenization performed using a commercially available homogenization device including e.g., those available from IncellDx (Menlo Park, Calif.), such as e.g., those provided with the incellPREP (IncellDx, Inc) kit, Claremont BioSolutions (Upland, Calif.) including e.g., the microHomogenizer (Claremont BioSolutions), the microDisruptor (Claremont BioSolutions), and the like. Mechanical homogenization may be performed in any suitable solution, including e.g., a buffer. In some instances, mechanical homogenization may be combined with chemical or enzymatic homogenization. In some instances, a fixation reagent is added during homogenization. In some instances, a fixation reagent is added following, including immediately following, homogenization. Fixation reagents, described in more detail below, that may be added following homogenization include but are not limited to e.g., the incellPREP (IncellDx, Inc). In some instances, a fixation solution may be a combination fixation/permeabilization reagent. In some instances, a fixation solution may be solely a fixation solution.

Homogenization or dissociation of cellular samples may or may not employ enzymes to facilitate and/or assist in producing a homogenized or dissociated cell suspension. As such, employed methods of homogenization or dissociation may be enzymatic or non-enzymatic. Accordingly, the methods of the present disclosure may include or specifically exclude contacting a sample, including a liquid sample or a solid sample such as a tissue, with an enzyme for homogenizing or dissociating the cells of the sample.

Upon collection or preparation of the sample, e.g., dissociation or homogenization, the cells of the resultant liquid cell suspension of may be fixed. As such, aspects of the methods may include fixing the cells of the suspension by contacting the sample with a suitable fixation reagent. Fixation reagents of interest are those that fix the cells at a desired time-point. Any convenient fixation reagent may be employed, where suitable fixation reagents include, but are not limited to mildly cross-linking agents. In some instances, a mildly cross-linking agent may be a formaldehyde-based fixative including but not limited to e.g., formaldehyde, paraformaldehyde, formaldehyde/acetone, IncellMAX (also known as IncellFP; IncellDx, Inc), etc. In some instances, an alcohol-based fixative may be employed including but not limited to e.g., methanol/acetone, ethanol, etc. In some instances, formaldehyde-based fixatives may be used at a final concentration of about 1 to 2%.

Samples used in the methods of the present disclosure are assayed cytometrically. Accordingly, in some instances, a sample may be processed to generate a cell suspension suitable for cytometric assays. Processing to generate samples suitable for cytometric assays may include or exclude e.g., any individual step or combination of the steps described above including e.g., homogenization, dissociation, fixation, permeabilization, combination fixation/permeabilization, etc., where appropriate. The amount of processing required will depend on various factors including the source of the sample, where solid tissue samples will generally require more processing that a liquid sample. For example, processing of a liquid sample, e.g., hematopoietic sample, may not require homogenization or dissociation and thus may only require fixation and/or permeabilization as desired.

In some instances, the cells in the sample are permeabilized by contacting the cells with a permeabilizing reagent. Permeabilizing reagents of interest are reagents that allow marker binding members, e.g., as described in greater detail herein, to access to the intracellular environment. Various useful permeabilizing reagents may be employed, where suitable reagents include, but are not limited to: mild detergents, such as Triton X-100, NP-40, saponin, etc.; methanol, and the like.

In some instances, methods of the present disclosure may include producing a labeled cell suspension. Labeled cells suspensions may be produced from obtained cell suspensions or a part of a procedure of producing a cell suspension, e.g., from a sample such as a solid tissue biopsy, a cellular aspirate, etc. Methods of producing a labeled cell suspension may vary and may include, e.g., contacting a cellular suspension with one or more binding members to label the cells of the cellular suspension with the one or more binding members. Useful binding members will generally be specific for a marker and may be detectable, including e.g., where the binding member is detectably labeled, as described in more detail below.

Contacting of a cell suspension with a binding member may be performed individually, i.e., where the cells of the suspension are labeled with a binding member to a single marker essentially one at a time, or in parallel, i.e., where the cells of the suspension are labeled with binding members to two or more markers at essentially the same time. Accordingly, as summarized above, in some instances labeling of a cell suspension may be performed by contacting the cell suspension with one or more panels of markers where each panel includes binding members to a plurality of different markers for simultaneous labeling of the cells with all the markers of the panel. In some instances, a cell suspension may be simultaneously contacted with binding members to at least two different markers and up to 25 or 30 or more different markers, including but not limited to e.g., at least 5 different markers, at least 6 different markers, at least 7 different markers, at least 8 different markers, at least 9 different markers, at least 10 different markers, at least 11 different markers, at least 12 different markers, at least 13 different markers, at least 14 different markers, at least 15 different markers, at least 16 different markers, at least 17 different markers, at least 18 different markers, at least 19 different markers, at least 20 different markers, etc.

In some instances, contacting of a cell suspension with a binding member may include contacting the cell suspension with an immune checkpoint panel that includes detectable binding members to a plurality of different immune checkpoint markers, including but not limited to e.g., at least 2 different immune checkpoint markers, at least 3 different immune checkpoint markers, at least 4 different immune checkpoint markers, etc. In some instances, the binding members of the plurality may be differently detectable, e.g., through the use of different labels (e.g., different fluorochromes), allowing for each different immune checkpoint marker to individually assessed when assayed.

In some instances, contacting of a cell suspension with a binding member may include contacting the cell suspension with an immune cell-type panel that includes detectable binding members to a plurality of different immune cell-type markers, including but not limited to e.g., at least 2 different immune cell-type markers, at least 3 different immune cell-type markers, at least 4 different immune cell-type markers, at least 5 different immune cell-type markers, at least 6 different immune cell-type markers, at least 7 different immune cell-type markers, at least 8 different immune cell-type markers, at least 9 different immune cell-type markers, at least 10 different immune cell-type markers, etc. In some instances, the binding members of the plurality may be differently detectable, e.g., through the use of different labels (e.g., different fluorochromes), allowing for each different immune cell-type marker to individually assessed when assayed.

Various marker panels, including various combinations of detectable binding members, may be employed. Marker panels may, e.g., be assembled from various combinations of any of the markers described herein, including but not limited to e.g., protein markers, nucleic acid markers, cell cycle markers, DNA content markers, immune checkpoint markers, immune cell-type markers, immune activation markers, adhesion-related markers, mesenchymal cell markers, epithelial cell markers, and combinations thereof. For example, in some instances, markers specific for different immune cell-type markers and different immune checkpoint markers may be combined into an “immune marker panel”. Such immune marker panels will vary and may include e.g., at least 5 different markers in total, at least 6 different markers in total, at least 7 different markers in total, at least 8 different markers in total, at least 9 different markers in total, at least 10 different markers in total, at least 11 different markers in total, at least 12 different markers in total, at least 13 different markers in total, at least 14 different markers in total, at least 15 different markers in total, etc.

Contacting, e.g., contacting a cell of a cell suspension with a specific binding member, may be carried out by any convenient and appropriate means. In some instances, a cell of a cell suspension may be contacted with a specific binding member by adding an aliquot of the specific binding member to the cell suspension. A contacted cell suspension may be incubated and/or post-fixed as desired. Specific binding members may be contacted with cells under conditions sufficient for the labeling of the marker with the binding member. Conditions sufficient for the labeling may vary depending on the particular binding member employed. For example, antibody binding members may be contacted with cells under sufficient conditions that may include, in some instances, incubation at, above, or below, room temperature; in an appropriate medium or buffer; and with or without an appropriate blocking reagent (e.g., bovine serum albumin (BSA), fetal bovine serum (FBS), or the like).

Nucleic acid binding members may, in some instances, be contacted under sufficient conditions that may include hybridization conditions. Useful hybridization conditions include stringent hybridization conditions. The term “stringent hybridization conditions” as used herein refers to conditions that are compatible to produce binding pairs of nucleic acid marker and nucleic acid binding member of sufficient complementarity to provide for the desired level of specificity in the assay while being less compatible to the formation of binding pairs between binding members of insufficient complementarity to provide for the desired specificity. Stringent assay conditions are the summation or combination (totality) of both hybridization and wash conditions.

A “stringent hybridization” and “stringent hybridization wash conditions” in the context of nucleic acid hybridization (e.g., as in array, Southern or Northern hybridizations, in-situ hybridizations, and the like) are sequence dependent, and are different under different experimental parameters. Stringent hybridization conditions that may be used can include, e.g., hybridization in a buffer comprising 30% formamide, 5×SSC, with or without detergent (e.g., 1% SDS) at 43±1° C., or the like. Useful stringent hybridization conditions may vary and may also include hybridization in a buffer of 40% formamide with a salt (e.g., 1 M NaCl) with or without a detergent (e.g., 1% SDS) at 37° C., and a wash in 1×SSC at 45° C. Yet additional stringent hybridization conditions include hybridization at 60° C. or higher and 3×SSC or incubation at 42° C. in a solution containing 30% formamide, 1M NaCl, 0.5% sodium sarcosine, 50 mM MES, pH 6.5. Those of ordinary skill will readily recognize that alternative but comparable hybridization and wash conditions can be utilized to provide conditions of similar stringency.

The individual binding members of a marker panel may, in some instances, be individually added to a cell suspension for labeling. In other instances, all or a portion of the individual binding members of the panel may be pre-mixed, e.g., in a single tube or cocktail. In some instances, binding members may be grouped, e.g., grouped by type into two or more cocktails, including but not limited to e.g., an antibody cocktail targeting a plurality of protein markers, a nucleic acid probe cocktail targeting a plurality of mRNA markers, and the like.

Methods of the present disclosure may include contacting the sample and/or cell suspension with one or more additional reagents. Useful additional reagents will vary and may include but are not limited to e.g., DNA labeling/staining reagents, cell suspension fixation solutions, fixation reagents, buffers, diluents, and the like. Reagents employed in preparing the cell suspension for analysis may be contacted with the cells in individual steps, e.g., through the use of one or more wash and/or collection steps, such that two or more reagents are not present in solution together. Reagents employed in preparing the cell suspension for analysis may be contacted with the cells in combination, i.e., during the same step, such that the two or more reagents are present together in solution during the contacting step. As such, various means of preparing the labeled cell suspension for analysis may be employed.

### Cytometry

As summarized above, methods of the present disclosure may include assaying a labeled cell suspension. Various methods of assaying a labeled cell suspension may be employed including e.g., where the cell suspension is assayed cytometrically.

As used herein, the term “cytometrically assaying” describes the measuring of cellular parameters on a cell-by-cell basis where such measuring allows for the detection of individual cells that have, or the counting of a cell population that shares, a certain cellular parameter or set of parameters. Such parameters that may be cytologically assayed in the subject methods include, e.g., per cell expression of an immune checkpoint marker, per cell expression of an immune cell-type marker, etc.

Various methods of cytometrically assaying a labeled cell suspension may find use in the herein described methods including but not limited to e.g., flow cytometrically assaying using a flow cytometer, cell cytometrically assaying a labeled cell suspension, e.g., by using a cell cytometer, and the like. Labeled cell suspension samples may be assayed for marker expression and/or the expression of multiple markers, e.g., the markers of a marker panel. In some cases, additional cellular parameters, assayed cytometrically, may also find use in detecting neoplastic cells of the instant disclosure. Accordingly, various methods of cytometrically assaying a labeled cell suspension to measure various cellular parameters may be employed.

In some embodiments, cytometrically assaying a cellular sample may be performed using flow cytometry. Flow cytometry is a methodology using multi-parameter data for identifying and distinguishing between different particle (e.g., cell) types i.e., particles that vary from one another in terms of label (wavelength, intensity), size, etc., in a fluid medium. In flow cytometrically analyzing a sample, an aliquot of the sample is first introduced into the flow path of the flow cytometer. When in the flow path, the cells in the sample are passed substantially one at a time through one or more sensing regions, where each of the cells is exposed separately and individually to a source of light at a single wavelength (or in some instances two or more distinct sources of light) and measurements of cellular parameters, e.g., light scatter parameters, and/or marker parameters, e.g., fluorescent emissions, as desired, are separately recorded for each cell. The data recorded for each cell is analyzed in real time or stored in a data storage and analysis means, such as a computer, for later analysis, as desired.

In flow cytometry-based methods, the cells are passed, in suspension, substantially one at a time in a flow path through one or more sensing regions where in each region each cell is illuminated by an energy source. The energy source may include an illuminator that emits light of a single wavelength, such as that provided by a laser (e.g., He/Ne or argon) or a mercury arc lamp or an LED with appropriate filters. For example, light at 488 nm may be used as a wavelength of emission in a flow cytometer having a single sensing region. For flow cytometers that emit light at two distinct wavelengths, additional wavelengths of emission light may be employed, where specific wavelengths of interest include, but are not limited to: 405 nm, 535 nm, 561 nm, 635 nm, 642 nm, and the like. Following excitation of a labeled specific binding member bound to a polypeptide by an energy source, the excited label emits fluorescence and the quantitative level of the polypeptide on each cell may be detected, by one or more fluorescence detectors, as it passes through the one or more sensing regions.

In flow cytometry, in addition to detecting fluorescent light emitted from cells labeled with fluorescent markers, detectors, e.g., light collectors, such as photomultiplier tubes (or “PMT”), an avalanche photodiode (APD), etc., are also used to record light that passes through each cell (generally referred to as forward light scatter), light that is reflected orthogonal to the direction of the flow of the cells through the sensing region (generally referred to as orthogonal or side light scatter) as the cells pass through the sensing region and is illuminated by the energy source. Each type of data that is obtained, e.g., forward light scatter (or FSC), orthogonal light scatter (SSC), and fluorescence emissions (FL1, FL2, etc.), comprise a separate parameter for each cell (or each “event”).

Flow cytometers may further include one or more electrical detectors. In certain embodiments, an electrical detector may be employed for detecting a disturbance caused by a particle or cell passing through an electrical field propagated across an aperture in the path of the particles/cells. Such flow cytometers having electrical detectors will contain a corresponding electrical energy emitting source that propagates an electrical field across the flow path or an aperture through which cells are directed. Any convenient electrical field and/or combination of fields with appropriate detector(s) may be used for the detection and/or measurement of particles (or cells) passing through the field including but not limited to, e.g., a direct current electrical field, alternating current electrical field, a radio-frequency field, and the like.

Flow cytometers further include data acquisition, analysis and recording means, such as a computer, wherein multiple data channels record data from each detector for each cell as it passes through the sensing region. The purpose of the analysis system is to classify and count cells wherein each cell presents itself as a set of digitized parameter values and to accumulate data for the sample as a whole.

A particular cell subpopulation of interest may be analyzed by “gating” based on the data collected for the entire population. To select an appropriate gate, the data is plotted so as to obtain appropriate separation of subpopulations, e.g., by adjusting the configuration of the instrument, including e.g., excitation parameters, collection parameters, compensation parameters, etc. In some instances, this procedure is done by plotting forward light scatter (FSC) vs. side (i.e., orthogonal) light scatter (SSC) on a two dimensional dot plot. The flow cytometer operator then selects the desired subpopulation of cells (i.e., those cells within the gate) and excludes cells which are not within the gate. Where desired, the operator may select the gate by drawing a line around the desired subpopulation using a cursor on a computer screen. Only those cells within the gate are then further analyzed by plotting the other parameters for these cells, such as fluorescence.

Any flow cytometer that is capable of obtaining fluorescence data, e.g., as described above, may be employed. Useful flow cytometers include those utilizing various different means of flowing a cell through the sensing region substantially one at a time including, e.g., a flow cell, a microfluidics chip, etc. Non-limiting examples of flow cytometer systems of interest are those available from commercial suppliers including but not limited to, e.g., Becton-Dickenson (Franklin Lakes, N.J.), Life Technologies (Grand Island, N.Y.), Acea Biosciences (San Diego, Calif.), Beckman-Coulter, Inc. (Indianapolis, Ind.), Bio-Rad Laboratories, Inc. (Hercules, Calif.), Cytonome, Inc. (Boston, Mass.), Amnis Corporation (Seattle, Wash.), EMD Millipore (Billerica, Mass.), Sony Biotechnology, Inc. (San Jose, Calif.), Stratedigm Corporation (San Jose, Calif.), Union Biometrica, Inc. (Holliston, Mass.), Cytek Development (Fremont, Calif.), Propel Labs, Inc. (Fort Collins, Colo.), Orflow Technologies (Ketchum, Id.), handyem inc. (Québec, Canada), Sysmex Corporation (Kobe, Japan), Partec Japan, Inc. (Tsuchiura, Japan), Bay bioscience (Kobe, Japan), Furukawa Electric Co. Ltd. (Tokyo, Japan), On-chip Biotechnologies Co., Ltd (Tokyo, Japan), Apogee Flow Systems Ltd. (Hertfordshire, United Kingdom), and the like.

In some embodiments, cytometrically assaying a cellular sample may be performed using a cell cytometer. As used herein, the term “cell cytometer” (also referred to as an “imaging cytometer” or “automated imaging cytometer”) generally refers to an automated or semi-automated cell imaging device capable of imaging cells deposited on or in an imaging vessel to collect data on all or most of the cells of a sample. In cell cytometry, imaging may be performed according to a variety of different methods. In some instances, a cell cytometer may collect a widefield image at low magnification (e.g., 5×, 10×, etc.) of the cells present on or in an imaging vessel to identify the location of the cells and/or screen the cells for a particular parameter (e.g., size, shape, color, fluorescence, etc.). After identifying the location of the cells a cell cytometer may proceed to collect higher magnification (e.g., 20×, 40×, 60×, 100×, etc.) images of all or a portion of the identified cells, e.g., in a targeted manner.

In other instances, a cell cytometer may image cells present on or in an imaging vessel by scanning the imaging vessel. Scanning may be performed at low or high magnification. In some instances, scanning is performed at high magnification to capture images of all or most of the cells. In some instances, scanning is performed at low magnification to identify the location of the cells on or in the imaging vessel. After identifying the location of the cells, a cell cytometer may proceed to collect higher magnification images of all or a portion of the identified cells, e.g., in a targeted manner, or may rescan the located cells at high magnification.

The imaging vessels used in cell cytometer systems will vary. In some instances, commonly used laboratory imaging devices such as e.g., microscope slides, may serve as an imaging vessel in a cell cytometer system. In some instances, a cell cytometer imaging vessel may be specifically designed for use with a particular cell cytometer. Useful imaging vessels include but are not limited to e.g., slides (e.g., microscope slides), dishes (e.g., glass bottom imaging dishes), plates (e.g., multi-well imaging plates), etc. Imaging vessels will generally have optical properties amendable to microscopy, e.g., optical clarity, in at least a portion of the vessel. Imaging vessels may or may not have individual compartments. For example, a microscope slide utilized as an imaging vessel does not generally have individual compartments and cells deposited on a slide may be spread about the surface of the slide. Alternatively, a multi-well imaging plate utilized as an imaging vessel does have individual compartments (i.e., wells) into which one or more cells may be deposited.

Cell cytometers include an imaging component such as, e.g., an automated microscope. The imaging component of a cell cytometer may include one or more objectives of various magnification power (e.g., 5×, 10×, 20×, 40×60×, 100×, etc.) for collecting light transmitted, reflected or emitted from the object (e.g., cell) being imaged. Light collected by the objective will generally be processed through one or more dichroic mirrors, filters or lenses before being directed to an image capture device.

Suitable image capturing devices may include one or more digital cameras (including color and monochrome cameras) capable of capturing a digital image and a means of storing the digital image and/or transferring the image to attached image processing circuitry or to an attached storage device for later transfer to image processing circuitry. Suitable digital color cameras will vary and will generally include any digital camera (e.g., with one or more CCD or CMOS sensors). Suitable digital cameras include but are not limited to e.g., custom built digital cameras, consumer grade digital color cameras (e.g., consumer grade digital color cameras converted for microscopic use) and those digital microscopy color cameras commercially available from various manufactures including but not limited to e.g., Dino-Eye, Dino-Lite, Jenoptik ProgRes, KoPa, Leica, Motic, Olympus, Omano, OptixCam, PixeILINK, Zeiss, etc.

Cell cytometers further include data acquisition, analysis and recording means, such as a computer, wherein one or more data channels record data from one or more image capture devices for each cell or most of the cells of the imaging vessel. The purpose of the analysis system is to classify and count cells wherein each cell presents itself as a set of digitized parameter values and to accumulate data for the sample as a whole. In some cases, cell cytometers record images of each cell and may be connected to a user interface where such images may be reviewed by a user of the device.

Cell cytometer based methods for detecting cells expressing a particular polypeptide may include contacting the cells of a sample with a fluorescent labeled specific binding member and detecting fluorescently labeled cells by imaging using the cell cytometer. In some instances, the fluorescence of each labeled cell may be cytometrically quantified to identify the per cell expression level of a particular marker.

Any cell cytometer that is capable of obtaining fluorescence data, e.g., as described above, may be employed. Useful cell cytometers include those utilizing various different means of automated cell cytometric imaging to analyze all or most of the cells of a sample. Non-limiting examples of cell cytometer systems of interest are those available from commercial suppliers including but not limited to, e.g., Nexcelom Bioscience LLC (Lawrence, Mass.), Molecular Devices, LLC (Sunnyvale, Calif.), Thorlabs Inc. (Newton, N.J.), TTP Labtech Ltd. (United Kingdom), and the like.

As summarized above, one of the parameters that may be cytometrically assayed is DNA content (also referred to as DNA index) of the analyzed cells, e.g., through the use of a DNA staining reagent applied to the cells prior to the assaying. For example, in some instances the DNA content of cells may be qualitatively or quantitatively assessed, including but not limited to e.g., where the per cell DNA content is quantitated based on the DNA staining reagent. Such procedures may serve in determining the relative and/or absolute DNA content of an assessed cell. As such, DNA content may be employed as a marker, including a marker of a marker panel, in the assays as described herein.

Various methods may be employed for determining the DNA content of a cell, e.g., the per cell DNA content. In some instances, a DNA labeling reagent (e.g., a nucleic acid dye or stain that contains intrinsic fluorescence) may be employed to label the DNA of the cell and the amount of DNA may be quantified based on measuring the intensity of the label. In one embodiment, e.g., regardless of the type of cytometry employed (e.g., flow cytometry, cell cytometry, etc.), the fluorescent intensity of cells labeled with a DNA labeling reagent may analyzed on the cytometer and plotted on a histogram. From the histogram the relative amount of DNA content may be determined for each cell.

In some instances, DNA content of a cell may be expressed as a measure of aneuploidy of the cell. Any convenient method of measuring aneuploidy cytometrically may be employed in the subject methods. In some instances, a cell may be identified as aneuploid based on the measured DNA content of the cell where an aneuploid cell will generally have an abnormally high level of DNA content representing duplication of all or a portion of the cell's genome. Similar methods to those described above for assessing DNA content in regards DNA content may be employed for detecting aneuploidy. In some instances, relative DNA content greater than or equal to a threshold DNA content value for a normal cell may indicate that the cell is aneuploid where the threshold may be greater than or equal to (≥) 1.05 times the DNA content of a normal cell including but not limited to, e.g., 1.06 times, 1.07 times, 1.08 times, 1.09 times, 1.10 times, 1.11 times, 1.12 times and 1.13 times the DNA content of a normal cell.

Ploidy assessments (e.g., assessing the ploidy of a cell, including e.g., whether a cell is aneuploid, diploid, etc.) may be employed for various purposes. For example, in some instances, a ploidy assessment may be employed to determine whether cells of a population are aneuploid or diploid, including e.g., to determine whether a neoplastic cell is aneuploid or diploid, whether an immune cell is aneuploid or diploid, or the like. In some instances, a ploidy assessment may inform other characteristics of the sample and/or the subject, e.g., by a relationship between the ploidy status of a detected cell and other cell types that may be present in the subject.

As summarized above, one of the parameters that may be cytometrically assayed is the cell cycle of the analyzed cells, e.g., through the use of a cell cycle analysis, including where a reagent to assess cell cycle is applied to the cells prior to the assaying. For example, in some instances the cell cycle of cells may be qualitatively or quantitatively assessed, including but not limited to e.g., where the cell cycle position of a cell (or a plurality of cells) is determined or quantitated based on a reagent added to assay cell cycle. As such, cell cycle may be employed as a marker, including a marker of a marker panel, in the assays as described herein.

In some instances, cell cycle data may include but is not limited to determining whether a cell is in G₁ phase of the cell cycle, the count or percent of cells that are in G₁ phase of the cell cycle, whether a cell is in G₂ phase of the cell cycle, the count or percent of cells of the that are in G₂ phase of the cell cycle, whether a cell is in S phase of the cell cycle, the count or percent of cells that are in S phase of the cell cycle, whether a cell is in M phase of the cell cycle, the count or percent of cells that are in M phase of the cell cycle, whether a cell is in G₀ phase of the cell cycle, the count or percent of cells that are in G₀ phase of the cell cycle, and combinations thereof. Useful combinations of cell cycle determinations and/or counts or percentages of cells of the sample that are in particular phases of the cell cycle include but are not limited to e.g., “post G₁” which includes e.g., a combination of S, G₂ and M. In some instances, such analyses may be based on labeling of the cells with a DNA labeling reagent, such as a fluorescent DNA stain or labeling reagent.

Markers employed in assessing cell cycle may, in some instances, include but are not limited to e.g., cell cycle and/or proliferation markers (e.g., Ki-67, phosphohistone H3, proliferating cell nuclear antigen (PCNA), cyclins, cyclin-dependent kinases, retinoblastoma, etc.), and the like. Such markers may be detected through the use of a binding member that is specific for the marker, including where the marker allows for assaying the expression of the marker at the protein or mRNA level.

In some instances, the methods of the instant disclosure may include determining whether a subject cell is or is not an immune cell. In some instances, the methods of the instant disclosure may include determining whether a subject cell is or is not a particular type of immune cell (e.g., a lymphocyte or progenitor thereof, a myeloid progenitor, a granulocyte, a thymocyte, a myeloid precursor, a T cell, a B cell, a NK cell, a T helper cell, a Treg, a neutrophil, an eosinophil, a basophil, a mast cell, a monocyte, a dendritic cell, a macrophage, a memory cell, a plasma cell, a hematopoietic stem cell, etc.). Various methods may be employed for determining whether a subject cell is or is not an immune cell or determining whether or not a cell is a particular type of immune cell including e.g., through detecting the presence or absence of one or more markers (including immune cell markers and non-immune cell markers) e.g., through contacting the cell with a labeled specific binding member specific for the marker. In some instances, immune cell type determinations may include assessing whether a cell expresses a particular marker, or group of markers, at a high or low level. Such levels may be relative, e.g., based on the level of marker expression in other cells of the analysis, or absolute, e.g., based on a predetermined threshold expression level.

Useful markers may include but are not limited to e.g., one or more of the immune cell-type markers described herein. In some instances, the expression, e.g., the presence or absence of expression, of one or more markers (including immune cell markers and non-immune cell markers), e.g., apart from, in addition to, or in place of one or more of the immune cell-type markers described herein, may be employed. Non-limiting examples of useful markers include, alone or in combination, CD45, CD15, CD3, CD4, CD8, CD25, CD19, CD20, CD11c, CD123, CD56, CD34, CD38, CD14, CD39, CD33, CD66b, CD41, CD61, CD62, CD235a, CD146, CD326, FoxP3, and the like.

In some instances, the size of a detected cell population may be expressed in relative terms. For example, the size of the population may be expressed as a percentage of all the cells in the sample, a percentage of all the cells analyzed, a percentage of all of the cells of a particular type within the sample, a percentage of all of the cells of a particular type that were analyzed, etc. In some instances, the size of the detected population may exceed 0.01% or more of the neoplastic cells in the cell suspension sample, including but not limited to e.g., 0.1% or more, 1% or more, 10% or more, etc.

### Reagents

As summarized above, the instant methods include the detection of a cancer-related cell type and/or a population thereof expressing one or more markers and thus include various reagents useful in practicing the methods. For example, the instant methods generally include detecting a cancer-related cell type, or population thereof, expressing or having a lack of expression of one or more markers by contacting the cell suspension with a labeled specific binding member reagent in order to allow cytometric assays to be performed.

Specific binding agents of interest include but are not limited to antibody binding agents, proteins, peptides, haptens, nucleic acids, etc. The term “antibody binding agent” as used herein includes polyclonal or monoclonal antibodies or fragments that are sufficient to bind to an analyte of interest. The antibody fragments can be, for example, monomeric Fab fragments, monomeric Fab′ fragments, or dimeric F(ab)′₂ fragments. Also within the scope of the term “antibody binding agent” are molecules produced by antibody engineering, such as single-chain antibody molecules (scFv) or humanized or chimeric antibodies produced from monoclonal antibodies by replacement of the constant regions of the heavy and light chains to produce chimeric antibodies or replacement of both the constant regions and the framework portions of the variable regions to produce humanized antibodies.

Useful nucleic acid binding members specifically bind to the marker, including protein markers and nucleic acid markers. For example, useful nucleic acid binding members may include but are not limited to e.g., nucleic acid binding members that specifically bind a protein marker, such an aptamer, and nucleic acid probes that specifically bind a nucleic acid marker, such as a nucleic acid probe that specifically hybridizes with a target nucleic acid biomarker. Specific binding of a nucleic acid binding member to its corresponding nucleic acid marker may involve hybridization of the nucleic acid binding member and the nucleic acid marker under stringent hybridization conditions. Nucleic acid binding members may, as described in more detail below, be directly or indirectly labeled. Indirect labeling of nucleic acid binding members may or may not include signal amplification, such as but not limited to e.g., branched nucleic acid (e.g., bDNA) signal amplification, tyramide signal amplification, and the like.

Specific binding members described herein may be directly or indirectly detectably labeled (i.e., have an attached detectable label, be bound by a detectable label, etc.). Therefore, in addition to a specific binding domain that specifically binds to the biomarker of interest, the specific binding agent may further include or may be bound by or attached to a detectable label. Of interest as detectable labels are fluorescent dyes. Fluorescent dyes (fluorophores) can be selected from any of the many dyes suitable for use in imaging applications (e.g., fluorescent microscopy) and cytometry applications. A large number of dyes are commercially available from a variety of sources, such as, for example, Molecular Probes (a division of Thermo Fisher Scientific, Waltham, Mass. USA) and Exciton (Dayton, Ohio). Examples of fluorophores of interest include, but are not limited to, 4-acetamido-4′-isothiocyanatostilbene-2,2′disulfonic acid; acridine and derivatives such as acridine, acridine orange, acridine yellow, acridine red, and acridine isothiocyanate; 5-(2′-aminoethyl)aminonaphthalene-1-sulfonic acid (EDANS); 4-amino-N-[3-vinylsulfonyl)phenyl]naphthalimide-3,5 disulfonate (Lucifer Yellow VS); N-(4-anilino-1-naphthyl)maleimide; anthranilamide; Brilliant Yellow; coumarin and derivatives such as coumarin, 7-amino-4-methylcoumarin (AMC, Coumarin 120), 7-amino-4-trifluoromethylcouluarin (Coumarin 151); cyanine and derivatives such as cyanosine, Cy3, Cy5, Cy5.5, and Cy7; 4′,6-diaminidino-2-phenylindole (DAPI); 5′, 5″-dibromopyrogallol-sulfonephthalein (Bromopyrogallol Red); 7-diethylamino-3-(4′-isothiocyanatophenyl)-4-methylcoumarin; diethylaminocoumarin; diethylenetriamine pentaacetate; 4,4′-diisothiocyanatodihydro-stilbene-2,2′-disulfonic acid; 4,4′-diisothiocyanatostilbene-2,2′-disulfonic acid; 5-[dimethylamino]naphthalene-1-sulfonyl chloride (DNS, dansyl chloride); 4-(4′-dimethylaminophenylazo)benzoic acid (DABCYL); 4-dimethylaminophenylazophenyl-4′-isothiocyanate (DABITC); eosin and derivatives such as eosin and eosin isothiocyanate; erythrosin and derivatives such as erythrosin B and erythrosin isothiocyanate; ethidium; fluorescein and derivatives such as 5-carboxyfluorescein (FAM), 5-(4,6-dichlorotriazin-2-yl)aminofluorescein (DTAF), 2′7′-dimethoxy-4′5′-dichloro-6-carboxyfluorescein (JOE), fluorescein isothiocyanate (FITC), fluorescein chlorotriazinyl, naphthofluorescein, and QFITC (XRITC); fluorescamine; IR144; IR1446; Green Fluorescent Protein (GFP); Reef Coral Fluorescent Protein (RCFP); Lissamine™; Lissamine rhodamine, Lucifer yellow; Malachite Green isothiocyanate; 4-methylumbelliferone; ortho cresolphthalein; nitrotyrosine; pararosaniline; Nile Red; Oregon Green; Phenol Red; B-phycoerythrin; o-phthaldialdehyde; pyrene and derivatives such as pyrene, pyrene butyrate and succinimidyl 1-pyrene butyrate; Reactive Red 4 (Cibacron™ Brilliant Red 3B-A); rhodamine and derivatives such as 6-carboxy-X-rhodamine (ROX), 6-carboxyrhodamine (R6G), 4,7-dichlororhodamine lissamine, rhodamine B sulfonyl chloride, rhodamine (Rhod), rhodamine B, rhodamine 123, rhodamine X isothiocyanate, sulforhodamine B, sulforhodamine 101, sulfonyl chloride derivative of sulforhodamine 101 (Texas Red), N,N,N′,N′-tetramethyl-6-carboxyrhodamine (TAMRA), tetramethyl rhodamine, and tetramethyl rhodamine isothiocyanate (TRITC); riboflavin; rosolic acid and terbium chelate derivatives; xanthene; or combinations thereof. Other fluorophores or combinations thereof known to those skilled in the art may also be used, for example those available from Molecular Probes (a division of Thermo Fisher Scientific, Waltham, Mass. USA) and Exciton (Dayton, Ohio).

The methods of the present disclosure may include the use of a labeled binding member specific for a marker, such as e.g., an immune checkpoint marker, an immune cell-type marker, an immune activation marker, an adhesion-related marker, a mesenchymal cell marker, an epithelial cell marker, etc., to label cells of the cell suspension thus generating a labeled cell suspension that may be cytometrically assayed. In some instances, depending on the context, a labeled binding member specific for one of the following markers: PD-1, TIM-3, LAG-3, CTLA-4, CD3, CD4, CD8, CD16, CD19, CD56, HLA-DR, E cadherin, CD44, CD103, Pan CK, or vimentin, may be employed. In some instances, a combination of labeled binding members each specific for one of the following markers: PD-1, TIM-3, LAG-3, CTLA-4, CD3, CD4, CD8, CD16, CD19, CD56, HLA-DR, E cadherin, CD44, CD103, Pan CK, or vimentin, may be employed. In some instances, a combination of labeled binding members each specific for a particular marker may include labeled binding members for one or more of the following markers: CD4, CD25, CD103, CD127, and/or CCR5. In some instances, a combination of labeled binding members each specific for one of the following markers: CD8, CD4, CD45, CCR5, CD39, CD103, CD3, CD56, LAG-3, CD14, Pan CK, PD-1, FoxP3, TIM-3, CD19, CTLA-4, PD-L1, CD16, or HLA-DR, may be employed. In some instances, a labeled binding member specific for a marker, e.g., one of the markers described herein, may specifically bind the marker protein expressed on the surface of a cell, e.g., a human cell.

PD-1 (CD279) is an immune checkpoint marker expressed on immune cells and, when functioning as an immune checkpoint, PD-1 down regulates the immune response, thereby reducing autoimmunity and promoting self-tolerance. PD-1 is expressed on immune cell types, including most mature T cells located in peripheral tissue. During an immune response PD-1 has two ligands: PD ligand 1 (PDL-1), also known as B7-H1 and CD274, and PD ligand 2 (PDL-2), also known as B7-DC and CD273. Examples of antibodies against PD-1 are described in U.S. Pat. Nos. 7,488,802; 8,217,149; and (Topalian, S. L., Hodi, F. S., Brahmer, J. R., Gettinger, S. N., Smith, D. C., McDermott, D. F., . . . Sznol, M. (2012). Safety, Activity, and Immune Correlates of Anti-PD-1 Antibody in Cancer. The New England Journal of Medicine, 366(26), 2443-2454). Suitable antibodies targeting PD-1 include, but are not limited to, the anti-PD1 antibody [Clone NAT105] (ab52587) from Abcam Inc. In humans, the PD-1 protein (RefSeq ID NP_005009.2 corresponding to NCBI reference sequence NM_005018.2; Uniprot ID 015116) is encoded by the PDCD1 gene (UniGene ID 155258). TIM-3 (also known as T-cell immunoglobulin mucin receptor 3, T-cell immunoglobulin and mucin domain-containing protein 3, Hepatitis A virus cellular receptor 2, HAVcr-2, and the like) is an immune checkpoint marker expressed by immune cells, including CD4+ and CD8+ T cells, and is expressed in many other tissues. Upon binding to galectin, TIM-3 may initiate a number of signaling pathways involved in the negative regulation of the immune response such as: reducing T cell function, increasing CD4+ T cell death, promoting the development of myeloid derived suppressor cells, and the like. Examples of antibodies against TIM-3 are described in U.S. Patent Application No. 2015/0218274; U.S. Pat. No. 8,552,156; (Sakuishi, Kaori, et al. “Targeting Tim-3 and PD-1 Pathways to Reverse T Cell Exhaustion and Restore Anti-Tumor Immunity.” The Journal of Experimental Medicine, vol. 207, no. 10, 2010, pp. 2187-2194.); (Du, W., Yang, M., Turner, A., Xu, C., Ferris, R. L., Huang, J., . . . Lu, B. (2017). TIM-3 as a Target for Cancer Immunotherapy and Mechanisms of Action. International Journal of Molecular Sciences, 18(3), 645.); and (Ngiow S. F., B. von Scheidt, H. Akiba, H. Yagita, M. W. Teng, M. J. Smyth. 2011. Anti-TIM3 antibody promotes T cell IFN-γ-mediated antitumor immunity and suppresses established tumors. Cancer Res. 71: 3540-3551.) Suitable antibodies targeting Tim-3 include, but are not limited to, the PE anti-TIM-3 monoclonal antibody (Clone F38-2E2) from BioLegend. In humans, the TIM-3 protein (RefSeq ID NP_116171.3 corresponding to NCBI reference sequence NM_032782.4; Uniprot ID Q8TDQ0) is encoded by the Hepatitis A virus cellular receptor 2 (HAVCR2) gene (UniGene ID 3322707).

LAG-3 (CD223) is an immune checkpoint marker and a cell surface receptor expressed on immune cells. Activated T cells (CD4+ and CD8+) and NK cells express LAG-3 and upregulate its expression in response to pro-inflammatory cytokines such as interferon gamma (IFN-γ). LAG-3 binds MHC class II thereby preventing MHC class II binding with the TCR and thus inhibiting T cell stimulation. The binding of LAG-3 to MHC class II on dendritic cells (DCs) may result in activation of signaling pathways that upregulate TNF-α and IL-12. Examples of antibodies against LAG-3 are described in U.S. Patent Application No. 20110150892; U.S. Patent Application No. 20150259420; (Goldberg. M. V., & Drake, C. G. (2011). LAG-3 in Cancer Immunotherapy. Current Topics in Microbiology and Immunology, 344, 269-278.); and (Andrews, L. P., Marciscano, A. E., Drake, C. G., & Vignali, D. A. A. (2017). LAGS (CD223) as a Cancer Immunotherapy Target. Immunological Reviews, 276(1), 80-96.). Suitable antibodies targeting LAG-3 include, but are not limited to, the anti-LAG 3 antibody [Clone 11 E3] (ab40465) from Abeam Inc. and the anti-LAG3 monoclonal antibody (BMS-986016) from Bristol Meyers Squibb Co. In humans, the LAG-3 protein (RefSeq ID NP 002277.4 corresponding to NCBI reference sequence NM_002286.5; Uniprot ID P18627) is encoded by the Lymphocyte-activation gene 3 (LAG3) gene (UniGene ID 221341).

CTLA-4 (CD152) is an immune checkpoint marker and a receptor expressed on immune cells, including on T cells, such as, e.g., CD4+, CD8+ and Tregs. CTLA-4 is a single pass type 1 transmembrane protein belonging to the immunoglobulin superfamily containing a single Ig-v-like domain in the extracellular region. Once bound to B7-1 (CD80) and B7-2 (CD86) proteins, CTLA-4 negatively regulates T cell activation. Examples of antibodies against CTLA-4 are described in U.S. Pat. Nos. 6,682,736; 7,605,238; (Callahan. M. K., Wolchok, J. D., & Allison, J. P. (2010). Anti-CTLA-4 Antibody Therapy: Immune Monitoring During Clinical Development of a Novel Immunotherapy. Seminars in Oncology, 37(5), 473-484.); and (Blank, C. U., and A. Enk. “Therapeutic Use of Anti-CTLA-4 Antibodies.” International Immunology, vol. 27, no. 1, 2014, pp. 3-10.). Suitable antibodies targeting CTLA-4 include, but are not limited to, the anti-CTLA4 antibody [Clone EPR1476] (ab134090) from Abcam Inc. and the anti-CTLA-4 antibody (YERVOY®) from Bristol-Meyers Squibb Co. In humans, the CTLA-4 protein (Uniprot ID P16410) may be present in multiple isoforms (e.g., RefSeq ID NP_005205.2 and NP_001032720.1 corresponding to NCBI reference sequences NM_005214.4 and NM 001037631.2) which are encoded by the Cytotoxic T-lymphocyte-associated protein 4 (CTLA4) gene (UniGene ID 168967).

CD3 (cluster of differentiation 3) is an immune cell-type marker and a T cell co-receptor that is involved in the activation of immune cell responses, including those of cytotoxic T cells (CD8+ naive T cells) and T helper cells (CD4+ naive T cells). CD3 is a protein complex composed of four distinct chains (a CD3γ chain, a CD3δ chain, and two CD3ε chains in mammals). The CD3 chains associate with the T-cell receptor (TCR) and the ζ-chain (zeta-chain) to generate an activation signal in T lymphocytes. The TCR, ζ-chain, and CD3 molecules together constitute the TCR complex. Suitable antibodies targeting CD3 include, but are not limited to, the anti-CD3 antibody (ab5690) from Abcam Inc. and the anti-humanCD3 antibody (Clone OKT3) from BioLegend. Antibodies to CD3 may be directed to any of the individual CD3 chains (i.e., CD3γ chain, CD3δ chain, or CD3ε chain. In humans, the T-cell surface glycoprotein CD3 epsilon chain (CD3E) (RefSeq ID NP_000724.1 corresponding to NCBI reference sequence NM_000733.3; Uniprot ID P07766) is encoded by the CD3e molecule, epsilon (CD3-TCR complex) (CD3E) gene (UniGene ID 131335). In humans, the T-cell surface glycoprotein 003 gamma chain (CD3G) (RefSeq ID NP_000064.1 corresponding to NCBI reference sequence NM_000073.2; Uniprot ID P09693) is encoded by the CD3g molecule, gamma (CD3-TCR complex) (CD3G) gene (UniGene ID 131196). In humans, the T-cell surface glycoprotein CD3 delta chain (CD3D) may be present in multiple isoforms (e.g., RefSeq ID NP 000723,1 and NP 038515.3 corresponding to NCBI reference sequences NM 000732.4 and NM 001040651.1 Uniprot ID P04234) encoded by the CD3d molecule, delta (CD3-TCR complex) (CD3D) gene (UniGene ID 719207).

CD4 (cluster of differentiation 4) is an immune cell-type marker and a member of the immunoglobulin superfamily, having four immunoglobulin domains (D1 to D4) that are exposed on the extracellular surface of the cell. CD4 interacts with the β2-domain of MHC class II molecules through its D1 domain. CD4 is a glycoprotein found on the surface of immune cells such as T helper cells, monocytes, macrophages, and dendritic cells. Suitable antibodies targeting CD4 include, but are not limited to, the anti-CD4 antibody [Clone EPR6855] (ab133616) from Abcam Inc. and the APC anti-human CD4 antibody (Clone RPA-T4) from BioLegend. In humans, the 004 protein (RefSeq ID NP_000607.1 corresponding to NCBI reference sequence NM_000616.4; Uniprot ID P01730) is encoded by the CD4 molecule (004) gene (UniGene ID 2138553).

CD8 (cluster of differentiation 8) is an immune cell-type marker and a transmembrane glycoprotein that serves as a co-receptor for the T cell receptor (TCR). CD8 binds to a major histocompatibility complex (MHC) molecule and is specific for the class I MHC protein. The CD8 co-receptor is predominantly expressed on the surface of cytotoxic T cells and may serve as a marker for this population. CD8 is expressed on a variety of immune cells including e.g., natural killer cells, cortical thymocytes, and dendritic cells. Suitable antibodies targeting CD8 include, but are not limited to, the anti-CD8 antibody (ab4055) from Abcam Inc. and the PE anti-human CD8 antibody (Clone SK1) from BioLegend. The 008 protein is composed of a CD8a and CD8β chain. Antibodies to CD8 may be directed to either the alpha or beta chain. In humans, the T-cell surface glycoprotein 008 alpha chain (CD8A) may be present in multiple isoforms (e.g., RefSeq ID NP_001139345.1, NP_001759.3 and NP 741969.1 corresponding to NCBI reference sequences NM_001145873.1, NM_001768.6 and NM_171827.3; Uniprot ID P01732) encoded by the 008a molecule (008A) gene (UniGene ID 140849). In humans, the T-cell surface glycoprotein CD8 beta chain (CD8B) may be present in multiple isoforms (e.g., RefSeq ID NP_001171571.1, NP_004922.1 NP_742099.1, NP 742100.1, and NP 757362.1 corresponding to NCBI reference sequence NM_004931.4, NM_172101.3, NM_172102.3 and NM_172213.3; Uniprot ID P10966) encoded by the CD8b molecule (CD8B) gene (UniGene ID 219625).

CD14 (cluster of differentiation 14, also known as monocyte differentiation antigen CD14 and myeloid cell-specific leucine-rich glycoprotein) exists in two forms: a cell surface expressed form anchored to the membrane by a glycosylphosphatidylinositol tail (mCD14) and a soluble form (sCD14). The surfaced expressed form of CD14 serves as an immune cell-type marker that can be found on the surface of immune cells, including macrophages, neutrophils and dendritic cells. CD14⁺ monocytes can differentiate into a host of different cells. Structural studies (see e.g., PDB: 4GLP, indicate that 0014 includes a monomeric, bent solenoid structure containing a hydrophobic amino-terminal pocket. Suitable antibodies targeting CD14 include, but are not limited to, the FITC anti-human CD14 Antibody [clone HCD14] from BioLegend; Anti-CD14 Antibody [clone 2D-15C] from Sigma-Aldrich, Inc.; Anti-CD14-FITC Antibody [clone TUK4] from Sigma-Aldrich, Inc.; CD14 Antibody (5A3B11 B5) sc-58951 from Santa Cruz Biotechnology, Inc.; Anti-CD14 antibody [4B4F12] (ab182032) from Abcam Inc.; and the like. The CD14 protein is a surface antigen that is preferentially expressed on monocytes/macrophages. It cooperates with other proteins to mediate the innate immune response to bacterial lipopolysaccharide. Alternative splicing results in multiple transcript variants encoding the same protein. In humans, the CD14 protein may be present in multiple isoforms, including one major isoform and three potential isoforms (e.g., RefSeq IDs NP_000582.1, NP_001035110.1, NP_001167575.1, and NP_001167576.1, corresponding to NCBI reference sequences NM_000591.3, NM_001040021.2, NM_001174104.1, and NM_001174105.1 respectively; Uniprot ID P08571) encoded by the CD14 gene (UniGene ID Hs.163867).

CD16 (cluster of differentiation 16, also known as FcγRIII) is an immune cell-type marker found on the surface of immune cells including e.g., natural killer cells, neutrophil polymorphonuclear leukocytes, monocytes and macrophages. CD16 has a conserved immunoglobulin-like (Ig-like) structure that includes two immunoglobulin-like domains CD16 is involved in antibody-dependent cellular cytotoxicity (ADCC) and can be used to isolate populations of specific immune cells through fluorescent-activated cell sorting (FACS) or magnetic-activated cell sorting. Suitable antibodies targeting CD16 include, but are not limited to, the anti-human CD16 antibody (Clone 3G₈) from BioLegend and the anti-CD16 antibody [Clone SP175] (ab183354) from Abcam Inc. CD16 has been identified as Fc receptors FcγRIIIa (CD16a) and FcγRIIIb (CD16b) and antibodies to CD16 may be directed to either form. In humans, the Low affinity immunoglobulin gamma Fc region receptor III-A (FCGR3A, CD16s) protein may be present in multiple isoforms (e.g., RefSeq ID NP_000560.6, NP_001121064.2, NP_001121065.1 NP_001121067.1, NP_001121068.1, and NP_001316049.1 corresponding to NCBI reference sequence NM_0005697, NM_001127592.2, NM_001127593.1, NM_001127595.1, NM_001127596.1, and NM_001329120.1; Uniprot ID P08637) encoded by the Fe fragment of IgG, low affinity IIIa, receptor (CD16a) (FCGR3A) gene (UniGene ID 199972). In humans, the Low affinity immunoglobulin gamma Fc region receptor III-B (FCGR3B, CD16b) protein may be present in multiple isoforms (e.g., RefSeq ID NP_000561.3, NP_001231682.1, NP_001257964.1, NP_001257965.1. and NP_001257966.1 corresponding to NCBI reference sequences NM_000570.4, NM_001244753.1, NM_001271035.1. NM_001271036.1, and NM_001271037.1; Uniprot ID 075015) encoded by a Fc fragment of IgG, low affinity IIIb, receptor (CD16b) (FCGR3B) gene (e.g., UniGene ID 2845101 or 5800047).

CD19 (cluster of differentiation 19; also known as B-lymphocyte antigen CD19, B-Lymphocyte Surface Antigen B4, T-Cell Surface Antigen Leu-12, CVID3 etc.) is an immune cell-type marker and is a transmembrane protein that in humans is encoded by the gene CD19 located on the short arm of chromosome 16. CD19 is a 95 kd Type I transmembrane glycoprotein in the immunoglobulin superfamily (IgSF) with two extracellular C2-set Ig-like domains and a cytoplasmic tail that is highly conserved among mammalian species. CD19 is widely expressed during all phases of B cell development until terminal differentiation into plasma cells. Suitable antibodies targeting CD19 include, but are not limited to, the anti-CD19 antibody [Clone 6D5] (ab25232) from Abcam Inc. and the FITC anti-human CD19 antibody (Clone HIB19) from BioLegend. In humans, the CD19 protein may be present in multiple isoforms (e.g., RefSeq ID NP_001171569.1 and NP_301761.3 corresponding to NCBI reference sequences NM_301178098.1 and NM_001770.5; Uniprot ID P15391) encoded by the CD19 molecule (CD19) gene (UniGene ID 2559235).

CD39 (Cluster of Differentiation 39, also known as the protein expressed from the Ectonucleoside triphosphate diphosphohydrolase-1 (ENTPD1) gene and NTPDase1) is an immune cell-type marker and is a typical cell surface-located enzyme with an extracellularly facing catalytic site. Suitable antibodies targeting CD39 include, but are not limited to, the CD39 (human) monoclonal antibody (AC2.5) from Enzo Life Sciences, Inc.; the FITC and PE/Cy7 CD39 antibodies from BioLegend; the APC and FITC cd39 antibodies from Miltenyi Biotec; the CD39/ENTPD1 Alexa Fluor 350-conjugated Antibody from R&D Systems; and the Ike. The CD39 protein is a plasma membrane protein that hydrolyzes extracellular ATP and ADP to AMP and inhibition of its activity may confer anticancer benefits. Several transcript variants encoding different isoforms have been found. In humans, the CD39 protein may be present in multiple isoforms (e.g., RefSeq IDs NP_001240.1, NP_001308914.1, NP_001308915.1, NP_001308916.1, NP_001308917.1, corresponding to NCBI reference sequences NM_001249.3, NM_001321985.1, NM_001321986.1, NM_001321987.1, and NM_001321988.1_ respectively; Uniprot ID 075356) encoded by the ENTPD1 gene (UniGene ID Hs.655070, Hs.656955, Hs.720540).

CCR5 (C-C chemokine receptor type 5, also known as CD195) is an immune cell-type marker and is a protein on the surface of white blood cells that is involved in the immune system as it acts as a receptor for chemokines. Suitable antibodies targeting CCR5 include, but are not limited to, the Anti-CD195 (CCR5) (Human) mAb-FITC from MBL International; the CCR5 Alexa Fluor 350-conjugated Antibody from R&D Systems; the APC Recombinant Anti CD195 (CCR5) Antibody from Miltenyi Biotec; the Alexa Fluor 647 and 700 CD195 (CCR5) Antibody from BioLegend; the Anti-CCR5 antibody [HEK/1/85a] (FITC) from Abcam; and the Ike. The CCR5 protein is a member of the beta chemokine receptor family, which is predicted to be a seven transmembrane protein similar to G protein-coupled receptors. This protein is expressed by T cells and macrophages, and is known to be an important co-receptor for macrophage-tropic virus, including HIV, to enter host cells. Defective alleles of this gene have been associated with the HIV infection resistance. In humans, the CCR5 protein may be present in multiple isoforms (e.g., RefSeq ID NP_000570.1 corresponding to NCBI reference sequence NM_000579.3 and NP_001093638.1 corresponding to NCBI reference sequence NM_001100168.1; Uniprot ID P51681) encoded by the CCR5 gene (UniGene ID Hs.450802).

CD56 (also known as neural cell adhesion molecule (NCAM)) is an immune cell-type marker and is a homophilic binding glycoprotein expressed on the surface of various cells including natural killer (NK) cells. CD56 has been detected on other lymphoid cells, including gamma delta (γδ) T cells and activated CD8+ T cells, as well as on dendritic cells. Suitable antibodies targeting CD56 include, but are not limited to, the PE anti-human CD56 antibody (Clone HCD56) from BioLegend and the anti-CD56 antibody (Clone 123C3) from Bio-Rad. In humans, the CD56 protein may be present in multiple isoforms (e.g., RefSeq ID NP_000606.3, NP_001070150.1, NP_001229537.1, and NP_851996.2 corresponding to NCBI reference sequences NM_000615.6, NM_001076682.3, NM_001242608.1, and NM_181351.4; Uniprot ID P13591) encoded by a Neural cell adhesion molecule 1 (NCAM1) gene (e.g., UniGene ID 719037, 3323442 and 5796848).

FoxP3 (forkhead box P3, also known as scurfin) is an immune cell-type marker and is member of the FOX protein family and functions as a master regulator of the regulatory pathway in the development and function of regulatory T cells. Suitable antibodies targeting FoxP3 include, but are not limited to, the FOXP3 FITC conjugated Antibody (Mouse mAb) from Abeomics the AFC Anti FoxP3 Antibody from Miltenyi Biotec; the FoxP3 Antibody (3G₃) [Alexa Fluor 405] from Novus Biologicals; the Anti-FOXP3-Alexa Fluor® 647, clone 3G₃ from MilliporeSigma; and the like. Antibodies to In humans, the FoxP3 protein may be present in multiple isoforms (e.g., RefSeq ID NP_ NP_001107849.1 corresponding to NCBI reference sequence NM_001114377.1 and NP_054728.2 corresponding to NCBI reference sequences NM_014009.3; Uniprot ID Q9BZS1) encoded by the FoxP3 gene (UniGene ID Hs.247700).

HLA-DR (Human Leukocyte Antigen-antigen D Related) is an immune activation marker and is MHC class II molecule, transmembrane glycoprotein composed of an alpha chain (36 kDa) and a beta chain (27 kDa). It is expressed primarily on antigen presenting cells such as B lymphocytes, monocytes, macrophages, thymic epithelial cells and activated T lymphocytes. HLA-DR is a cell surface receptor encoded by the human leukocyte antigen complex on chromosome 6 region 6p21.31. Suitable antibodies targeting HLA-DR include, but are not limited to, the HLA-DR/DQ Monoclonal Antibody (49.1), FITC from ThermoFisher Scientific, the PE anti-human HLA-DR Antibody anti-HLA-DR L243 from BioLegend, the HLA-DR Monoclonal Antibody (L243), PE, eBioscience™ from ThermoFisher Scientific, the Anti-HLA-DR antibody [TAL 1B5] (ab20181) from Abcam Inc., and the like. In humans, the HLA class II histocompatibility antigen, DR alpha chain (RefSeq NP_061984.2 corresponding to NCBI reference sequences NM_019111.4; Uniprot ID P01903) is encoded by the Major histocompatibility complex, class II, DR alpha (HLA-DRA) gene (UniGene ID 910795). In humans, the HLA class II histocompatibility antigen, DR beta chain may be present in multiple isoforms (e.g., RefSeq NP_301230894.1 and NP_002116.2 corresponding to NCBI reference sequences NM_001243965.1 and NM_002125.3; Uniprot IDs P04229, Q30154, P01911, Q9GIY3) encoded by a HLA-DRB (UniGene IDs 5800377, 2976816 and 1370636).

E cadherin (also known as CD324 (cluster of differentiation 324), CAM 120/80, epithelial cadherin, uvomorulin, etc.) is an adhesion-related marker encoded by the CDH1 gene. E cadherin is a classical member of the cadherin superfamily. E-cadherin levels change in EMT (epithelial-mesenchymal transition) and MET (mesenchymal-epithelial transition). Suitable antibodies targeting E cadherin include, but are not limited to, the FITC anti-human CD324 E-Cadherin Antibody 67A4 from BioLegend, the PE Mouse anti-E-Cadherin (Clone 36/E-Cadherin) from BD Biosciences, the Human E-Cadherin PE-conjugated Antibody FAB18381P from R&D Systems, and the like. In humans, the E cadherin protein may be present in multiple isoforms (e.g., RefSeq NP_001304113.1, NP_001304114.1, NP_001304115.1 and NP_004351.1 corresponding to NCBI reference sequences NM_001317184.1, NM_001317185.1, NM_001317186.1 and NM_004360.4; Uniprot ID P12830) encoded by the Cadherin 1, type 1, E-cadherin (epithelial) (CDH1) gene (UniGene ID 676245).

CD44 (also known as HCAM (homing cell adhesion molecule), Pgp-1 (phagocytic glycoprotein-1), Hermes antigen, lymphocyte homing receptor, ECM-III, HUTCH-1, etc.) is an adhesion-related marker and a cell-surface glycoprotein involved in cell-cell interactions, cell adhesion and migration encoded by the CD44 gene on Chromosome 11. CD44 is expressed in a large number of mammalian cell types. Suitable antibodies targeting CD44 include, but are not limited to, the Anti-CD44 antibody [F10-44-2] (ab6124) from Abcam Inc., the FITC anti-human CD44 Antibody BJ18 from BioLegend, the Human/Mouse CD44 Antibody MAB6127 from R&D Systems, and the like. In humans, the CD44 protein may be present in multiple isoforms (e.g., RefSeq NP_000601.3, NP_001001389.1, NP_001001390.1, NP_001001391.1, NP_001001392.1, NP_001189484.1, NP_001189485.1 and NP_001189486.1, corresponding to NCBI reference sequences NM_000610.3, NM_001001389.1, NM_001001390.1, NM_001001391.1, NM_001001392.1, NM_001202555.1, NM_001202556.1 and NM_001202557.1; Uniprot ID P16070) encoded by the CD44 molecule (Indian blood group) (CD44) gene (UniGene ID 717487).

CD103 (cluster of differentiation 103; also known as Integrin, alpha E (ITGAE)) is an adhesion-related marker and an integrin protein that in human is encoded by the ITGAE gene. CD103 binds integrin beta 7 (p7-ITGB7) to form the complete heterodimeric integrin molecule αEβ7. CD103 is expressed on various cell types including e.g., intraepithelial lymphocyte (IEL) T cells (both αβ T cells and γδ T cells) and on some peripheral regulatory T cells (Tregs). CD103 expression may be indicative of the engagement of immune cells to a cancer. Suitable antibodies targeting CD103 include, but are not limited to, the BD Horizon™ BV421 Mouse Anti-Human CD103 from BD Biosciences, the BD Pharmingen™ APC Mouse Anti-Human CD103 from BD biosciences, the APC anti-mouse CD103 Antibody anti-CD103-2E7 from BioLegend, and the like. In humans, the CD103 protein (e.g., RefSeq NP_002199.3 corresponding to NCBI reference sequence NM_002208.4; Uniprot ID P38570) is encoded by the Integrin, alpha E (antigen CD103, human mucosal lymphocyte antigen 1; alpha polypeptide) (ITGAE) gene (UniGene ID 904614).

Pan CK (pan cytokeratin) refers to a collection of cytokeratins that collectively serve as a marker of epithelial cell types and, by their absence in some instances, a mesenchymal marker. Cytokeratins as a group include at least 29 different proteins that are characteristic of epithelial and trichocytic cells. There are two types of cytokeratins the acidic type I cytokeratins and the basic or neutral type II cytokeratins. Cytokeratins are usually found in pairs comprising a type I cytokeratin and a type II cytokeratin. Anti-pan cytokeratin antibodies are broadly reactive and recognize epitopes present in most human epithelial tissues, facilitating typing of normal, metaplastic and neoplastic cells. Suitable antibodies targeting Pan CK include, but are not limited to, the Anti-pan Cytokeratin antibody [AE1/AE3+5D3] (ab86734) from Abcam Inc., the Monoclonal Anti-Cytokeratin, pan-FITC antibody produced in mouse from Millipore-Sigma, the Purified anti-Pan-Cytokeratin Antibody (Clone AE-1/AE-3) from BioLegend, the Pan-Keratin (C11) Mouse mAb (Alexa Fluor® 488 Conjugate) #4523 from Cell Signaling Technology Inc., and the like. In humans, the cytokeratin proteins may be present in various forms (e.g., RefSeq NP_005545.1, NP_258259.1, NP_001287743.1, NP_002265.2, NP_998821.3, NP_000414.2, NP_872303.2, NP_000217.2, NP_001139697.1, NP_778238.1, NP_004684.2, NP_005547.3, NP_002267.2, NP_853515.2, NP_056932.2, NP_000412.3, NP_002266.2, NP_005548.2, NP_000413.1, NP_000215.1, NP_061883.1, NP_775109.2, NP_000214.1, NP_000517.2, NP_853512.1, NP_006112.3, NP_787028.1, NP_001074961.1, NP_476429.2, NP_000415.2, NP_005546.2, NP_778223.2, NP_001243222.1, NP_061889.2, NP_778253.2, NP_853513.2, NP_853517.2 and NP_001269362.1: Uniprot ID P02538, Q3SY84, Q8N1N4, P13646, Q6A163, P35908, Q6A162, P35527, Q14CN4, 086Y46, 095678, P08729, P08727, Q7Z3Y8, Q01546, P13645, P19012, P08779, Q04695, P05783, P35900, P48668, Q99456, P02533, Q7Z3ZO, P04264, Q5XKE5, Q6KB66, P12035, P19013, P13647, P04259, Q7RTS7, P05787, Q2M215, Q7Z794, Q7Z3Y7, Q7Z3Y9 and Q9C075) encoded by various cytokeratin genes (e.g., UniGene ID Hs.700779, Hs.660007, Hs.665267, Hs.654550, Hs.28467, Hs.707, Hs.534929, Hs.654569, Hs.662013, Hs.55410, Hs.697046, Hs.411501, Hs.654568, Hs.59363, Hs.654392, Hs.99936, Hs.654570, Hs.655160, Hs.2785, Hs.406013, Hs.84905, Hs.700779, Hs.66739, Hs.654380, Hs.55412, Hs.80828, Hs.711471, Hs.140978, Hs.680652, Hs.654610, Hs.433845, Hs.708950, Hs.660125, Hs.533782, Hs.87383, Hs.334989, Hs.59736, Hs.592133, Hs.9029,).

Vimentin (also known as VIM, CTRCT30, HEL113, etc.) is a mesenchymal marker and a structural protein that in humans is encoded by the VIM gene. Vimentin is a type III intermediate filament (IF) protein that is expressed in mesenchymal cells. Vimentin is useful as a marker of mesenchymally-derived cells or cells undergoing an epithelial-to-mesenchymal transition (EMT) during both normal development and metastatic progression. Suitable antibodies targeting vimentin include, but are not limited to, the Anti-Vimentin antibody [EPR3776] from Abcam Inc., the Anti-Vimentin antibody (ab24525) from Abcam Inc., the Anti-Vimentin antibody [RV202] from Abcam Inc., the Alexa Fluor® 647 anti-Vimentin Antibody from BioLegend, and the like. In humans, the vimentin protein (e.g., RefSeq NP_003371.2 corresponding to NCB reference sequence NM_003380.3; Uniprot ID P08670) is encoded by a vimentin gene (UniGene ID 624456 or 2760449).

Useful binding members specific for any one of the above described markers include but are not limited to antibodies specific to the relevant human protein (i.e., anti-human-[marker] antibodies), including e.g., polyclonal and monoclonal antibodies produced in various host species (e.g., rabbit, mouse, goat, rat, hamster, etc.). Such antibodies, suitable for cytometric analyses, are available from various commercial suppliers including but not limited to e.g., Cell Signaling Technology, Inc. (CST, Danvers, Mass.); Thermo Fisher Scientific (Waltham, Mass.); BD Biosciences (San Jose, Calif.); BioLegend (San Diego, Calif.); Tonbo Biosciences (San Diego, Calif.); MBL International (Woburn, Mass.); LifeSpan BioSciences (Seattle, Wash.); Bio-Rad (Formerly AbD Serotec, Hercules, Calif.); Origene Technologies Inc. (Rockville, Md.); MyBioSource.com (San Diego, Calif.); Miltenyi Biotec (Bergisch Gladbach, Germany); MilliporeSigma (Burlington, Mass.); Beckman Coulter Life Sciences (Indianapolis, Ind.); Affinitylmmuno Inc. (Charlottetown, PE, Canada); ProSci, Inc. (Poway, Calif.); Bio X Cell (West Lebanon, N.H.); EXBIO Praha, a.s (Czech Republic); enQuire BioReagents (Denver, Colo.); Biogems International, Inc. (Westlake Village, Calif.); United States Biological (Salem, Mass.); R&D Systems (Minneapolis, Minn.); Santa Cruz Biotechnology (Santa Cruz, Calif.); Abcam Inc. (Cambridge, United Kingdom); and the like.

Described markers include cell surface markers. As used herein, the term “cell surface markers” refers to components of the cell that are at least exposed, partially or completely, on the outer surface of the plasma membrane of cell and thus may be accessed without modulating cell permeability, e.g., without the use of one or more permeabilizing reagents as described herein. In some instances, cell surface markers include components of the cell that have a portion exposed on the outer surface of the cell membrane but also contain an intracellular portion and/or a transmembrane portion.

Described markers include intracellular markers. As used herein, the term “intracellular markers” refers to components of the cell that are inside the cell (i.e., within the cell outer surface of the plasma membrane of the cell) and thus not exposed on the outer surface of the plasma membrane. Non-limiting examples of intracellular markers include nucleic acid markers, such as mRNA markers, DNA markers (e.g., DNA content markers, DNA as a component of a cell cycle marker, etc.), and the like. Depending on the binding member employed to detect an intracellular marker, labeling of a cell for an intracellular marker may or may not involve permeabilizing the cell with one or more permeabilization reagents. For example, in some instances one or more cell permeable binding members may be employed, such as e.g., cell permeable dyes or stains, such as but not limited to e.g., DNA dyes/stains, cytoplasmic dyes, etc., thus permeabilization may not be necessary. In some instances, one or more cell-impermeable binding members may be employed, such as e.g., cell impermeable nucleic acid or protein binding members, such as but not limited to e.g., antibodies, nucleic acid (e.g., mRNA) probes, etc., thus permeabilization may be employed.

As described above, e.g., regarding the detection of DNA content, the herein described methods may include detection of DNA using one or more DNA labeling reagents. Various DNA labeling reagents may find use in the herein described methods including but not limited to: Hoechst 33342 (2′-(4-Ethoxyphenyl)-5-(4-methyl-1-piperazinyl)-1H,1′H-2,5′-bibenzimidazole trihydrochloride) and Hoechst 33258 (4-[6-(4-Methyl-1-piperazinyl)-1′,3′-dihydro-1H,2′H-2,5′-bibenzimidazol-2′-ylidene]-2,5-cyclohexadien-1-one trihydrochloride) and others of the Hoechst series; SYTO 40, SYTO 11, 12, 13, 14, 15, 16, 20, 21, 22, 23, 24, 25 (green); SYTO 17, 59 (red), DAPI, DRAQ5™ (an anthraquinone dye with high affinity for double stranded DNA), YOYO-1, propidium iodide, YO-PRO-3, TO-PRO-3, YOYO-3 and TOTO-3, SYTOX Green, SYTOX, methyl green, acridine homodimer, 7-aminoactinomycin D, 9-amino-6-chloro-2-methoxyactridine. Depending on the particular stain and assay, the stain may serve in detection of DNA, detection of nuclei, quantitation of DNA, quantitation of nuclei, cell cycle indicator, cell proliferation indicator, DNA integrity indicator, etc.

Methods of the present disclosure may, in some instances, include the use of one or more viability dyes for various purposes. For example, in some instances, a viability dye may be employed to determine whether a subject cell is alive or dead. In some instances, a viability dye may be employed as part of an assay of a cell marker as described above, including e.g., as part of an assay of DNA content, as part of an assay of cell cycle, etc. Useful viability dyes include but are not limited to e.g., DNA dyes, DNA intercalating dyes, vial dyes, propidium iodide, calcein, Hoechst dyes, etc., as well as propidium iodide (PI), 7-amino-actinomycin D (7-AAD), and those available from commercial distributors such as Fixable Viability Dye eFluor 455UV/450/506/520/660/780 (Affymetrix eBioscience, San Diego, Calif.), LIVE/DEAD Fixable Blue/Violet/Aqua/Yellow stain (Life Technologies, Grand Island, N.Y.), Zombie Aqua/Green/NIR/RED/UV/Violet/Yellow (BioLegend, San Diego, Calif.), and the like.

As described herein and as will be readily apparent to one or ordinary skill in the art, any combination of the agents and labels described herein may be employed in the methods described provided the combination is appropriate and the components do not physically or optically interfere. For example, where alterations or substitutions of particular labels can and/or should be employed in order to allow for the combination of two or more desired components is within the skill of the ordinary artisan. As a non-limiting example, where a particular fluorescent label of a biomarker interferes optically (e.g., has an overlapping emission spectra) with a desired DNA labeling agent of a particular emission wavelength, the fluorescent label of the biomarker may be substituted with a different fluorescent label having no or less emission spectra overlap with the desired DNA labeling agent.

### Methods of Screening

As summarized above, the present disclosure includes methods of screening a subject. Such screening may include e.g., screening a subject for a metastatic cancer through the detection of one or more cell types and/or cell populations. The detected cell type(s) and/or cell population(s) may be detected based on the expression of one or more markers as described herein, including e.g., one or more immune checkpoint markers, one or more immune cell-type markers, one or more additional markers, and/or combinations thereof. Detected cell type(s) and/or cell populations useful in screening a subject for a metastatic cancer include TILs expressing combinations of immune checkpoint and immune cell-type markers.

Without being bound by theory, the inventors of the present disclosure have discovered that the presence and/or absence of certain cancer-related cell populations, such as for instance certain TIL populations, having certain marker expression profiles, and/or combinations of multiple cancer-related cell populations, is/are indicative of the presence of metastatic cancer within a subject and/or the presence of circulating tumor cells (CTCs) in the subject. In some aspects, the detection of these discovered cell populations may be practically applied to provide a technological improvement in detecting, screening for and treating subject having or at risk of having or developing a metastatic cancer and/or CTCs.

In some instances, the presence or absence of one or more cancer-related cell types or cell populations thereof may indicate the relative presence or absence of CTCs in the subject and, by corollary, the presence or absence of metastatic cancer in the subject. For example, in some instances, the presence or absence of one or more cancer-related cell types or cell populations thereof may indicate that five or fewer CTCs are likely to be detected in a CTC assay, including e.g., four or fewer CTCs, three or fewer CTCs, two or fewer CTCs, one or no CTCs, or no CTCs are likely to be detected in a CTC assay.

Subjects having an absence of one or more cancer-related cell types or cell populations thereof may be identified as not having metastatic cancer or likely not having metastatic cancer. Correspondingly, subjects having an absence of one or more cancer-related cell types or cell populations thereof may be expected to have a level of CTCs that is at or below a pre-determined threshold (such as e.g., five, four, three, two, or one CTCs), detectable by CTC assay, that indicates that the subject does not have, or likely does not have, metastatic cancer. Subjects having a level of one or more cancer-related cell types or cell populations thereof at or below a determined threshold may be identified as not having metastatic cancer or likely not having metastatic cancer. Correspondingly, subjects having one or more cancer-related cell types or cell populations thereof at or below a determined threshold may be expected to have a level of CTCs that is at or below a pre-determined threshold (such as e.g., five, four, three, two, or one CTCs), detectable by CTC assay, that indicates that the subject does not have, or likely does not have, metastatic cancer.

Subjects having one or more cancer-related cell types or cell populations thereof may be identified as having metastatic cancer or likely having metastatic cancer. Correspondingly, subjects having one or more cancer-related cell types or cell populations thereof may be expected to have a level of CTCs that is above a pre-determined threshold (such as e.g., five, four, three, two, or one CTCs), detectable by CTC assay, that indicates that the subject does have, or likely has, metastatic cancer. Subjects having a level of one or more cancer-related cell types or cell populations thereof at or above a determined threshold may be identified as having metastatic cancer or likely having metastatic cancer. Correspondingly, subjects having one or more cancer-related cell types or cell populations thereof at or above a determined threshold may be expected to have a level of CTCs that is above a pre-determined threshold (such e.g., as five, four, three, two, one), detectable by CTC assay, that indicates that the subject does have, or likely has, metastatic cancer.

Useful threshold levels of cancer-related cell types or cell populations thereof at or above which a subject may be identified as having, or likely having, metastatic cancer will vary depending on various factors, including e.g., the particular cancer-related cell type or cell population thereof assayed. Correspondingly, useful threshold levels of cancer-related cell types or cell populations thereof at or below which a subject may be identified as not having, or likely not having, metastatic cancer will similarly vary. For example, in some instances, a threshold level of a cancer-related cell population of 15% may be employed, where e.g., when the subject population represents 15% or more of the assayed cells a metastasis is or is likely present and when the subject population represents less than 15% a metastasis is not present or is likely not present. In some instances, such a threshold level may be 15% or less than 15%, including but not limited to e.g., 14% or less, 13% or less, 12% or less, 11% or less, 10% or less, 9% or less, 8% or less, 7% or less, 6% or less, 5% or less, 4% or less, 3% or less, 2.5% or less, 2% or less, 1.5% or less, 1% or less, or 0.5% or less. In some instances, the assayed cells of which the subject population is a percentage may be all of the assayed cells of the sample or a portion thereof, including but not limited to e.g., the assayed immune cells of the sample, the assayed lymphocytes of the sample, etc.

In some embodiments, a determination that a metastasis and/or CTCs is/are present or absent in a subject may include a threshold for a cancer-related cell population expressing PD-1 and CTLA-4 of 15% or less, including but not limited to e.g., 14%, 13%, 12%, 11%, 10%, 9%, 8%, 7%, 6%, 5%, 4%, 3%, 2.5%, 2%, 1.5%, 1%, or 0.5%. For example, where the cancer-related cell population expressing PD-1 and CTLA-4 represents greater than or equal to the threshold percentage of the assayed immune cells (independently or when combined with other cancer-related cell population(s) in some instances), the subject is determined to have, or likely have, a metastasis and/or CTCs. Correspondingly, where the cancer-related cell population expressing PD-1 and CTLA-4 represents less than the threshold percentage of the assayed immune cells (independently or when combined with other cancer-related cell population(s) in some instances), the subject is determined not to have, or likely not to have, a metastasis and/or CTCs.

In some embodiments, a determination that a metastasis and/or CTCs is/are present or absent in a subject may include a threshold for a cancer-related cell population expressing PD-1, CTLA-4 and CD4 of 15% or less, including but not limited to e.g., 14%, 13%, 12%, 11%, 10%, 9%, 8%, 7%, 6%, 5%, 4%, 3%, 2.5%, 2%, 1.5%, 1%, or 0.5%. For example, where the cancer-related cell population expressing PD-1, CTLA-4 and CD4 represents greater than or equal to the threshold percentage of the assayed immune cells (independently or when combined with other cancer-related cell population(s) in some instances), the subject is determined to have, or likely have, a metastasis and/or CTCs. Correspondingly, where the cancer-related cell population expressing PD-1, CTLA-4 and CD4 represents less than the threshold percentage of the assayed immune cells (independently or when combined with other cancer-related cell population(s) in some instances), the subject is determined not to have, or likely not to have, a metastasis and/or CTCs.

In some embodiments, a determination that a metastasis and/or CTCs is/are present or absent in a subject may include a threshold for a cancer-related cell population expressing PD-1, CTLA-4 and CD8 of 15% or less, including but not limited to e.g., 14%, 13%, 12%, 11%, 10%, 9%, 8%, 7%, 6%, 5%, 4%, 3%, 2.5%, 2%, 1.5%, 1%, or 0.5%. For example, where the cancer-related cell population expressing PD-1, CTLA-4 and CD8 represents greater than or equal to the threshold percentage of the assayed immune cells (independently or when combined with other cancer-related cell population(s) in some instances), the subject is determined to have, or likely have, a metastasis and/or CTCs. Correspondingly, where the cancer-related cell population expressing PD-1, CTLA-4 and CD8 represents less than the threshold percentage of the assayed immune cells (independently or when combined with other cancer-related cell population(s) in some instances), the subject is determined not to have, or likely not to have, a metastasis and/or CTCs.

In some embodiments, a determination that a metastasis and/or CTCs is/are present or absent in a subject may include a threshold for a cancer-related cell population expressing PD-1 and CD4 of 15% or less, including but not limited to e.g., 14%, 13%, 12%, 11%, 10%, 9%, 8%, 7%, 6%, 5%, 4%, 3%, 2.5%, 2%, 1.5%, 1%, or 0.5%. For example, where the cancer-related cell population expressing PD-1 and CD4 represents greater than or equal to the threshold percentage of the assayed immune cells (independently or when combined with other cancer-related cell population(s) in some instances), the subject is determined to have, or likely have, a metastasis and/or CTCs. Correspondingly, where the cancer-related cell population expressing PD-1 and CD4 represents less than the threshold percentage of the assayed immune cells (independently or when combined with other cancer-related cell population(s) in some instances), the subject is determined not to have, or likely not to have, a metastasis and/or CTCs.

In some embodiments, a determination that a metastasis and/or CTCs is/are present or absent in a subject may include a threshold for a cancer-related cell population expressing PD-1 and TIM-3 of 15% or less, including but not limited to e.g., 14%, 13%, 12%, 11%, 10%, 9%, 8%, 7%, 6%, 5%, 4%, 3%, 2.5%, 2%, 1.5%, 1%, or 0.5%. For example, where the cancer-related cell population expressing PD-1 and TIM-3 represents greater than or equal to the threshold percentage of the assayed immune cells (independently or when combined with other cancer-related cell population(s) in some instances), the subject is determined to have, or likely have, a metastasis and/or CTCs. Correspondingly, where the cancer-related cell population expressing PD-1 and TIM-3 represents less than the threshold percentage of the assayed immune cells (independently or when combined with other cancer-related cell population(s) in some instances), the subject is determined not to have, or likely not to have, a metastasis and/or

CTCs.

In some embodiments, a determination that a metastasis and/or CTCs is/are present or absent in a subject may include a threshold for a cancer-related cell population expressing PD-1, TIM-3 and CD4 of 15% or less, including but not limited to e.g., 14%, 13%, 12%, 11%, 10%, 9%, 8%, 7%, 6%, 5%, 4%, 3%, 2.5%, 2%, 1.5%, 1%, or 0.5%. For example, where the cancer-related cell population expressing PD-1, TIM-3 and CD4 represents greater than or equal to the threshold percentage of the assayed immune cells (independently or when combined with other cancer-related cell population(s) in some instances), the subject is determined to have, or likely have, a metastasis and/or CTCs. Correspondingly, where the cancer-related cell population expressing PD-1, TIM-3 and CD4 represents less than the threshold percentage of the assayed immune cells (independently or when combined with other cancer-related cell population(s) in some instances), the subject is determined not to have, or likely not to have, a metastasis and/or CTCs.

In some embodiments, a determination that a metastasis and/or CTCs is/are present or absent in a subject may include a threshold for a cancer-related cell population expressing PD-1, LAG-3 and CD4 of 15% or less, including but not limited to e.g., 14%, 13%, 12%, 11%, 10%, 9%, 8%, 7%, 6%, 5%, 4%, 3%, 2.5%, 2%, 1.5%, 1%, or 0.5%. For example, where the cancer-related cell population expressing PD-1, LAG-3 and CD4 represents greater than or equal to the threshold percentage of the assayed immune cells (independently or when combined with other cancer-related cell population(s) in some instances), the subject is determined to have, or likely have, a metastasis and/or CTCs. Correspondingly, where the cancer-related cell population expressing PD-1, LAG-3 and CD4 represents less than the threshold percentage of the assayed immune cells (independently or when combined with other cancer-related cell population(s) in some instances), the subject is determined not to have, or likely not to have, a metastasis and/or CTCs.

In some embodiments, a determination that a metastasis and/or CTCs is/are present or absent in a subject may include a threshold for a cancer-related cell population expressing TIM-3 and CD4 of 15% or less, including but not limited to e.g., 14%, 13%, 12%, 11%, 10%, 9%, 8%, 7%, 6%, 5%, 4%, 3%, 2.5%, 2%, 1.5%, 1%, or 0.5%. For example, where the cancer-related cell population expressing TIM-3 and CD4 represents greater than or equal to the threshold percentage of the assayed immune cells (independently or when combined with other cancer-related cell population(s) in some instances), the subject is determined to have, or likely have, a metastasis and/or CTCs. Correspondingly, where the cancer-related cell population expressing TIM-3 and CD4 represents less than the threshold percentage of the assayed immune cells (independently or when combined with other cancer-related cell population(s) in some instances), the subject is determined not to have, or likely not to have, a metastasis and/or CTCs.

In some embodiments, a determination that a metastasis and/or CTCs is/are present or absent in a subject may include a threshold for a cancer-related cell population expressing TIM-3, CD4 and LAG3 of 15% or less, including but not limited to e.g., 14%, 13%, 12%, 11%, 10%, 9%, 8%, 7%, 6%, 5%, 4%, 3%, 2.5%, 2%, 1.5%, 1%, or 0.5%. For example, where the cancer-related cell population expressing TIM-3, CD4 and LAG3 represents greater than or equal to the threshold percentage of the assayed immune cells (independently or when combined with other cancer-related cell population(s) in some instances), the subject is determined to have, or likely have, a metastasis and/or CTCs. Correspondingly, where the cancer-related cell population expressing TIM-3, CD4 and LAG3 represents less than the threshold percentage of the assayed immune cells (independently or when combined with other cancer-related cell population(s) in some instances), the subject is determined not to have, or likely not to have, a metastasis and/or CTCs.

In some embodiments, a determination that a metastasis and/or CTCs is/are present or absent in a subject may include a threshold for a cancer-related cell population expressing TIM-3 and CTLA-4 of 15% or less, including but not limited to e.g., 14%, 13%, 12%, 11%, 10%, 9%, 8%, 7%, 6%, 5%, 4%, 3%, 2.5%, 2%, 1.5%, 1%, or 0.5%. For example, where the cancer-related cell population expressing TIM-3 and CTLA-4 represents greater than or equal to the threshold percentage of the assayed immune cells (independently or when combined with other cancer-related cell population(s) in some instances), the subject is determined to have, or likely have, a metastasis and/or CTCs. Correspondingly, where the cancer-related cell population expressing TIM-3 and CTLA-4 represents less than the threshold percentage of the assayed immune cells (independently or when combined with other cancer-related cell population(s) in some instances), the subject is determined not to have, or likely not to have, a metastasis and/or CTCs.

In some embodiments, a determination that a metastasis and/or CTCs is/are present or absent in a subject may include a threshold for a cancer-related cell population expressing TIM-3, CTLA-4 and CD4 of 15% or less, including but not limited to e.g., 14%, 13%, 12%, 11%, 10%, 9%, 8%, 7%, 6%, 5%, 4%, 3%, 2.5%, 2%, 1.5%, 1%, or 0.5%. For example, where the cancer-related cell population expressing TIM-3, CTLA-4 and CD4 represents greater than or equal to the threshold percentage of the assayed immune cells (independently or when combined with other cancer-related cell population(s) in some instances), the subject is determined to have, or likely have, a metastasis and/or CTCs. Correspondingly, where the cancer-related cell population expressing TIM-3, CTLA-4 and CD4 represents less than the threshold percentage of the assayed immune cells (independently or when combined with other cancer-related cell population(s) in some instances), the subject is determined not to have, or likely not to have, a metastasis and/or CTCs.

In some embodiments, a determination that a metastasis and/or CTCs is/are present or absent in a subject may include a threshold for a cancer-related cell population expressing TIM-3, CTLA-4 and CD8 of 15% or less, including but not limited to e.g., 14%, 13%, 12%, 11%, 10%, 9%, 8%, 7%, 6%, 5%, 4%, 3%, 2.5%, 2%, 1.5%, 1%, or 0.5%. For example, where the cancer-related cell population expressing TIM-3, CTLA-4 and CD8 represents greater than or equal to the threshold percentage of the assayed immune cells (independently or when combined with other cancer-related cell population(s) in some instances), the subject is determined to have, or likely have, a metastasis and/or CTCs. Correspondingly, where the cancer-related cell population expressing TIM-3, CTLA-4 and CD8 represents less than the threshold percentage of the assayed immune cells (independently or when combined with other cancer-related cell population(s) in some instances), the subject is determined not to have, or likely not to have, a metastasis and/or CTCs.

In some embodiments, a determination that a metastasis and/or CTCs is/are present or absent in a subject may include a threshold for a cancer-related cell population expressing TIM-3 and an NK cell marker of 15% or less, including but not limited to e.g., 14%, 13%, 12%, 11%, 10%, 9%, 8%, 7%, 6%, 5%, 4%, 3%, 2.5%, 2%, 1.5%, 1%, or 0.5%. For example, where the cancer-related cell population expressing TIM-3 and an NK cell marker represents greater than or equal to the threshold percentage of the assayed immune cells (independently or when combined with other cancer-related cell population(s) in some instances), the subject is determined to have, or likely have, a metastasis and/or CTCs. Correspondingly, where the cancer-related cell population expressing TIM-3 and an NK cell marker represents less than the threshold percentage of the assayed immune cells (independently or when combined with other cancer-related cell population(s) in some instances), the subject is determined not to have, or likely not to have, a metastasis and/or CTCs.

In some embodiments, a determination that a metastasis and/or CTCs is/are present or absent in a subject may include a threshold for a cancer-related cell population expressing LAG-3 and CD4 of 15% or less, including but not limited to e.g., 14%, 13%, 12%, 11%, 10%, 9%, 8%, 7%, 6%, 5%, 4%, 3%, 2.5%, 2%, 1.5%, 1%, or 0.5%. For example, where the cancer-related cell population expressing LAG-3 and CD4 represents greater than or equal to the threshold percentage of the assayed immune cells (independently or when combined with other cancer-related cell population(s) in some instances), the subject is determined to have, or likely have, a metastasis and/or CTCs. Correspondingly, where the cancer-related cell population expressing LAG-3 and CD4 represents less than the threshold percentage of the assayed immune cells (independently or when combined with other cancer-related cell population(s) in some instances), the subject is determined not to have, or likely not to have, a metastasis and/or CTCs.

In some embodiments, a determination that a metastasis and/or CTCs is/are present or absent in a subject may include a threshold for a cancer-related cell population expressing LAG-3, CD4 and CTLA-4 of 15% or less, including but not limited to e.g., 14%, 13%, 12%, 11%, 10%, 9%, 8%, 7%, 6%, 5%, 4%, 3%, 2.5%, 2%, 1.5%, 1%, or 0.5%. For example, where the cancer-related cell population expressing LAG-3, CD4 and CTLA-4 represents greater than or equal to the threshold percentage of the assayed immune cells (independently or when combined with other cancer-related cell population(s) in some instances), the subject is determined to have, or likely have, a metastasis and/or CTCs. Correspondingly, where the cancer-related cell population expressing LAG-3, CD4 and CTLA-4 represents less than the threshold percentage of the assayed immune cells (independently or when combined with other cancer-related cell population(s) in some instances), the subject is determined not to have, or likely not to have, a metastasis and/or CTCs.

In some embodiments, a determination that a metastasis and/or CTCs is/are present or absent in a subject may include a threshold for a cancer-related cell population expressing HLA-DR of 15% or less, including but not limited to e.g., 14%, 13%, 12%, 11%, 10%, 9%, 8%, 7%, 6%, 5%, 4%, 3%, 2.5%, 2%, 1.5%, 1%, or 0.5%. For example, where the cancer-related cell population expressing HLA-DR represents greater than or equal to the threshold percentage of the assayed immune cells (independently or when combined with other cancer-related cell population(s) in some instances), the subject is determined to have, or likely have, a metastasis and/or CTCs. Correspondingly, where the cancer-related cell population expressing HLA-DR represents less than the threshold percentage of the assayed immune cells (independently or when combined with other cancer-related cell population(s) in some instances), the subject is determined not to have, or likely not to have, a metastasis and/or CTCs.

In some embodiments, a determination that a metastasis and/or CTCs is/are present or absent in a subject may include a threshold for a cancer-related cell population expressing HLA-DR and CD103 of 15% or less, including but not limited to e.g., 14%, 13%, 12%, 11%, 10%, 9%, 8%, 7%, 6%, 5%, 4%, 3%, 2.5%, 2%, 1.5%, 1%, or 0.5%. For example, where the cancer-related cell population expressing HLA-DR and CD103 represents greater than or equal to the threshold percentage of the assayed immune cells (independently or when combined with other cancer-related cell population(s) in some instances), the subject is determined to have, or likely have, a metastasis and/or CTCs. Correspondingly, where the cancer-related cell population expressing HLA-DR and CD103 represents less than the threshold percentage of the assayed immune cells (independently or when combined with other cancer-related cell population(s) in some instances), the subject is determined not to have, or likely not to have, a metastasis and/or CTCs.

In some embodiments, a determination that a metastasis and/or CTCs is/are present or absent in a subject may include a threshold for a cancer-related cell population of immune cells expressing CCR5, including e.g., Treg cells expressing CCR5, of 15% or less, including but not limited to e.g., 14%, 13%, 12%, 11%, 10%, 9%, 8%, 7%, 6%, 5%, 4%, 3%, 2.5%, 2%, 1.5%, 1%, or 0.5%. For example, where the cancer-related cell population expressing CCR5 represents greater than or equal to the threshold percentage of the assayed immune cells (independently or when combined with other cancer-related cell population(s) in some instances), the subject is determined to have, or likely have, a metastasis and/or CTCs. Correspondingly, where the cancer-related cell population expressing CCR5 represents less than the threshold percentage of the assayed immune cells (independently or when combined with other cancer-related cell population(s) in some instances), the subject is determined not to have, or likely not to have, a metastasis and/or CTCs.

In some embodiments, a determination that a metastasis and/or CTCs is/are present or absent in a subject may include a threshold for a cancer-related cell population of immune cells expressing at least one immune checkpoint marker and CCR5, including e.g., Treg cells expressing CCR5, of 15% or less, including but not limited to e.g., 14%, 13%, 12%, 11%, 10%, 9%, 8%, 7%, 6%, 5%, 4%, 3%, 2.5%, 2%, 1.5%, 1%, or 0.5%. For example, where the cancer-related cell population expressing at least one immune checkpoint marker and CCR5 represents greater than or equal to the threshold percentage of the assayed immune cells (independently or when combined with other cancer-related cell population(s) in some instances), the subject is determined to have, or likely have, a metastasis and/or CTCs. Correspondingly, where the cancer-related cell population expressing at least one immune checkpoint marker and CCR5 represents less than the threshold percentage of the assayed immune cells (independently or when combined with other cancer-related cell population(s) in some instances), the subject is determined not to have, or likely not to have, a metastasis and/or CTCs.

The above represent non-limiting examples of threshold levels of particular cancer-related cell populations above or below which the presence or absence of the population may indicate, alone or in combination with other cancer-related cell populations, the presence or absence of metastatic cancer and/or CTCs. These examples are not limiting and, in some instances, other cancer-related cell populations may be employed with similar and/or corresponding thresholds in the assays of the present disclosure.

In some instances, e.g., where multiple different cancer-related cell populations are employed in screening for a metastatic condition and/or the presence/absence of CTCs, different thresholds for each different cancer-related cell population may be employed. For example, a first threshold level of 2% for a first cancer-related cell population and a second threshold level of 12% for a second cancer-related cell population may be employed, such that detection of greater than 2% of the first cancer-related cell population and/or greater than 12% of the second cancer-related cell population in an assay indicates that a metastasis is, or is likely, present.

In some instances, the same threshold may be employed for two or more different cancer-related cell populations. For example, in some instances, a single threshold, e.g., a threshold of 1%, may be employed for multiple populations such that e.g., the presence of the multiple populations all at or above 1% indicates presence of metastasis and/or CTCs and the presence of the multiple populations all below 1% indicates absence of metastasis and/or CTCs. Where the same threshold is employed for multiple different populations the actual threshold used may vary depending on the populations and the context and may include but is not limited to e.g., 15% or less, including but not limited to e.g., 14%, 13%, 12%, 11%, 10%, 9%, 8%, 7%, 6%, 5%, 4%, 3%, 2.5%, 2%, 1.5%, 1%, or 0.5%.

As described herein, detection of individual cancer-related cell types and/or populations thereof may be employed alone or in combination in making a determination about the presence/absence of metastasis. As such, in some instances, detection of a single cancer-related cell type and/or population thereof above or below a particular threshold may be employed in making a determination about the presence/absence of metastasis and/or CTCs. In some instances, detection of a plurality of different cancer-related cell types and/or populations thereof above or below particular thresholds may be employed in making a determination about the presence/absence of metastasis and/or CTCs. For example, in some instances, a determination that a metastasis and/or CTCs is/are present may require that two or more (including e.g., three or more, four or more, five or more, six or more, etc.) different cancer-related cell types and/or populations thereof are all at or above their respective threshold levels. Correspondingly, a determination that a metastasis and/or CTCs is/are present may require that two or more (including e.g., three or more, four or more, five or more, six or more, etc.) different cancer-related cell types and/or populations thereof are all at or below their respective threshold levels.

In some instances, by combining a plurality of different cancer-related cell types and/or populations thereof into a metastasis/CTC determination greater predictive power may be achieved. For example, an assay employing a plurality of different cancer-related cell types and/or populations thereof may better predict the presence/absence of metastasis and/or CTCs than assaying the individual cancer-related cell types and/or populations thereof otherwise would. In some instances, a combination of a plurality of different cancer-related cell types and/or populations thereof for use in making a metastasis/CTC determination may include one or more cancer-related cell types and/or populations thereof and/or detection thresholds thereof that may not independently discriminate between the presence and absence of metastasis and/or CTCs alone. In some instances, a cancer-related cell type and/or population thereof and/or detection threshold thereof may independently discriminate between the presence and absence of metastasis and/or CTCs.

Numerous useful thresholds described herein include where the cancer-related cell population represents 15% or less of the relevant assayed cells (e.g., immune cells, lymphocytes, etc.). However, such should not be considered limiting and, in some instances, one or more thresholds above 15% may be employed, including but not limited to e.g., 20%, 25%, 30%, 35%, 40%, 45%, 50%, etc. Such higher thresholds may find use in various circumstances, including e.g., where the relevant group of assayed cells (of which the cancer-related cell population is a part) is relatively small and/or specific. For example, when the relevant assayed cells of which the cancer-related population is a part is limited to CD4+ cells, CD8+ cells, NK cells, Treg cells, and the like.

Accordingly, the methods of screening provided in the present disclosure may include detecting one or more cancer-related cell types or cell populations thereof, including e.g., the methods of detecting such cell types and populations described above. The subject screening methods may or may not include a testing step that follows the screening procedure and may be dependent on the outcome of the screening procedure. For example, a subject method may include detecting whether a particular TIL population is present in a sample from a subject and then further testing the subject for a metastatic cancer based on the outcome of the screening, e.g., testing the subject when presence of the TIL population is detected.

Testing of a subject, e.g., useful to determine and/or confirm whether a metastatic cancer is present, will vary. For example, in some instances, testing of a subject may include obtaining a sample from the subject for testing. Useful samples for testing will vary and may include e.g., liquid samples (e.g., blood, lymph or the like), tissue samples, biopsy samples (e.g., tumor biopsy samples, lymph node biopsy samples, etc.), and the like. In some instances, testing of a subject may include a lymph node biopsy, including a biopsy of one or multiple lymph nodes of a subject, such as sentinel lymph node biopsy, a regional lymph node biopsy, and the like. In some instances, testing of a subject may include medical imaging, including but not limited to e.g., imaging employed to detect metastatic cancer.

In some instances, testing of a subject may include an assay to detect and/or confirm the presence/absence of circulating tumor cells (CTCs), referred to herein as a circulating tumor cell (CTC) assay. In some instances, a liquid sample obtained from a subject may be subjected to a CTC assay, including where the liquid sample is obtained and/or subjected to the CTC assay only when one or more cancer-related cell populations are first assayed for and detected, e.g., as described above.

In some instances, a CTC assay may include assaying one or more markers for a CTC. As used herein, the term “CTC” generally refers to those neoplastic cells that have sloughed off of a tumor (e.g., the edge of a tumor) and have been swept away by the bloodstream or lymphatic system thus causing the CTC to circulate in the body. CTC makers include e.g., those markers used in identifying CTCs in the blood stream including but not limited to e.g., Epithelial cell adhesion molecule (EpCAM), cytokeratin 8, cytokeratin 18 and cytokeratin 19. In some instances, CTCs arising from non-immune related cancers may be further characterized as being negative for one or more immune cell markers, including but not limited to e.g., one or more of those immune cell markers described herein. For example, in some instances, a detected CTC may be negative for CD45. In some instances, CTCs arising from an immune related cancer may be further characterized as being positive for one or more immune cell markers, including but not limited to e.g., one or more of those immune cell markers described herein.

In some instances, CTCs may be further identified and/or characterized based on the expression of one or more cancer antigens and/or one or more cancer associated antigens. Non-limiting examples of cancer antigens include but are not limited to e.g., CD19, CD20, CD38, CD30, Her2/neu, ERBB2, CA125, MUC-1, prostate-specific membrane antigen (PSMA), CD44 surface adhesion molecule, mesothelin, carcinoembryonic antigen (CEA), epidermal growth factor receptor (EGFR), EGFRvIII, vascular endothelial growth factor receptor-2 (VEGFR2), high molecular weight-melanoma associated antigen (HMW-MAA), MAGE-A1, IL-13R-a2, GD2, and the like. Cancer-associated antigens also include, e.g., 4-1 BB, 5T4, adenocarcinoma antigen, alpha-fetoprotein, BAFF, B-lymphoma cell, C242 antigen, CA-125, carbonic anhydrase 9 (CA-IX), C-MET, CCR4, CD152, CD19, CD20, CD200, CD22, CD221, CD23 (IgE receptor), CD28, CD30 (TNFRSF8), CD33, CD4, CD40, CD44 v6, CD51, CD52, CD56, CD74, CD80, CEA, CNT0888, CTLA-4, DRS, EGFR, EpCAM, CD3, FAP, fibronectin extra domain-B, folate receptor 1, GD2, GD3 ganglioside, glycoprotein 75, GPNMB, HER2/neu, HGF, human scatter factor receptor kinase, IGF-1 receptor, IGF-I, IgG₁, L1-CAM, IL-13, IL-6, insulin-like growth factor I receptor, integrin α5β1, integrin αvβ3, MORAb-009, MS4A1, MUC1, mucin CanAg, N-glycolylneuraminic acid, NPC-1C, PDGF-R a, PDL192, phosphatidylserine, prostatic carcinoma cells, RANKL, RON, ROR1, SCH 900105, SDC1, SLAMF7, TAG-72, tenascin C, TGF beta 2, TGF-β, TRAIL-R1, TRAIL-R2, tumor antigen CTAA16.88, VEGF-A, VEGFR-1, VEGFR2, and vimentin.

Useful CTC assays include but are not limited to e.g., microfluidic CTC assays. Various microfluidic methods of isolation and characterization of CTCs have been developing, including but not limited to e.g., those described in Yu et al. (Journal of Cell Biology (2011) 192, 373-382); Esmaeilsabzali et al. (Biotechnology Advances (2013) 31, 1063-1084); and Zhang et al. (Cancer Biology & Therapy (2016) 17:11, 1177-1187), the disclosures of which are incorporated herein by reference in their entirety. In some instances, a CTC assay may involve a system, device and/or one or more reagents for CTC detection and/or isolation available from: Vortex Biosciences (Menlo Park, Calif.), such as the VTX-1 system and related reagents; Clearbridge BioMedics (Singapore) such as the ClearCell® FX1 System and related reagents; Celsee, Inc. (Plymouth, Mich.) systems and related reagents; CellMax Life (Sunnyvale, Calif.), such as the CellMax biomimetic platform (CMx) and related reagents; and the like.

As summarized above, a testing procedure, e.g., to verify the results of a detection assay as described herein, may be employed according to any convenient and appropriate method depending on the result to be verified. For example, where a detection assay indicates the presence of CTCs in the subject, a subject may be recommended for or provided with further testing to verify the presence of the CTCs. Correspondingly, in some instances, where a detection assay indicates the absence of CTCs in the subject, a subject may be recommended for or provided with further testing to verify the absence of the CTCs. Where a detection assay indicates the presence/absence of metastatic cancer, a subject may be recommended for or provided with further testing to verify the presence/absence of the metastatic cancer, including e.g., where such further testing includes one or more metastatic cancer tests, such as medical imaging, biopsy (including primary, secondary, and/or lymph node biopsy), and the like.

### Methods of Treating a Subject

As summarized above, the present disclosure includes methods of treating a subject for a neoplasia. The terms “subject,” “individual,” “host,” and “patient,” are used interchangeably herein and refer to any mammalian subject for whom diagnosis, treatment, or therapy is desired, particularly humans.

The subject methods may include detecting the presence or absence of one or more cell types and/or cell populations in a sample from a subject and treating the subject based on the detected presence or absence of the one or more cell types and/or cell populations. For example, in some instances, the presence or absence of a TIL population, including e.g., a TIL population having a particular marker expression of a particular combination of marker expression, may be detected and the subject may be treated based on the detected presence or absence of the TIL population.

In some instances, the mode of treatment employed may be based on whether one or more particular cell types and/or cell populations are detected. For example, in some instances, when the presence or absence of a particular cell type and/or cell population is detected, a subject may be treated with a systemic treatment. In some instances, when the presence or absence of a particular cell type and/or cell population is detected, a subject may be treated with a local treatment.

By “systemic treatment”, as used herein, is meant a treatment that is not directed solely to target a specific tumor (such as e.g., a primary tumor or a defined secondary tumor) or a specific cancer containing tissue (such as e.g., the liver in the case of liver cancer, the blood in the case of a blood cancer, etc.). Systemic treatments will generally be directed to the subject's body as a whole and may include but are not limited to e.g., systemic radiation therapy, systemic chemotherapy, systemic immunotherapy, combinations thereof and the like.

Systemic therapies may be administered to a subject before, during or after a subject is assessed for the presence of a cancer-related cell type or population thereof will vary depending on numerous factors including e.g., the type of neoplasia, the subject's medical history, general state of health and/or any co-morbidities, and the like. In some instances, a systemic therapy may be administered based on a detected lack of a cancer-related cell type or population thereof, such as a TIL population, detected according to the methods described herein. Put another way, one or more systemic treatments may be administered when a TIL population, e.g., expressing a marker or a combination thereof, is absent. Useful systemic therapies that may be administered, e.g., following an assessment of the present disclosure, include but are not limited to e.g., systemic radiation therapy, chemotherapy, immunotherapy, combinations thereof and the like.

By “local treatment”, as used herein, is meant a treatment that is specifically directed to the location of a tumor (such as e.g., a primary tumor or a defined secondary tumor) or specifically directed to a cancer containing tissue (such as e.g., the liver in the case of liver cancer, the blood in the case of a blood cancer, etc.). In some instances, local treatment may also be administered in such a way as to affect the environment surrounding a tumor, such as tissue surrounding the tumor, such as tissue immediately adjacent to the tumor. Local treatment will generally not affect or not be targeted to tissues distant from the site of cancer including the site of a tumor, such as a primary tumor. Useful local treatments that may be administered, e.g., following an assessment of the present disclosure, include but are not limited to surgery, local radiation therapy, local cryotherapy, local laser therapy, local topical therapy, combinations thereof, and the like. In some instances, local therapies may also include targeted therapies applied locally, (i.e., targeted therapies that are not applied or do not affect a subject systemically).

Targeted therapy, as it relates to cancer, may include a treatment targeting a cancer's specific genes, proteins, or the tissue environment that contributes to the cancer growth and survival. Examples of targeted therapies include, but are not limited to e.g., drugs against EGFR (such as Afatinib), and ALK (such as Alectinib). EGFR is a receptor tyrosine kinase expressed on the cell surface that activates downstream intracellular signaling to promote cell proliferation. Frequent amplification, over-expression and/or mutation of the EGFR gene in NSCLC cells can lead to hyper-activation of cancer cell growth. ALK is another receptor tyrosine kinase that is often aberrant in NSCLC. Approximately 3-7% of lung cancers harbor ALK fusions. ALK fusions lead to the over-activation of ALK to promote cancer growth. In most cases, ALK rearrangements are not found overlapping with other oncogenic mutations found in NSCLC.

Immunotherapy as a treatment in the herein described methods may be applied locally or systemically, e.g., depending on the route of administration and/or the particular formulation of the therapeutic. Routes of local administration, depending on the context, may include e.g., direct injection, topical administration, inhalation, etc. Routes of systemic administration, depending on the context, may include e.g., intravenous administration, oral administration, etc.

Immunotherapy is as a treatment option for various cancers, including lung cancer. The PD-1 and PD-L1 pathway may be targeted for NSCLC immunotherapy. PD-1, also known as CD279, is an immune check point marker that is expressed on the surface of activated T, B, and NK cells. PD-L1 is the major ligand of PD-1 and is upregulated in many cancer types, including NSCLC. PD-1/PD-L1 pathway activation down-regulates immune responses. Cancers utilize this pathway to suppress an immune response directed to the cancer. Antibodies against PD-1, e.g., Nivolumab (Opdivo) and pembrolizumab (Keytruda), and antibodies against PD-L1, e.g., Atezolizumab (Tecentriq), which have been approved by FDA for NSCLC treatment, were developed to successfully block the PD-1 and PD-L1 interaction.

Immunotherapies also include anti-PD-1/PD-L1 immunotherapies which include but are not limited to e.g., those therapies that include administering to a subject an effective amount of one or more anti-PD-1/PD-L1 therapeutic antagonists where such antagonists include but are not limited to e.g., OPDIVO® (nivolumab), KEYTRUDA® (pembrolizumab), Tecentriq™ (atezolizumab), durvalumab (MEDI4736), avelumab (MSB0010718C), BMS-936559 (MDX-1105), CA-170, BMS-202, BMS-8, BMS-37, BMS-242 and the like.

Nivolumab (OPDIVO®) is a humanized IgG₄ anti-PD-1 monoclonal antibody used to treat cancer. Pembrolizumab (KEYTRUDA®), formerly known as MK-3475, lambrolizumab, etc., is a humanized antibody used in cancer immunotherapy targeting the PD-1 receptor. Atezolizumab (Tecentriq™) is a fully humanized, engineered monoclonal antibody of IgG₁ isotype against the PD-L1 protein. Durvalumab (MedImmune) is a therapeutic monoclonal antibody that targets PD-L1. Avelumab (also known as MSB0010718C; Merck KGaA, Darmstadt, Germany & Pfizer) is a fully human monoclonal PD-L1 antibody of isotype IgG₁. BMS-936559 (also known as MDX-1105; Bristol-Myers Squibb) is a blocking antibody that has been shown to bind to PD-L1 and prevent its binding to PD-1 (see e.g., U.S. NIH Clinical Trial No. NCT00729664). CA-170 (Curis, Inc.) is a small molecule PD-L1 antagonist. BMS-202, BMS-8, BMS-37, BMS-242 are small molecule PD-1/PD-L1 complex antagonists that bind PD-1 (see e.g., Kaz et al., (2016) Oncotarget 7(21); the disclosure of which is incorporated herein by reference in its entirety).

Useful anti-PD-L1 antagonists, including e.g., antibodies, include but are not limited to e.g., those described in U.S. Pat. Nos. 7,722,868; 7,794,710; 7,892,540; 7,943,743; 8,168,179; 8,217,149; 8,354,509; 8,383,796; 8,460,927; 8,552,154; 8,741,295; 8,747,833; 8,779,108; 8,952,136; 8,981,063; 9,045,545; 9,102,725; 9,109,034; 9,175,082; 9,212,224; 9,273,135 and 9,402,888; the disclosures of which are incorporated herein by reference in their entirety.

Useful anti-PD-1 antagonists, including e.g., antibodies, include but are not limited to e.g., those described in U.S. Pat. Nos. 6,808,710; 7,029,674; 7,101,550; 7,488,802; 7,521,051; 8,008,449; 8,088,905; 8,168,757; 8,460,886; 8,709,416; 8,951,518; 8,952,136; 8,993,731; 9,067,998; 9,084,776; 9,102,725; 9,102,727; 9,102,728; 9,109,034; 9,181,342; 9,205,148; 9,217,034; 9,220,776; 9,308,253; 9,358,289; 9,387,247 and 9,402,899; the disclosures of which are incorporated herein by reference in their entirety.

Other immune check point markers, such as CTLA-4, LAG-3 and TIM-3, may be targeted in the subject methods of treatment. CTLA-4, also known as CD152, binds to CD80 and CD86. Antibodies against CTLA-4 have been approved for treating some cancer types. The co-inhibitory effect of CTLA-4 and PD-1 make CTLA-4 a good candidate for use in in combination with anti-PD-1 to treat certain cancers. LAG-3 is in clinical trials for treating cancers, including e.g., NSCLC. TIM-3 may also be targeted for immunotherapy for several cancer types, including e.g., NSCLC.

Antagonist LAG-3 antibodies can both activate T effector cells (by downregulating the LAG-3 inhibiting signal into pre-activated LAG-3+ cells) and inhibit induced (i.e. antigen-specific) Treg suppressive activity. Useful LAG-3 antagonistic antibodies include relatlimab (BMS-986016; developed by Bristol-Myers Squibb), IMP701 (developed by Immutep), TSR-033 (anti-LAG-3 mAb; developed by TESARO, Inc.), and the like.

Immunotherapies also include T cell-based immunotherapies such as e.g., adoptive cell therapy (ACT) and chimeric antigen receptor (CAR) T cell therapies. For example, a subject may be administered a population of CAR T cells engineered to target an antigen expressed by the subject's cancer. A T cell-based therapy may involve, in some instances, obtaining a cellular sample from a subject, such as a blood sample or a tumor biopsy, and culturing immune cells from the sample ex vivo, with or without genetic modification of the cultured immune cells. As an example, in the case of a lung cancer, immune cells may be obtained from a subject, cultured ex vivo and modified with a CAR specific for an antigen expressed by the lung cancer, such as e.g., mesothelin, to produce a population of CAR T cells. Then, the CAR T cells may be reintroduced into the subject to target the lung cancer. T cell-based immunotherapies may be configured in various ways, e.g., by targeting various antigens, by collecting/culturing various cell types, etc., depending on a particular cancer to be treated. In addition, T cell-based immunotherapies may be administered systemically, e.g., by intravenous injection, or locally, e.g., by infusion (e.g., intraperitoneal infusion, pleural catheter infusion, etc.), direct injection, and the like.

Useful therapies may also include those targeting CCR5 expressing cells, including e.g., where a CCR5 expressing cell population is detected a CCR5 targeting therapy may be employed. CCR5 targeting therapies, as well as methods of detecting CCR5 expressing cancer cells are described in U.S. Patent Application Pub. Nos. 1 20180303830, 20170231991, 20140109245, and 20130303512; the disclosures of which are incorporated herein by reference in their entirety.

Useful drugs targeting CCR5 may, in some instances, include CCR5 antagonists, such as but not limited to e.g., small molecule (including peptide and non-peptide small molecule) inhibitors, antibodies, and the like. Non-limiting examples of CCR5 antagonists include: Maraviroc (aka Selzentry or Celsentri), INCB-9471 ((4,6-dimethylpyrimidin-5-yl)-[4-[(3S)-4-[(1R,2R)-2-ethoxy-5-(trifluoromethyl)-2,3-dihydro-1H-inden-1-yl]-3-methylpiperazin-1-yl]-4-methylpiperidin-1-yl]methanone; orally available CCR5 antagonist; PubChem CID: 49871007), Leronlimab (PRO-140, a humanized monoclonal antibody directed against CCR5), Aplaviroc (4-(4-{[(3R)-1-butyl-3-[(R)-cyclohexyl(hydroxy)methyl]-2,5-dioxo-1,4,9-triazaspiro[5.5]undecan-9-yl]methyl}phenoxy)benzoic acid; a potent noncompetitive allosteric antagonist of the CCR5 receptor), Vicriviroc (5-{4-[(3S)-4-[(1R)-2-methoxy-1-[4-(trifluoromethyl)-phenyl]ethyl]-3-methylpiperazin-1-yl]-4-methylpiperidine-1-carbonyl}-4,6-dimethylpyrimidine; CCR5 entry inhibitor previously named SCH 417690 and SCH-D), fully human monoclonal antibodies to CCR5 (such as the HGS004 as described by Lalezari et al., J Infect Dis. (2008) 197(5):721-7), and the like.

Compositions may be administered once per day, a few or several times per day, or even multiple times per day, depending upon, among other things, the indication being treated and the judgment of the prescribing physician. For example, in some instances, compositions that include one or more immunotherapy agents may be administered once per day, a few or several times per day, or even multiple times per day, depending upon, among other things, the indication being treated and the judgment of the prescribing physician.

In some instances, a subject may be assessed before a course of therapy is begun. For example, in some instances, a medical professional may assay a subject to determine whether the subject contains a cancer-related cell type or a population thereof, e.g., a TIL population, according to the methods described herein prior to administering a therapy and the medical professional may administer the particular therapy (e.g., systemic or local therapy) only if the cancer-related cell type or the population thereof is or is not identified, as appropriate.

The amount of time before starting a course of treatment at which point a subject may be assessed may vary and may range from 1 day or less to a month or more including but not limited to e.g., 1 day, 2 days, 3 days, 4 days, 5 days, 6 days, 1 week, 2 weeks, 3 weeks, 1 month, etc. In some instances, a course of treatment may be begun the same day that an assessment is performed.

In some instances, a subject may be assessed for the presence of a cancer-related cell type or population thereof, e.g., TIL population, after a course of treatment has already begun or after a course of treatment has already been administered. For example, in some instances, a subject's neoplasia may be assayed during a course of therapy, e.g., as part of a monitoring regimen, and the course of therapy may be altered based on the outcome of the assessment. For example, a subject undergoing a course of local treatment for a neoplasia may be assessed and, if the assessment detects the presence of the cancer-related cell type or population thereof, the course of therapy may be altered or amended, e.g., by stopping the course of local treatment, by beginning a course of systemic treatment in addition to the local treatment, by beginning a course of systemic treatment in place of the local treatment, etc.

In another example, a subject undergoing a course of systemic treatment for a neoplasia may be assessed and, if the assessment detects the absence of the cancer-related cell type or population thereof, the course of therapy may be altered or amended, e.g., by stopping the course of systemic treatment, by beginning a course of local treatment in addition to the systemic treatment, by beginning a course of local treatment in place of the systemic treatment, etc.

In some instances, a subject's neoplasia may be assayed after a course of therapy, e.g., as a means of determining whether further therapy is warranted and/or what type of further therapy may be warranted. Accordingly, in some instances, further therapy may or may not be administered based on the outcome of the assessment. In some instances, assays performed after a therapy has been administered may be a means of monitoring for a progression or change in disease state.

The amount of time after a course of treatment has ended that a subject may be assessed may vary and may range from 1 day or less to a month or more including but not limited to e.g., 1 day, 2 days, 3 days, 4 days, 5 days, 6 days, 1 week, 2 weeks, 3 weeks, 1 month, etc. In some instances, an assessment is performed the same day on which the course of therapy is ended. In some instances, an assessment may be performed during a long-term follow-up assessment of a subject. The length of time after a course of treatment at which point long-term follow-up is performed will vary and may range from 3 months or less to 10 years or more including but not limited to e.g., 3 months, 4 months, 5 months, 6 months, 7 months, 8 months, 9 months, 10 months, 11 months, one year, 1.5 years, 2 years, 3 years, 4 years, 5 years, 6 years, 7 years, 8 years, 9 years, 10 years, etc.

Methods of treatment described herein may be targeted to various cell populations, including e.g., one or more cell populations identified according to a method of the present disclosure. Essentially any cell type and/or cell population described herein may be targeted by an appropriate therapy. For example, in some instances, a PD-1 or PD-L1 expressing cell type or cell population may be targeted, e.g., using an anti-PD-1/PD-L1 immunotherapy.

In some instances, a LAG-3 expressing cell type or cell population may be targeted, e.g., using an anti-LAG-3 therapy, such as but not limited to an anti-LAG-3 antibody, such as e.g., relatlimab, TSR-033, or IMP701 as described above. In some embodiments, a targeted LAG-3 expressing cell population may be a LAG-3 expressing Treg population, such as but not limited to e.g., a CD4±/CD103±/LAG3+ Treg population.

In some instances, a CCR5 expressing cell type or cell population may be targeted, e.g., using a CCR5 targeting therapy, including but not limited to e.g., one or more of the various CCR5 targeting therapies described above. In some embodiments, a targeted CCR5 expressing cell population may be a CCR5 expressing FOXP3-expressing population (including e.g., a CCR5 expressing FOXP3-expressing Treg population), such as but not limited to e.g., a CD4±/CD103±/FOXP3±/CCR5+ population.

Depending on whether systemic and/or local treatment is chosen, methods of administration may be chosen depending also on the condition being treated and the pharmaceutical composition being administered. Administration of an effective amount (in one or multiple doses) of the subject agent(s) can be done in a variety of ways, including, but not limited to, subcutaneously, intravenously, intraperitoneally, intramuscularly, and direct injection to specified organs or tumors, systemic administration, etc. Administration of the pharmaceutical compositions may be through a single route or concurrently by several routes.

By “effective amount” is meant an amount sufficient to have a therapeutic effect. An effective amount that will treat a neoplasia will modulate the symptoms and/or the size and/or the growth rate of the neoplasia typically by at least about 1%, including but not limited to e.g., at least about 10%; at least about 20%; at least about 30%; at least about 50%. Such will result in, e.g., statistically significant and quantifiable changes in the numbers of cells being affected. This may be a decrease in the size of the primary tumor, a decrease in the growth rate of the primary tumor, a decrease in the growth rate of one or more metastases, a decrease in the numbers of metastases in distant organs, a decrease in recurrent metastatic disease, etc.

Methods of treating a subject for a neoplasia may, in some instances, include testing the subject, e.g., to detect and/or confirm the presence or absence of a metastasis and/or the presence or absence of CTCs. For example, in some instances, a subject may be tested based on the outcome of a cancer-related cell detection assay to confirm the presence or absence of a metastasis and/or the presence or absence of CTCs. Any convenient method of testing a subject may be employed, including but not limited to e.g., any of those testing steps, as described above, which may be performed following a screening procedure. In some instances, a method of treating a subject may exclude further testing apart from a cancer-related cell detection assay as described herein. For example, a subject may be treated based on the outcome of a cancer-related cell detection assay, as described herein, without further confirmatory testing.

### Kits

Also provided are kits for practicing one or more of the above-described methods. The subject kits may vary greatly. Reagents and devices included in the subject kits may include those mentioned above with respect to the methods of detecting a cancer-related cell type and/or a population thereof, such as a TIL population, methods of screening a subject, methods of treating a subject, and the like.

In some embodiments, a Kit of the present disclosure may include a detectable binding member that specifically binds to an immune checkpoint marker or such a kit may include a plurality of detectable binding members that each specifically bind to an immune checkpoint marker. For example, in some instances, a kit of the present disclosure may include an immune checkpoint panel that includes a plurality of detectable immune checkpoint marker binding members.

In some embodiments, a Kit of the present disclosure may include a detectable binding member that specifically binds to an immune cell-type marker or such a kit may include a plurality of detectable binding members that each specifically bind to an immune cell-type marker. For example, in some instances, a kit of the present disclosure may include an immune cell-type panel that includes a plurality of detectable immune cell-type marker binding members.

Essentially any combinations of detectable binding members for detecting any marker combination, including e.g., those combinations described herein, may be employed. For example, in some instances, a kit of the present disclosure may include a detectable binding member that specifically binds PD-1, TIM-3, LAG-3 or CTLA-4. In some instances, a kit of the present disclosure may include multiple detectable binding members, where each of the multiple detectable binding members binds one of PD-1, TIM-3, LAG-3 or CTLA-4. In some instances, where multiple binding members to immune checkpoint markers are employed, a subject kit of the present disclosure may be said to include an immune checkpoint marker panel. Accordingly, the multiple binding members may be configured to bind the markers of the panel, thereby facilitating detection of cells expressing one or more markers of the panel.

In some instances, a kit of the present disclosure may include a detectable binding member that specifically binds CD3, CD4, CD8, CD16, CD19, CD25, CD56, CD127 and/or CCR5. In some instances, a kit of the present disclosure may include multiple detectable binding members, where each of the multiple detectable binding members binds one of CD3, CD4, CD8, CD16, CD19, CD25, CD56, CD127 and/or CCR5. In some instances, where multiple binding members to immune cell-type markers are employed, a subject kit of the present disclosure may be said to include an immune cell-type marker panel. Accordingly, the multiple binding members may be configured to bind the markers of the panel, thereby facilitating detection of cells expressing one or more markers of the panel.

The individual specific binding members of a marker panel may be provided in a pre-mixed configuration, e.g., pre-mixed in a single container such as a tube, or in individual containers to be mixed by a user as desired. In some instances, multiple components of different marker panels may be provided in a subject kit in a pre-mixed configuration, including e.g., where one or more immune checkpoint marker specific binding members or a panel thereof or a portion of such a panel may be provided pre-mixed with one or more immune cell-type marker specific binding members or a panel thereof or a portion of such a panel.

In some instances, an immune cell-type marker panel of the present disclosure may include an immune activation marker. Accordingly, a subject kit may include a detectable binding member specific for an immune activation marker, including but not limited to e.g., a detectable binding member specific for Human HLA-DR.

In some instances, a subject kit may include one or more detectable binding members specific for an adhesion-related marker. Useful detectable binding members specific for adhesion-related markers that may be included in the subject kits include but are not limited to e.g., detectable binding members specific for E cadherin, CD44 or CD103. In some instances, a kit may include a plurality of different detectable binding members each specific for a different adhesion-related marker, including e.g., two or more different detectable binding members, etc.

Kits of the present disclosure may include one or more reagents and/or devices for preparing a sample for processing and/or assaying, including e.g., for processing and/or assaying in a method as described herein. Useful reagents and/or devices that may be included in the subject kits include but are not limited to e.g., fixation reagents, fixation solution(s) that include at least one fixation reagent, a homogenization device, a device for generating a cell suspension, a device for incubating a cell suspension, a DNA labeling reagent, a device for obtaining a sample (e.g., a blood collection device, a biopsy device, an aspiration needle, etc.), and the like.

For example, in some embodiments, kits may further include one or more sample preparation reagents including but not limited to, e.g., cell fixatives, cell permeabilizing reagents, cell labeling reagents, buffers, diluents, etc. The above components may be present in separate containers or one or more components may be combined into a single container, e.g., a glass or plastic vial. In some instances, kits of the instant disclosure may further include a sample preparation device such as e.g., a homogenizer.

Kits may further include sample obtainment devices, e.g., blood collection devices or biopsy collection devices. Non-limiting examples of biopsy collection devices include but are not limited to e.g., needle biopsy devices, core biopsy devices, punch biopsy devices, surgical biopsy devices, vacuum assisted biopsy devices, etc. In some instances, kits may further include one or more reagents and/or devices for cell dissociation including but not limited to e.g., enzymes, enzyme inhibitors, detergents, cell dissociation media or buffer, vortex devices, nutating devices, rocking devices, etc. Subject kits may further include control reagents and samples including but not limited to, e.g., control cell samples (e.g., positive control cellular samples, negative control cellular samples, etc.) calibration reagents (e.g., fluorescent beads, pre-labeled cells, etc.).

In addition to the above components, the subject kits may further include instructions for practicing the subject methods. These instructions may be present in the subject kits in a variety of forms, one or more of which may be present in the kit. One form in which these instructions may be present is as printed information on a suitable medium or substrate, e.g., a piece or pieces of paper on which the information is printed, in the packaging of the kit, in a package insert, etc. Yet another means would be a computer readable medium, e.g., diskette, CD, etc., on which the information has been recorded. Yet another means that may be present is a website address which may be used via the internet to access the information at a removed site. Any convenient means may be present in the kits.

The following example(s) is/are offered by way of illustration and not by way of limitation.

### EXAMPLES

The following examples are put forth so as to provide those of ordinary skill in the art with a complete disclosure and description of how to make and use the present invention, and are not intended to limit the scope of what the inventors regard as their invention nor are they intended to represent that the experiments below are all or the only experiments performed. Efforts have been made to ensure accuracy with respect to numbers used (e.g. amounts, temperature, etc.) but some experimental errors and deviations should be accounted for. Unless indicated otherwise, parts are parts by weight, molecular weight is weight average molecular weight, temperature is in degrees Centigrade, and pressure is at or near atmospheric.

General methods in molecular and cellular biochemistry can be found in such standard textbooks as Molecular Cloning: A Laboratory Manual, 3rd Ed. (Sambrook et al., HaRBor Laboratory Press 2001); Short Protocols in Molecular Biology, 4th Ed. (Ausubel et al. eds., John Wiley & Sons 1999); Protein Methods (Bollag et al., John Wiley & Sons 1996); Nonviral Vectors for Gene Therapy (Wagner et al. eds., Academic Press 1999); Viral Vectors (Kaplift & Loewy eds., Academic Press 1995); Immunology Methods Manual (I. Lefkovits ed., Academic Press 1997); and Cell and Tissue Culture: Laboratory Procedures in Biotechnology (Doyle & Griffiths, John Wiley & Sons 1998), the disclosures of which are incorporated herein by reference. Reagents, cloning vectors, and kits for genetic manipulation referred to in this disclosure are available from commercial vendors such as BioRad, Stratagene (Agilent Technologies), Invitrogen (Thermo Fisher Scientific), Sigma-Aldrich, and Clontech (Takara Bio USA, Inc.).

### Example 1: Cell by Cell Immuno- and Cancer Marker Profiling of Non-Small Cell Lung Cancer Tissue Sample Reveals Correlations of Immune Check Point and Cancer Marker Expressions with Metastasis

### Materials and Methods

### Samples

Fresh tissues were obtained from 10 NSCLC cases by Folio Biosciences (Powell, Ohio) with paired peripheral blood sample following informed consent. Date of collection, age, sex, ethnicity, diagnosis, primary tumor size, and American Joint Committee on Cancer (AJCC) classification were recorded. Tissues were excised and then stored in Roswell Park Memorial Institute (RPMI) 1640 medium at 2-8° C. prior to overnight shipment to IncellDx Inc. on cold packs. The blood samples from the same patients were shipped to and processed at Celsee Inc. (Plymouth, Mich.).

### Tissue Dissociation Using IncellPREP

Tumor biopsies of at least 2 cm were placed in RPMI for transport after which 4-mm punches were taken from each tissue, and placed in 2-mL Eppendorf tubes containing 800 μL Dulbecco's phosphate-buffered saline (DPBS). IncellPREP (IncellDx, Inc.) tissue homogenizers were inserted into each tube and set to run at 1 V until supernatant appeared cloudy (5-10 min)′. After tissue homogenization, cells were counted using a hemocytometer to calculate cellular concentration, and cell counts were recorded. As the next step, the supernatant was then removed and transferred to a separate tube before centrifugation at 300×g for 5 min. Following centrifugation, the supernatant was aspirated, and cell pellet was resuspended in IncellMax reagent at a concentration of 1×10⁶ cells/mL and incubated at room temperature for 1 hour prior to staining. The remaining samples were cryopreserved at −80° C. for future use.

### Immune Check Point Panel and Cancer Marker Panel Staining of Single Cell Suspensions

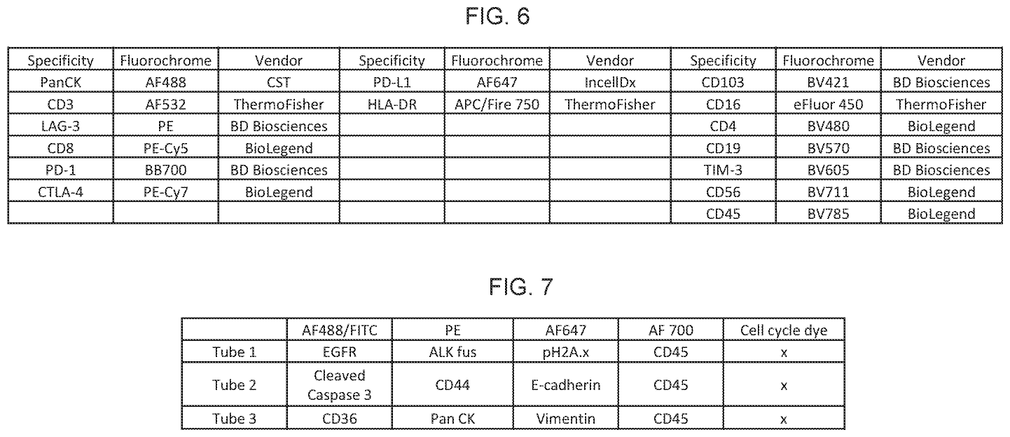

Following fixation for 1 hour in IncellMAX (also known as IncellFP; IncellDx, Inc.), 500 μL of sample equivalent to 500,000 cells was aliquoted to 12×75 mm tubes and subsequently washed with 1 mL of DPBS. Samples were then stained with the immune panel (shown in Table 1, provided in FIG. 6) in DPBS+2% bovine serum albumin (BSA) and then incubated for 30 min at room temperature in the dark. Next, 1 mL of DPBS+2% BSA was added to each tube and incubated at room temperature for 5 min, prior to centrifugation at 600×g for 5 min. The supernatant was aspirated, and a wash with DPBS+2% BSA was repeated once. Following this wash, 300 μL of DPBS was added to each sample. (CST: Cell Signaling Inc., Danvers, Mass.)

For cancer marker panels, 200,000 cells were stained for each tube following the same procedure listed above with the additional step of adding 200 μL of 4′,6-diamidino-2-phenylindole (DAPI) at 1 μg/mL concentration in DPBS to each sample and followed by incubation at room temperature for 30 minutes in the dark. The cancer marker panels are shown in Table 2, provided in FIG. 7. All antibodies used were from Cell Signaling Inc., except for CD36 (BioLegend Inc.). The cell cycle dye was supplied from IncellDx.

### Flow Cytometry

The cells stained with the 19-color immune marker panel were analyzed on a Cytek Aurora™ cytometer equipped with 3 lasers (Cytek Biosciences Inc, Fremont, Calif.). The cells stained with the 5-color cancer marker panels were analyzed on a 3-laser CytoFlex™ cytometer (Beckman Coulter, Miami, Fla.). The FCS files collected by Aurora were analyzed by SpectroFlo™ software. FCS files collected from CytoFlex were analyzed with CytExpert™ software.

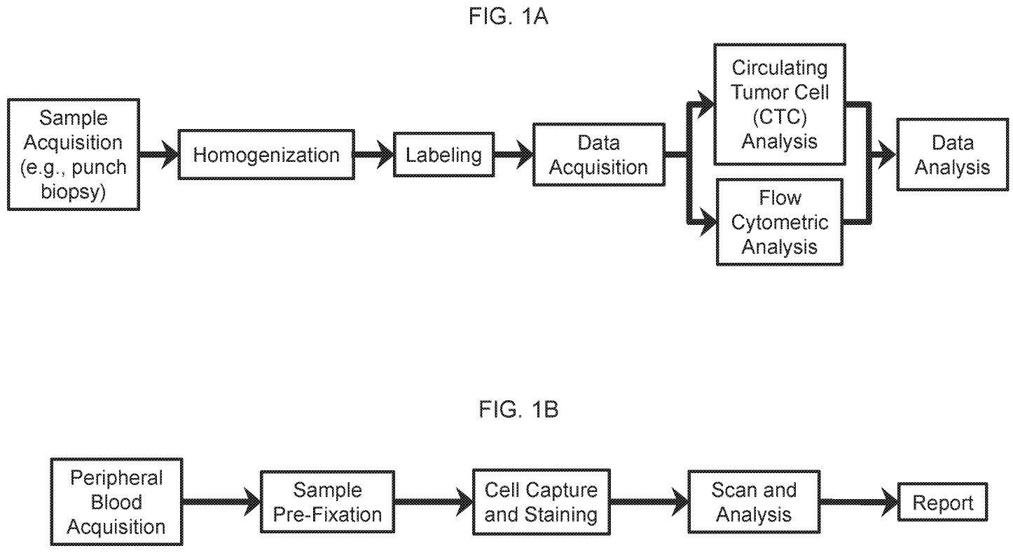

The workflow for solid tumor tissue processing and analysis is shown in FIG. 1A.

### DNA Index Calculation

DNA index was calculated as cancer cell DAPI MFI (median fluorescent intensity) divided by diploid cell (lymphocytes in this study) MFI.

### CTC Isolation and Enumeration

Patient peripheral blood samples were received in a Streck cfDNA BCT and kept at ambient temperature. 4 mL of the blood samples was prefixed using Celsee prefixation buffer. Each sample was then processed utilizing the Celsee Comprehensive Single Cell Preparation chip, Genesis automated platform and enumeration protocol. Upon completion, the chips were scanned on the Celsee Analyzer where a final report was generated. For each individual cell detection of CD45, PD-L1, and DNA content (DAPI) markers were assessed.

The workflow for CTC isolation and analysis is shown in FIG. 1B.

### Statistical Analysis

All sample data collected were compared against CTC. Statistical significance was determined using Spearman analysis.

### Results

### Cancer Marker Expression

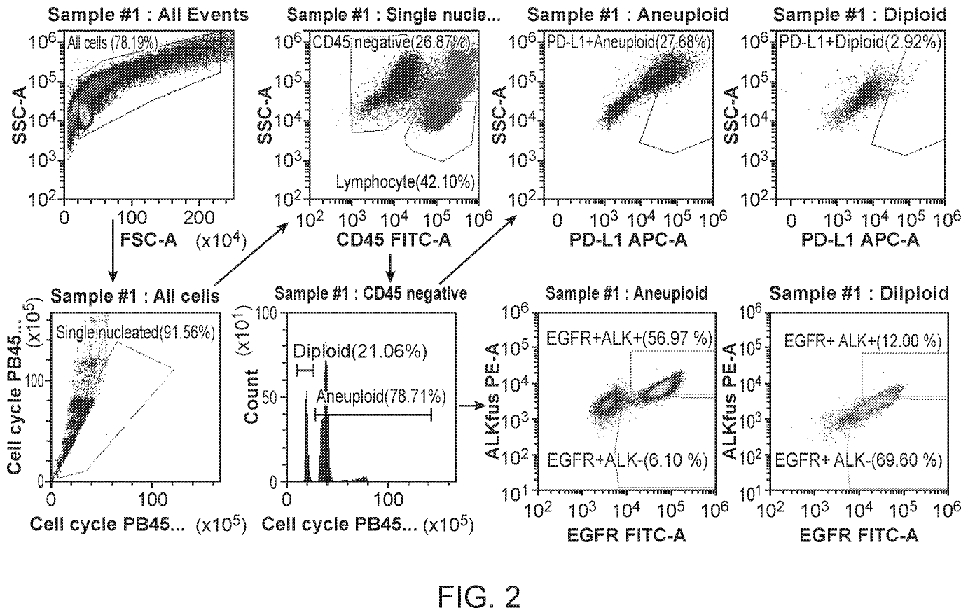

Single nucleated cells were gated using light scattering and DAPI staining (see FIG. 2). DAPI staining was also employed to separate cells with high DNA content (aneuploid cells) from cells with normal DNA content (diploid cells). The “Aneuploid” gate includes cells having higher DNA content due to either chromosomal level mutation resulting in increased chromosomes or due to increased cell proliferation. DAPI staining also provided the MFI values for DNA index calculation. CD45 serves as a marker of immune cells. Cancer cells are CD45− and Pan CK+ epithelial cells. Mesenchymal or stromal cells are CD45− and vimentin+. Cleaved caspase 3 is a common apoptotic pathway for both death receptors and mitochondrial cell death leading to the finding that a cell undergoing active apoptosis are positive for cleaved caspase 3. The histone pH2A.x (ser 139) is considered a marker for DNA damaged. Cells with damaged DNA were stained positive for pH2A.x. Overexpression of total EGFR is a lung cancer phenotype with greater than 80% NSCLC exhibiting EGFR overexpression. The ALK fusion protein is a target for specific therapy. E-cadherin is an alpha integrin known for cell-cell adhesion, which is, in some instances, lost in CTCs. CD44 is highly expressed on cancer stem cells (CSCs) with diploid DNA content. CD36 is a fat molecule that is thought to be involved in energy transfer necessary for metastasis.

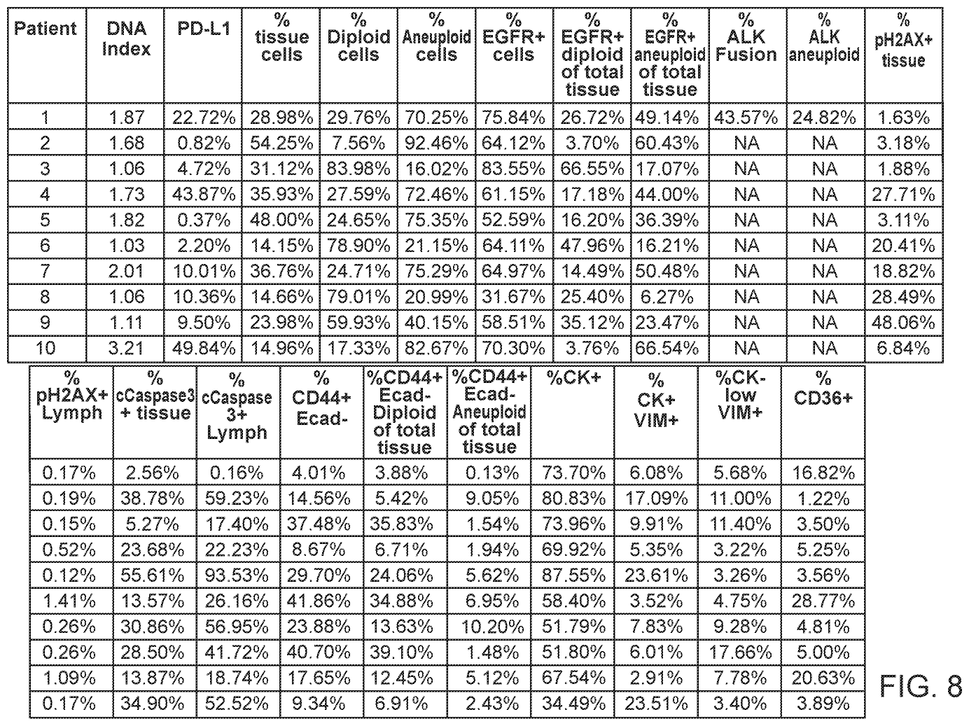

The results for the 10 NSCLC cancer samples are summarized in Table 3, provided in FIG. 8.

EGFR overexpression was observed in all the cancer samples investigated. The overexpression was observed in both diploid an aneuploidy cells, suggesting either genetic alterations of EGFR or other signaling pathways might have contributed to the EGFR overexpression. ALK mutation was observed in one cancer sample, which is consistent with other observations that 3-7% of NSCSC samples harbor the ALK mutation. The ALK mutated cells also overexpressed EGFR and also had higher DNA contents (aneuploid cells). This observation suggests the presence of other genetic alterations that occurred simultaneously with ALK fusion which were previously overlooked.

Lymphocytes did exhibit DNA damage, suggesting lymphocytes are not directly a part of the cancer forming process. In stark contrast, a significant percentage of the lymphocytes were undergoing apoptosis as shown by the cleaved caspase 3 staining. This is consistent with immune suppressive mechanisms utilized by cancer that would have caused death of the lymphocytes. The cancer tissue showed higher levels of DNA damage compared with lymphocytes, as evidenced by positive pH2A.x staining. In most cases, fewer cancer cells underwent apoptosis as compared with lymphocytes.

The majority of the cancer cells are epithelial cells (pan CK+). A smaller portion of the tissue cells are mesenchymal/stromal cells (vimentin+). In these cancer samples, some cells were pan CK and vimentin double positive suggesting a possible link to the epithelial and mesenchymal cell transition². The mesenchymal cells are diploid cells. The double positive cells have ploidy similar to the primary cancer epithelial cells (pan CK+).

In the same samples, a distribution of CD44+E-cad− cells with diploid DNA content were present at various levels. CD44 may serve a cancer stem cell marker in this context. E-cadherin plays an important role in epithelial cell adhesion and the loss of E-cadherin function is considered to be a major contributor to epithelial mesenchymal transition (EMT)³⁻⁴. Cells with CD44+E-cad− phenotype have been reported to be cancer stem cells.

All samples showed various low percentages of CD36+ cancer cells. These fat cells might provide energy for tumor metastasis.

### Immune Check Point Marker Expressions in the Tumor Micro-Environment

The immune system plays a dual role in cancer development. It does not only suppress cancer by destroying cancer cells or inhibiting cancer growth, but also promotes cancer growth by selecting the cancer cells that can escape immune surveillance⁵⁻⁶. Turning on the immune check-point marker pathway is a mechanism cancer cells utilize to escape immune suppression. An in depth look at immune check-point marker expression in a cancer microenvironment provides us with valuable information. Many studies have been conducted by RNA expression studies on bulkily processed cells, which does not provide functional protein information.

In this study, spectral high-parameter flow cytometry was applied using a 3-laser Cytek Aurora, which allowed for interrogation of a total of 14 markers simultaneously. In one tube, cancer single cell suspension samples were stained with markers that facilitate gating to separate cancer immune cell subsets, immune check point markers, along with HLA-DR, an immune activation marker, and CD103, a marker indicating the engagement of immune cells to cancer.

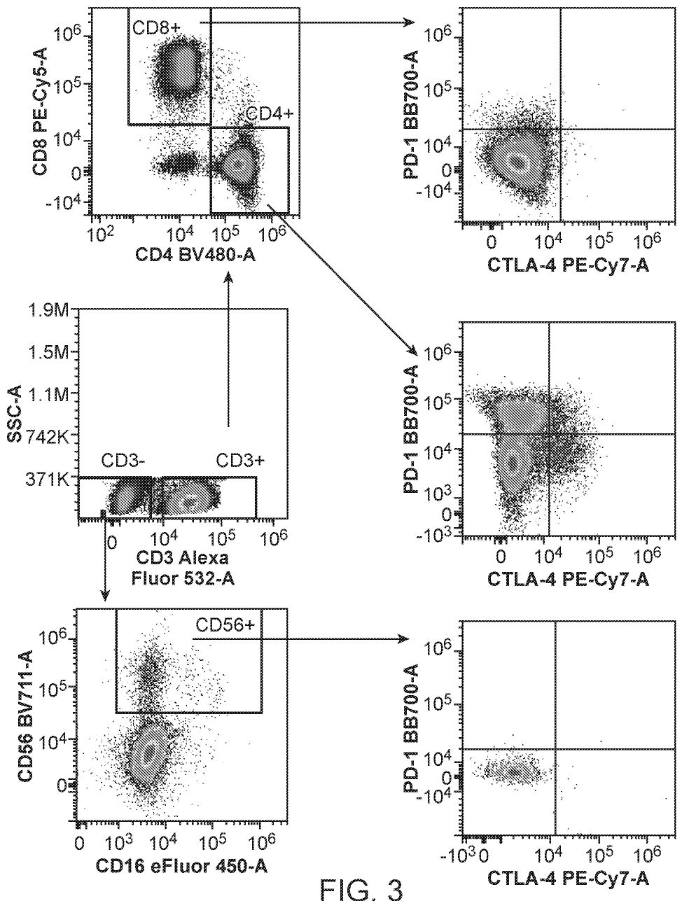

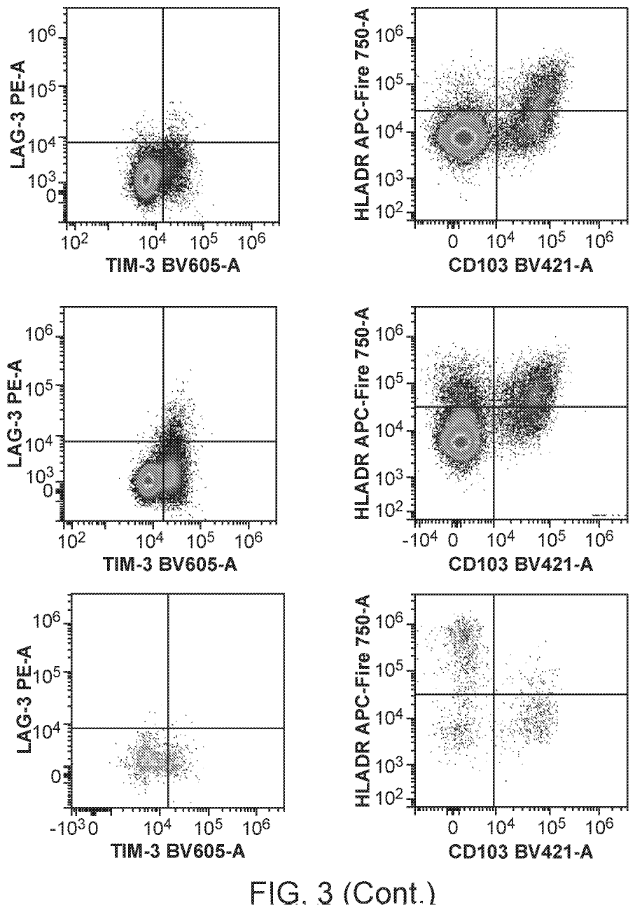

Forty to 350 thousand single nucleated cells were acquired from each of the 10 samples. The cells were separated into immune cell subsets by gating markers. Immune check point markers, HLA-DR and CD103 marker expression were interrogated (FIG. 3).

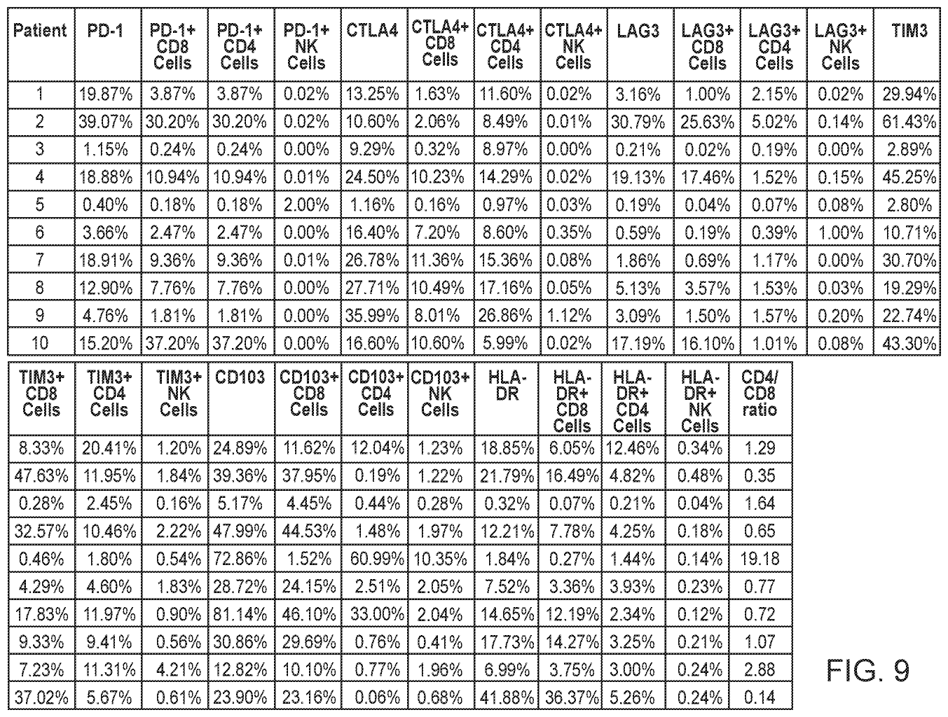

Very few B cells were detected in the cancer microenvironment. Various percentages of CD4+, CD8+ T cells, and a low percentage of NK cells were observed in the ten NSCLC samples. PD-1, CTLA-4, LAG-3, TIM-3, HLA-DR, and CD103 showed differential expression on these cell subpopulations. The data is summarized in Table 4, provided in FIG. 9.

### CTC Isolation and Analysis

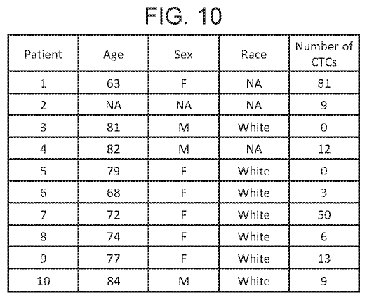

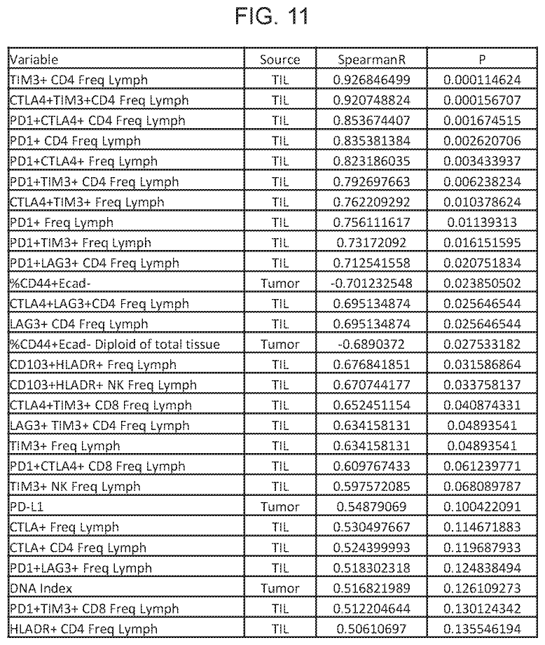

In parallel to analyzing the cancer tissue samples, peripheral blood samples were shipped to Celsee Inc. for CTC analysis. CTCs were isolated as described in the Materials and Methods. The CTCs isolated were stained with CD45, PD-L1 and DAPI for characterization. The CTC numbers for each cancer patient are summarized in Table 5, provided in FIG. 10.

### TIM-3 and PD-1 Expressing CD4+ T Cells Strongly Correlates to CTCs

In order to better understand the relationships between cancer and immune marker expression with potential cancer metastasis, a statistical analysis was performed on the collected flow data. Since there were 10 NSCLC samples, a Spearman correlation analysis was used for data analysis, measuring rank correlation between the rankings of two variables rather than the absolute values. The percentage of cells expressing each cancer marker and immune marker were compared against CTC numbers. A number of dual marker expressing cell populations were also compared against CTCs.

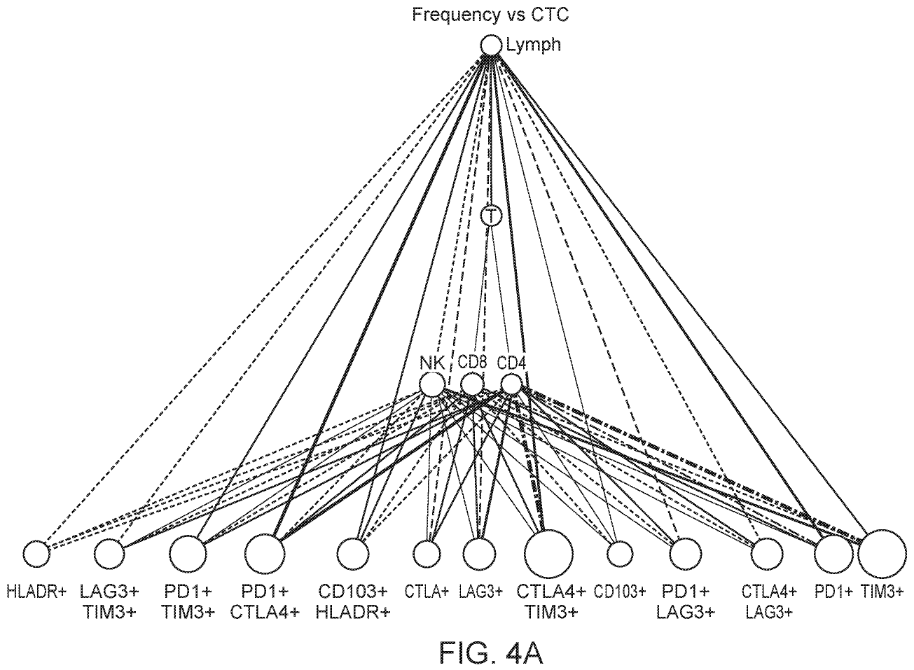

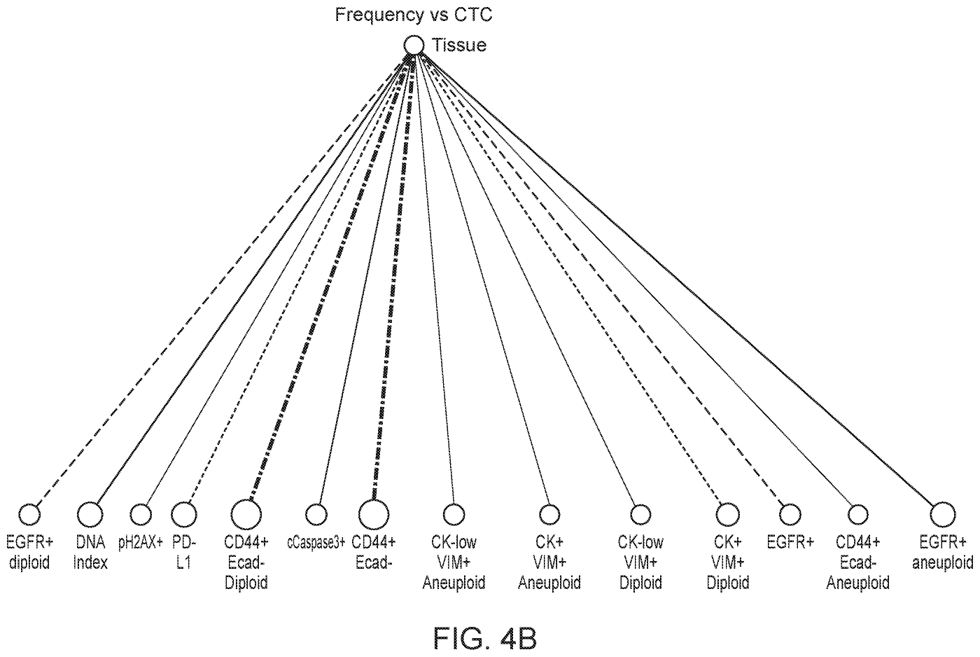

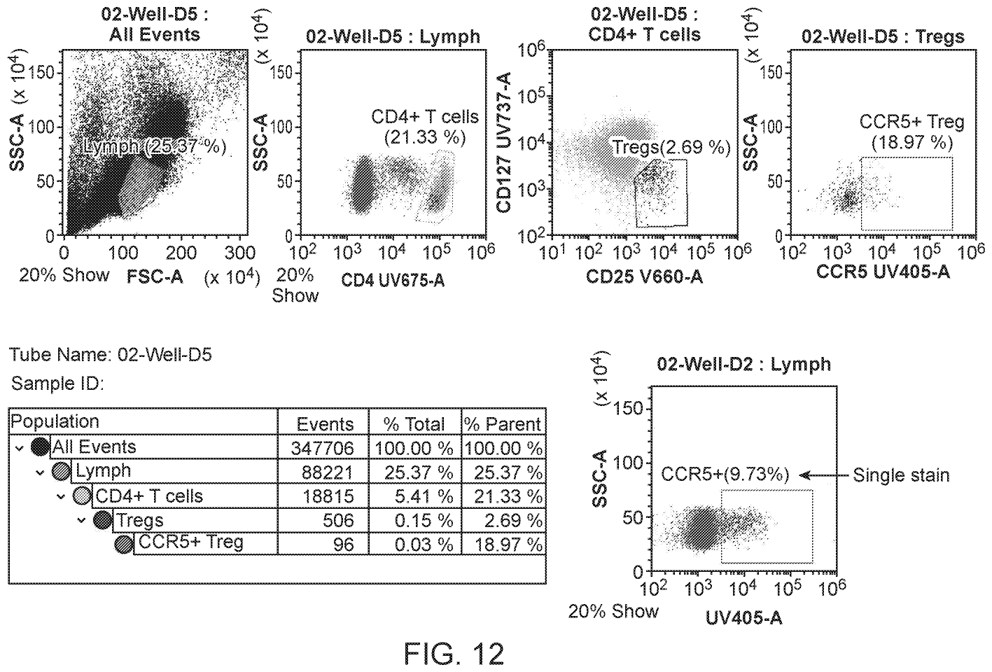

Spearman analysis revealed, e.g., aa strong correlation between CTC and TIM-3 and PD-1 expressing lymphocytes, especially CD4+ lymphocytes (FIG. 4A and a Supplemental Table showing Spearman R absolute values above 0.5, provided as FIG. 11).

A correlation was also observed between CTC and CD103 and HLA-DR double positive lymphocytes. The CD44+Ecad− cancer stem cells reversely correlated to CTCs (FIG. 4B and a Supplemental Table showing Spearman R absolute values above 0.5, provided as FIG. 11). PD-L1 expressing cancer cells and cancer cell DNA content also displayed a correlation with CTCs.

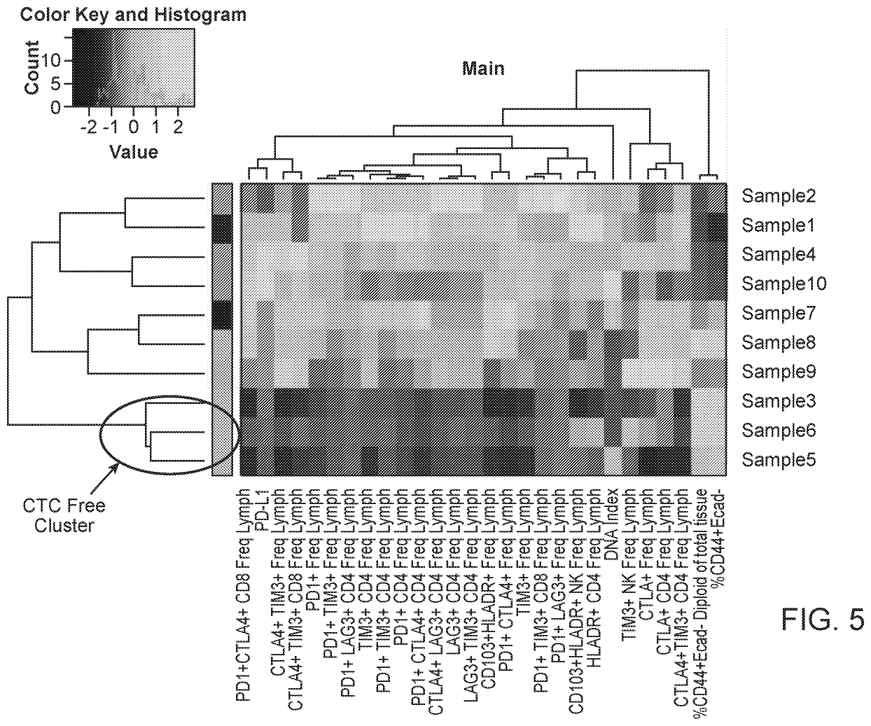

Unsupervised Spearman clustering analysis clustered patients with low or no CTC (Patients #3, #6, #5) and separated these patients from patients with high CTC numbers (FIG. 5).

### Discussion

Metastasis accounts for 90% of all cancer death. The complete mechanism causing metastasis is still largely unknown⁷. Due to the limitations of established technologies, investigators either perform IHC to study a limited number cancer markers or immune cell markers, or study bulk lysed cancer tissue samples for RNA expression en masse.

Here NSCLC solid cancer tissues were dissociated into single cells and the cells were stained with cancer and immune markers. The dissociated and stained cells were analyzed with high-parametric flow cytometry. This workflow allowed for the study of multiple immune markers and cancer markers at the same time. In parallel, peripheral blood samples from the same patients were also collected to isolate, enumerate and analyze the CTCs as an indicator for cancer metastasis.

In contrast to earlier work to study the contributions of either cancer markers or immune markers in cancer metastasis, this study was designed to study both the cancer and the immune response in the cancer microenvironment. Statistical data analysis revealed correlations between cancer marker and immune marker expression with the prevalence of CTCs.

Our results showed immune check-point markers, e.g., TIM-3 and PD-1, accounted for significant contributions to CTCs. Interestingly, the expression of these markers on CD4+ cells correlated more strongly as compared to the normally recognized CD8+ cytotoxic T cells⁸.

In addition, the expression of CD103 and HLA-DR correlated strongly with CTCs. This suggests the active engagement of the immune system to fight cancer in spite of the strong immune blocking activities of immune check point molecules. This observation suggests that cancer activation might be accounted for by a breakage in the balance between immune blocking and immune activation.

DNA instability (as indicated by DNA index) and PD-L1 expressions on cancer cells showed significant correlation with cancer metastasis. In stark contrast, the cancer stem cells (CD44+E-cad-) significantly reversely correlated with CTC prevalence, suggesting the presence of a high number of quiescent cancer stem cells in the same mass.

Moreover, the unsupervised Spearman clustering analysis separated patients with high CTCs from those with low CTCs. This shows that the algorithm generated from multi-parametric flow data can provide predictive, actionable information to patient management.

Multi-parametric flow cytometry pairing with single cell dissociation allowed simultaneous profiling of multiple immune and cancer markers on solid tumor samples at the single cell level. The knowledge acquired from these studies will enhance our understanding of the cancer immune system, cancer cells, and the interaction between the immune system and the cancer. Such information can transform patient diagnosis, disease monitoring, and drug discovery. This study demonstrates a powerful method to study solid tumors that will provide important information for successful therapy, such as precision cancer immunotherapy.

### Example 2: Detection of CCR5 Expressing Treg Cells in Lung Tumor Tissue

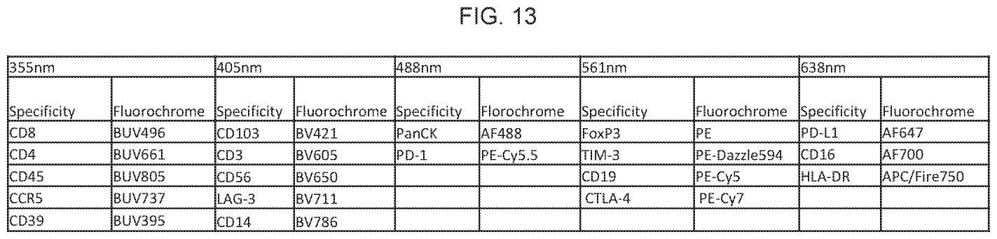

Frozen samples of lung tumor tissue were thawed, dissociated, and stained with a marker panel for multi-parameter flow cytometric analysis essentially as described above. The marker panel employed included detectable binding members for CD4, CD25, CD103, CD127 and CCR5 (CD195). An example of a marker panel that may be employed for multi-parametric analysis to interrogate CCR5 expression in tumor-associated immune cells is provided in FIG. 13.

Stained cell suspension was flow cytometrically analyzed using the multi-parameter flow cytometric techniques described above. Events were gated for Treg as depicted in FIG. 12. Specifically, CD4 positive T cells were differentiated based CD4 staining and on forward scatter and side scatter gates for lymphocytes. Tregs were identified within the CD4⁺ T cell population based on high CD25 staining and low CD127 staining. Next CCR5 staining in the identified Treg population was evaluated and CCR5⁺ Treg cells were detected. For comparison to the multi-parameter assay employed, single-parameter analysis (i.e., “single staining”) for CCR5 lymphocytes is also shown in FIG. 12. These results demonstrate the detection of CCR5 positive Treg cells using multi-parameter analysis of lung tumor tissue stained using a marker panel as described herein. Accordingly, in this example, the employed methods readily detect a cell population, namely CCR5⁺ immune cells, in a tumor sample from a subject that is important for assessing tumor spread and metastasis in the subject.

### REFERENCES

1. Chargin A et al. Quantification of PD-L1 and PD-1 expression on tumor and immune cells in non-small cell lung cancer (NSCLC) using non-enzymatic tissue dissociation and flow cytometry. Cancer immunol immunother. 65(11):1317-1323. 2016
2. Gonzalez et al. Commonly Occurring Cell Subsets in High-Grade Serous Ovarian Tumors Identified by Single-Cell Mass Cytometry. Cell report 22, 1875-1888. 2018
3. Nieto M A, et al. EMT: 2016. Cell. 166: 21. 2016
4. Heerboth S, et al. EMT and tumor metastasis. Clinical and Translational Medicine. 4:6, 2015
5. Schreiber D S et al. Cancer immunoediting: integrating immunity's roles in cancer suppression and promotion. Science. 331: 1565. 2011
6. Mittal D et al. New insights into cancer immunoediting and its three component phases—elimination, equilibrium and escape. Curr Opin Immunol. 27: 16. 2014
7. Seyfriend T N et al. On the origin of cancer metastasis. Crit rev oncog. 18(1-2):43-73. 2013
8. Yi J S et al. T-cell exhaustion: characteristics, causes and conversion. Immunology. 129:474-481. 2010

Notwithstanding the appended claims, the disclosure is also defined by the following clauses:

1. A method comprising:

contacting a cellular suspension prepared from a biopsy collected from a tumor in a subject with:

an immune checkpoint panel comprising a plurality of detectable immune checkpoint marker binding members; and

an immune cell-type panel comprising a plurality of detectable immune cell-type marker binding members,

to produce a labeled cell suspension; and

cytometrically assaying the labeled cell suspension to detect whether a tumor infiltrating lymphocyte (TIL) population expressing an immune checkpoint marker of the immune checkpoint panel and an immune cell-type marker of the immune cell-type panel is present in the cellular suspension.

2. The method according to Clause 1, wherein the plurality of detectable immune cell-type marker binding members comprises specific binding members for two or more immune checkpoint markers selected from the group consisting of: programmed cell death 1 (PD-1), T-cell immunoglobulin mucin receptor 3 (TIM-3), lymphocyte-activation gene-3 (LAG-3) and cytotoxic T-lymphocyte associated protein 4 (CTLA-4).
3. The method according to Clauses 1 or 2, wherein the plurality of detectable immune cell-type marker binding members comprises specific binding members for two or more immune cell-type markers selected from the group consisting of CD3, CD4, CD8, CD16, CD19, CD25, CD56, CD127 and CCR5.
4. The method according to any of the preceding clauses, wherein the immune checkpoint marker expressed by the TIL population is PD-1.
5. The method according to Clause 4, wherein the TIL population expresses a combination of markers selected from the group consisting of:

PD-1 and CTLA-4;

PD-1, CTLA-4 and CD4;

PD-1, CTLA-4 and CD8;

PD-1 and CD4;

PD-1 and TIM-3;

PD-1, TIM-3 and CD4; and

PD-1, LAG-3 and CD4.

6. The method according to any of Clauses 1 to 3, wherein the immune checkpoint marker expressed by the TIL population is TIM-3.
7. The method according to Clause 6, wherein the TIL population expresses a combination of markers selected from the group consisting of:

TIM-3 and CD4;

TIM-3, CD4 and LAG3;

TIM-3 and CTLA-4;

TIM-3, CTLA-4 and CD4;

TIM-3, CTLA-4 and CD8; and

TIM-3 and an NK cell marker.

8. The method according to any of Clauses 1 to 3, wherein the TIL population expresses a combination of LAG-3 and CD4.
9. The method according to Clause 8, wherein the TIL population expresses a combination of LAG-3, CD4 and CTLA-4.
10. The method according to any of the preceding clauses, wherein the method comprises cytometrically assaying the cellular suspension to determine whether two or more different TIL populations are present in the cellular suspension.
11. The method according to any of the preceding clauses, wherein the immune cell-type panel further comprises a detectable binding member specific for an immune activation marker.
12. The method according to Clause 11, wherein the immune activation marker is Human Leukocyte Antigen-antigen D Related (HLA-DR).
13. The method according to any of the preceding clauses, wherein the cellular suspension is further contacted with a detectable binding member specific for an adhesion-related marker.
14. The method according to Clause 13, wherein the adhesion-related marker is selected from the group consisting of: E cadherin, CD44 and CD103.
15. The method according to Clauses 13 or 14, wherein the cellular suspension is contacted with a plurality of detectable binding members each specific for an adhesion-related marker.
16. The method according to any of the preceding clauses, wherein the method comprises cytometrically assaying for the presence or absence of a further TIL population that is E cadherin negative and expresses CD44.
17. The method according to any of the preceding clauses, wherein the method comprises cytometrically assaying the cellular suspension to further determine whether a TIL population that expresses CD103 and HLA-DR is present in the cellular suspension.
18. The method according to Clause 17, wherein the further TIL population expresses CD103, HLA-DR and a NK cell marker.
19. The method according to any of the preceding clauses, wherein the method further comprises dissociating the biopsy, or a sample therefrom, to prepare the cellular suspension.
20. The method according to Clause 19, wherein the dissociating comprises non-enzymatic tissue homogenization.
21. The method according to any of the preceding clauses, wherein the cellular suspension is further contacted with a fixation reagent.
22. The method according to any of the preceding clauses, wherein the biopsy is a solid tissue biopsy.
23. The method according to any of the preceding clauses, wherein the tumor is a lung cancer tumor.
24. The method according to Clause 23, wherein the lung cancer tumor is a non-small cell lung cancer (NSCLC) tumor.
25. The method according to any of the preceding clauses, wherein the cellular suspension is further contacted with a DNA staining reagent.
26. The method according to Clause 25, wherein the cytometrically assaying further comprises quantifying per cell DNA content based on the DNA staining reagent.
27. The method according to any of the preceding clauses, wherein the method further comprises contacting the cellular suspension with at least one detectable binding member specific for a mesenchymal cell marker or epithelial cell marker.
28. The method according to Clause 27, wherein the at least one detectable binding member specific for a mesenchymal cell marker or epithelial cell marker comprises: a detectable binding member specific for Pan CK; a detectable binding member specific for vimentin; or both.
29. A kit comprising:

an immune checkpoint panel comprising a plurality of detectable immune checkpoint marker binding members; and

an immune cell-type panel comprising a plurality of detectable immune cell-type marker binding members.

30. The kit according to Clause 29, wherein the plurality of detectable immune cell-type marker binding members comprises specific binding members for two or more immune checkpoint markers selected from the group consisting of: PD-1, TIM-3, LAG-3 and CTLA-4.
31. The kit according to Clauses 29 or 30, wherein the plurality of detectable immune cell-type marker binding members comprises specific binding members for two or more immune checkpoint markers selected from the group consisting of CD3, CD4, CD8, CD16, CD19, CD25, CD56, CD127 and CCR5.
32. The kit according to any of Clauses 29 to 31, wherein the immune cell-type panel further comprises a detectable binding member specific for an immune activation marker.
33. The kit according to Clause 32, wherein the immune activation marker is Human HLA-DR.
34. The kit according to any of Clauses 29 to 33, further comprising a detectable binding member specific for an adhesion-related marker.
35. The kit according to Clause 34, wherein the adhesion-related marker is selected from the group consisting of: E cadherin, CD44 and CD103.
36. The kit according to Clauses 34 or 35, wherein the kit comprises a plurality of detectable binding members each specific for an adhesion-related marker.
37. The kit according to any of Clauses 29 to 36, further comprising a cell suspension fixation solution comprising a fixation reagent.
38. The kit according to any of Clauses 29 to 37, further comprising a homogenization device.
39. The kit according to any of Clauses 29 to 38, further comprising a DNA labeling reagent.
40. The kit according to any of Clauses 29 to 39, further comprising a biopsy collection device.
41. A method of screening a subject for metastatic cancer, the method comprising:

detecting the presence of the TIL population according to the method of any of Clauses 1 to 28; and

testing the subject for a metastatic cancer based on the detected presence of the TIL population.

42. The method according to Clause 41, wherein the testing comprises collecting a sample from the subject.
43. The method according to Clause 42, wherein the sample is a liquid sample comprising blood or lymph.
44. The method according to Clause 43, wherein the liquid sample is subjected to a circulating tumor cell (CTC) assay.
45. The method according to Clause 44, wherein the CTC assay is a microfluidic CTC assay.
46. The method according to any of Clauses 41 to 45, wherein the testing comprises a lymph node biopsy.
47. The method according to any of Clauses 41 to 46, wherein the testing comprises medical imaging.
48. A method of treating a subject for a neoplasia, the method comprising:
detecting the presence or absence of the TIL population according to the method of any of Clauses 1 to 28; and

treating the subject for the neoplasia, wherein the treating comprises:

a systemic treatment for the neoplasia when the TIL population is detected to be present; or

a local treatment for the neoplasia when the TIL is detected to be absent.

49. The method according to Clause 48, wherein the method further comprises testing the subject to confirm the presence or absence of metastasis.
50. The method according to Clause 49, wherein the testing comprises confirming the presence or absence of CTCs in a sample collected from the subject.
51. The method according to any of Clauses 48 to 50, wherein the systemic treatment for the neoplasia comprises systemic radiation therapy, chemotherapy, immunotherapy or a combination thereof.
52. The method according to any of Clauses 48 to 51, wherein the local treatment for the neoplasia comprises surgery, local radiation therapy, cryotherapy, laser therapy, topical therapy or a combination thereof.
53. The method according to any of Clauses 48 to 52, wherein the treating excludes one or more systemic treatments when the TIL population is absent.

In at least some of the previously described embodiments, one or more elements used in an embodiment can interchangeably be used in another embodiment unless such a replacement is not technically feasible. It will be appreciated by those skilled in the art that various other omissions, additions and modifications may be made to the methods and structures described above without departing from the scope of the claimed subject matter. All such modifications and changes are intended to fall within the scope of the subject matter, as defined by the appended claims.

It will be understood by those within the art that, in general, terms used herein, and especially in the appended claims (e.g., bodies of the appended claims) are generally intended as “open” terms (e.g., the term “including” should be interpreted as “including but not limited to,” the term “having” should be interpreted as “having at least,” the term “includes” should be interpreted as “includes but is not limited to,” etc.). It will be further understood by those within the art that if a specific number of an introduced claim recitation is intended, such an intent will be explicitly recited in the claim, and in the absence of such recitation no such intent is present. For example, as an aid to understanding, the following appended claims may contain usage of the introductory phrases “at least one” and “one or more” to introduce claim recitations. However, the use of such phrases should not be construed to imply that the introduction of a claim recitation by the indefinite articles “a” or “an” limits any particular claim containing such introduced claim recitation to embodiments containing only one such recitation, even when the same claim includes the introductory phrases “one or more” or “at least one” and indefinite articles such as “a” or “an” (e.g., “a” and/or “an” should be interpreted to mean “at least one” or “one or more”); the same holds true for the use of definite articles used to introduce claim recitations. In addition, even if a specific number of an introduced claim recitation is explicitly recited, those skilled in the art will recognize that such recitation should be interpreted to mean at least the recited number (e.g., the bare recitation of “two recitations,” without other modifiers, means at least two recitations, or two or more recitations). Furthermore, in those instances where a convention analogous to “at least one of A, B, and C, etc.” is used, in general such a construction is intended in the sense one having skill in the art would understand the convention (e.g., “a system having at least one of A, B, and C” would include but not be limited to systems that have A alone, B alone, C alone, A and B together, A and C together, B and C together, and/or A, B, and C together, etc.). In those instances where a convention analogous to “at least one of A, B, or C, etc.” is used, in general such a construction is intended in the sense one having skill in the art would understand the convention (e.g., “a system having at least one of A, B, or C” would include but not be limited to systems that have A alone, B alone, C alone, A and B together, A and C together, B and C together, and/or A, B, and C together, etc.). It will be further understood by those within the art that virtually any disjunctive word and/or phrase presenting two or more alternative terms, whether in the description, claims, or drawings, should be understood to contemplate the possibilities of including one of the terms, either of the terms, or both terms. For example, the phrase “A or B” will be understood to include the possibilities of “A” or “B” or “A and B.”

In addition, where features or aspects of the disclosure are described in terms of Markush groups, those skilled in the art will recognize that the disclosure is also thereby described in terms of any individual member or subgroup of members of the Markush group.

As will be understood by one skilled in the art, for any and all purposes, such as in terms of providing a written description, all ranges disclosed herein also encompass any and all possible sub-ranges and combinations of sub-ranges thereof. Any listed range can be easily recognized as sufficiently describing and enabling the same range being broken down into at least equal halves, thirds, quarters, fifths, tenths, etc. As a non-limiting example, each range discussed herein can be readily broken down into a lower third, middle third and upper third, etc. As will also be understood by one skilled in the art all language such as “up to,” “at least,” “greater than,” “less than,” and the like include the number recited and refer to ranges which can be subsequently broken down into sub-ranges as discussed above. Finally, as will be understood by one skilled in the art, a range includes each individual member. Thus, for example, a group having 1-3 articles refers to groups having 1, 2, or 3 articles. Similarly, a group having 1-5 articles refers to groups having 1, 2, 3, 4, or 5 articles, and so forth.

Although the foregoing invention has been described in some detail by way of illustration and example for purposes of clarity of understanding, it is readily apparent to those of ordinary skill in the art in light of the teachings of this invention that certain changes and modifications may be made thereto without departing from the spirit or scope of the appended claims.

Accordingly, the preceding merely illustrates the principles of the invention. It will be appreciated that those skilled in the art will be able to devise various arrangements which, although not explicitly described or shown herein, embody the principles of the invention and are included within its spirit and scope. Furthermore, all examples and conditional language recited herein are principally intended to aid the reader in understanding the principles of the invention and the concepts contributed by the inventors to furthering the art, and are to be construed as being without limitation to such specifically recited examples and conditions. Moreover, all statements herein reciting principles, aspects, and embodiments of the invention as well as specific examples thereof, are intended to encompass both structural and functional equivalents thereof. Additionally, it is intended that such equivalents include both currently known equivalents and equivalents developed in the future, i.e., any elements developed that perform the same function, regardless of structure. Moreover, nothing disclosed herein is intended to be dedicated to the public regardless of whether such disclosure is explicitly recited in the claims.

The scope of the present invention, therefore, is not intended to be limited to the exemplary embodiments shown and described herein. Rather, the scope and spirit of present invention is embodied by the appended claims. In the claims, 35 U.S.C. § 112(f) or 35 U.S.C. § 112(6) is expressly defined as being invoked for a limitation in the claim only when the exact phrase “means for” or the exact phrase “step for” is recited at the beginning of such limitation in the claim; if such exact phrase is not used in a limitation in the claim, then 35 U.S.C. § 112 (f) or 35 U.S.C. § 112(6) is not invoked.

1. A method comprising:
contacting a cellular suspension prepared from a biopsy collected from a tumor in a subject with:
an immune checkpoint panel comprising a plurality of detectable immune checkpoint marker binding members; and
an immune cell-type panel comprising a plurality of detectable immune cell-type marker binding members,
to produce a labeled cell suspension; and
cytometrically assaying the labeled cell suspension to detect whether a tumor infiltrating lymphocyte (TIL) population expressing an immune checkpoint marker of the immune checkpoint panel and an immune cell-type marker of the immune cell-type panel is present in the cellular suspension.

2. The method according to claim 1, wherein the plurality of detectable immune cell-type marker binding members comprises specific binding members for two or more immune checkpoint markers selected from the group consisting of: programmed cell death 1 (PD-1), T-cell immunoglobulin mucin receptor 3 (TIM-3), lymphocyte-activation gene-3 (LAG-3) and cytotoxic T-lymphocyte associated protein 4 (CTLA-4).

3. The method according to claim 1, wherein the plurality of detectable immune cell-type marker binding members comprises specific binding members for two or more immune cell-type markers selected from the group consisting of CD3, CD4, CD8, CD16, CD19, CD25, CD56, CD127 and CCR5.

4. The method according to claim 1, wherein the method comprises cytometrically assaying the cellular suspension to determine whether two or more different TIL populations are present in the cellular suspension.

5. The method according to claim 1, wherein the immune cell-type panel further comprises a detectable binding member specific for an immune activation marker and/or an adhesion-related marker.

6. The method according to claim 1, wherein the method further comprises dissociating the biopsy, or a sample therefrom, to prepare the cellular suspension.

7. The method according to claim 6, wherein the dissociating comprises non-enzymatic tissue homogenization.

8. The method according to claim 1, wherein the cellular suspension is further contacted with a fixation reagent.

9. The method according to claim 1, wherein the biopsy is a solid tissue biopsy.

10. The method according to claim 1, wherein the tumor is a lung cancer tumor.

11. The method according to claim 10, wherein the lung cancer tumor is a non-small cell lung cancer (NSCLC) tumor.

12. The method according to claim 1, wherein the cellular suspension is further contacted with a DNA staining reagent.

13. A kit comprising:
an immune checkpoint panel comprising a plurality of detectable immune checkpoint marker binding members; and
an immune cell-type panel comprising a plurality of detectable immune cell-type marker binding members.

14. A method of screening a subject for metastatic cancer, the method comprising:
detecting the presence of the TIL population according to the method of claim 1; and
testing the subject for a metastatic cancer based on the detected presence of the TIL population.

15. A method of treating a subject for a neoplasia, the method comprising:
detecting the presence or absence of the TIL population according to the method of claim 1; and
treating the subject for the neoplasia, wherein the treating comprises:
a systemic treatment for the neoplasia when the TIL population is detected to be present; or
a local treatment for the neoplasia when the TIL is detected to be absent.
